(19) United States
(12) Patent Application Publication
(10) Pub. No.: US 2009/0053261 A1
(43) Pub. Date: Feb. 26, 2009

(54) Modified tobacco mosaic virus particles as scaffolds for display of protein antigens for vaccine applications

(76) Inventors: John A. Lindbo, Wooster, OH (US); Kenneth E. Palmer, Owensboro, KY (US); Mark L. Smith, Davis, CA (US)

Correspondence Address: WYATT, TARRANT & COMBS, LLP, 1715 AARON BRENNER DRIVE, SUITE 800, MEMPHIS, TN, 38120-4367, US

(21) Appl. No.: 11/518,549

(22) Filed: Sep. 8, 2006

Related U.S. Application Data
(60) Provisional application No. 60/715,703, filed on Sep. 8, 2005.

(51) Int. Cl. A61K 39/00; C12N 7/00; A61P 31/00

(52) U.S. Cl. 424/199.1; 435/235.1

(57) ABSTRACT

Display of peptides or proteins in an ordered, repetitive array, such as on the surface of a virus-like particle, is known to induce an enhanced immune response relative to vaccination with the “free” protein antigen. The 2100 coat proteins comprising the rod-shaped capsid of Tobacco mosaic virus (TMV) can accommodate short peptide insertions into the primary sequence, but the display of larger protein moieties on the virion surface by genetic fusions to the capsid protein has not been possible. Since TMV lacks surface exposed residues compatible with commonly available linker chemistries, we employed a randomized library approach to introduce a reactive lysine at the externally located at the amino-terminus of the coat protein. We found that we could easily control the extent of virion conjugation and demonstrated stoichiometric biotinylation of the introduced lysine. To characterize this modular platform for the display of heterologous proteins, we bound a model antigen (streptavidin (SA)-green fluorescent protein (GFP), expressed and purified from plants) to the surface of TMV, creating a GFP-SA decorated virus particle. Rapid and quantitative determination of the level of TMV capsid decoration was accomplished by subjecting the complex to amino acid analysis and solving the family of linear equations relating the pmoles of each residue to the known amino acid composition of the complex components. We obtained a GFP-SA tetramer loading of 26%, which corresponds to display of approximately 2200 GFP moieties per intact virion. We evaluated the immunogenicity of GFP decorated virions in both mice and guinea pigs, and found augmented humoral IgG titers in both species, relative to unbound GFP-SA tetramer. In mice, we observed a detectable humoral immune response after only a single immunization with the TMV-protein complex. By demonstrating the presentation of whole proteins, this study expands the utility of TMV as a vaccine scaffold beyond that which is possible by genetic manipulation.

### CROSS-REFERENCE TO RELATED APPLICATIONS

This application is a continuation-in-Part of 60/715,703 filed Sep. 8, 2005

### FIELD OF THE INVENTION

The present invention relates to the field of genetically engineered peptide production in plants, more specifically, the invention relates to the use of tobamovirus vectors to express fusion proteins.

### BACKGROUND OF THE INVENTION

Peptides are a diverse class of molecules having a variety of important chemical and biological properties. Some examples include; hormones, cytokines, immunoregulators, peptide-based enzyme inhibitors, vaccine antigens, adhesions, receptor binding domains, enzyme inhibitors and the like. The cost of chemical synthesis limits the potential applications of synthetic peptides for many useful purposes such as large scale therapeutic drug or vaccine synthesis. There is a need for inexpensive and rapid synthesis of milligram and larger quantities of naturally occurring polypeptides. Towards this goal many animal and bacterial viruses have been successfully used as peptide carriers.

The safe and inexpensive culture of plants provides an improved alternative host for the cost-effective production of such peptides. During the last decade, considerable progress has been made in expressing foreign genes in plants. Foreign proteins are now routinely produced in many plant species for modification of the plant or for production of proteins for use after extraction. Animal proteins have been effectively produced in plants (reviewed in Krebbers et al., 1992).

Vectors for the genetic manipulation of plants have been derived from several naturally occurring plant viruses, including TMV (tobacco mosaic virus). TMV is the type member of the tobamovirus group. TMV has straight tubular virions of approximately 300.times.18 nm with a 4 nm-diameter hollow canal, consisting of approximately 2000 units of a single capsid protein wound helically around a single RNA molecule. Virion particles are 95% protein and 5% RNA by weight. The genome of TMV is composed of a single-stranded RNA of 6395 nucleotides containing five large ORFs. Expression of each gene is regulated independently. The virion RNA serves as the messenger RNA (mRNA) for the 5′ genes, encoding the 126 kDa replicase subunit and the overlapping 183 kDa replicase subunit that is produced by read through of an amber stop codon approximately 5% of the time. Expression of the internal genes is controlled by different promoters on the minus-sense RNA that direct synthesis of 3′-coterminal subgenomic mRNAs which are produced during replication (FIG. 1). A detailed description of tobamovirus gene expression and life cycle can be found, among other places, in Dawson and Lehto, Advances in Virus Research 38:307-342 (1991). It is of interest to provide new and improved vectors for the genetic manipulation of plants.

For production of specific proteins, transient expression of foreign genes in plants using virus-based vectors has several advantages. Products of plant viruses are among the highest produced proteins in plants. Often a viral gene product is the major protein produced in plant cells during virus replication. Many viruses are able to quickly move from an initial infection site to almost all cells of the plant. Because of these reasons, plant viruses have been developed into efficient transient expression vectors for foreign genes in plants. Viruses of multicellular plants are relatively small, probably due to the size limitation in the pathways that allow viruses to move to adjacent cells in the systemic infection of entire plants. Most plant viruses have single-stranded RNA genomes of less than 10 kb. Genetically altered plant viruses provide one efficient means of transfecting plants with genes coding for peptide carrier fusions. Recombinant plant viruses that express fusion proteins are formed by fusions between a viral coat protein and protein of interest. By infecting plant cells with the recombinant plant viruses of the invention, relatively large quantities of the protein of interest may be produced in the form of a fusion protein. The fusion protein encoded by the recombinant plant virus may have any of a variety of forms. The protein of interest may be fused to the amino terminus of the viral coat protein or the protein of interest may be fused to the carboxyl terminus of the viral coat protein. The protein of interest may be fused internally to a coat protein. The viral coat fusion protein may have one or more properties of the protein of interest. The recombinant coat fusion protein may be used as an antigen for antibody development or to induce a protective immune response.

Virus-like particles (VLPs) represented the first example of a recombinantly-expressed vaccine, with the licensure in the mid 1980's of two hepatitis B vaccines based on yeast-derived hepatitis B surface antigen particles (HBsAg) (Hilleman, 1992). Presently, subunit vaccines based on VLPs derived from the L1 capsid proteins of human papillomavirus (HPV) are in late stage clinical trials and have demonstrated remarkable efficacy against their targeted HPV subtypes (Harper et al., 2004; VIIIa et al., 2005). The success of these antigens in inducing protective immunity is due in large part to their highly ordered and repetitive structure. The quasicrystalline nature of VLPs facilitates pattern recognition by the specific and innate immune systems, with sustained antibody production resulting from efficient activation of B cells through surface Ig cross-linking (Bachmann et al., 1993; Bachmann, Zinkernagel, and Oxenius, 1998). These immunostimulatory properties of VLPs prompted their application as a platform for the display of defined linear epitopes from diverse pathogens. TMV, poliovirus virions, hepatitis B surface and core antigen particles, cowpea and alfalfa mosaic viruses represent a subset of the particulate virus-derived epitope carriers capable of stimulating neutralizing antibodies and in certain cases inducing protective immunity (Burke et al., 1988; Clarke et al., 1987; Dalsgaard et al., 1997; Delpeyroux et al., 1986; Delpeyroux et al., 1988; Haynes et al., 1986; Koo et al., 1999; Valenzuela, Coit, and Kuo, 1985; Yusibov et al., 1997). However, these studies did not investigate the duration of the protective immunity induced against the target pathogen. This point has been addressed in the case of the most advanced prophylactic vaccine that employs VLP epitope display; a recombinant chimeric HBsAg particle displaying a large fragment of the circumsporozoite protein from the malaria parasite Plasmodium falciparum. Several trials in humans have demonstrated that this vaccine induced both strong antibody and T-cell responses, with protection against multiple parasite genotypes. In spite of this, the protective immunity was relatively short-lived, indicating that further optimization in vaccine composition is required, to elicit sufficient memory T-helper (Th) cells to ensure an anamnestic response (Bojang et al., 2001; Stoute et al., 1998). Other studies suggest that vaccines based on display of only one or a few HLA-restricted T cell epitopes will limit the vaccine applicability to only a subset of the patient population (Birkett et al., 2002; Nardin et al., 2000). Increasing the number of pathogen specific CD4⁺-T-cell epitopes in order to remove limitation to certain HLA genotypes and to bolster induction of specific memory T-cells is therefore required. This necessarily increases the size of the protein fragment that is to be displayed, which increases the potential for introducing protein conformations that disrupt the capsid proteins primary sequence, preventing particle formation (Karpenko et al., 2000). Improved structural understanding of capsid formation (Kratz, Bottcher, and Nassal, 1999) and genetic engineering approaches (Cruz et al., 1996) have permitted the display of GFP as a genetic fusion to core antigen particles of hepatitis B and potato virus X (PVX) respectively. A single chain functional antibody (scFv) has also been displayed in the case of PVX (Smolenska et al., 1998). However, GFP and scFvs are structurally compact and the ability to extend this approach to proteins with more extended tertiary structures is unclear. Recently the ectodomain of the outer surface protein A from Borrelia burgdorferi was fused to the HBcAg capsid (Nassal et al., 2005). This fusion was significantly more difficult to generate and the final VLP preparation was heterogeneous in nature, also containing incomplete particles and polymorphic multimeric structures. In addition, these approaches may compromise the ability to recover purified VLP, particularly in the case of filamentous capsids such as PVX.

An alternative approach is to uncouple expression and purification of the capsid scaffold and the heterologous protein to be displayed, with subsequent conjugation of the desired antigen to the VLP surface. Using this strategy, biotinylated papillomavirus L1 VLPs were decorated with a streptavidin fusion of the self-polypeptide TNF- [symbol] This composition elicited high-titer protective autoantibodies in a mouse model for type II collagen-induced arthritis (Chackerian, Lowy, and Schiller, 2001). Strategies based on bifunctional cross-linkers with cowpea mosaic virus (Chatterji et al., 2004) and HBcAg (Jegerlehner et al., 2002) as scaffolds have also been described. Here, we report the implementation of a platform for the display of whole foreign proteins on the surface of TMV, in which the protein for conjugation was also transiently expressed inplanta via recombinant TMV vectors, and subsequently purified. To obtain a soluble scaffold for biotinylation that accumulated to high levels required the evolution of a recombinant TMV with a surface exposed lysine.

### SUMMARY OF THE INVENTION

In the present invention the binding properties of biotin and avidin to each other are exploited to enable the display of peptides of increased size on the surface of the virus coat protein. Another aspect of the invention is to provide polynucleotides encoding the genomes of the subject recombinant plant viruses. Another aspect of the invention is to provide the coat fusion proteins encoded by the subject recombinant plant viruses. Yet another embodiment of the invention is to provide plant cells that have been infected by the recombinant plant viruses of the invention.

We characterized conjugation of a GFP-SA fusion to this biotinylated lysine TMV capsid. Since our primary application of the TMV scaffold was to improve vaccine immunogenicity, we compared the immune response to this VLP complex to that obtained with the free streptavidin GFP fusion protein.

### UTILITY STATEMENT

This invention has utility as a a major component of a vaccine composition, and in making in making vaccines that are capable of inducing enhanced immune response relative to vaccination with the “free” protein antigen. Display of peptides or proteins in an ordered, repetitive array, such as on the surface of a virus-like particle, is known to induce an enhanced immune response relative to vaccination with the “free” protein antigen. The 2100 coat proteins comprising the rod-shaped capsid of Tobacco mosaic virus (TMV) can accommodate short peptide insertions into the primary sequence, but the display of larger protein moieties on the virion surface by genetic fusions to the capsid protein has not been possible. Since TMV lacks surface exposed residues compatible with commonly available linker chemistries, we employed a randomized library approach to introduce a reactive lysine at the externally located at the amino-terminus of the coat protein. We found that we could easily control the extent of virion conjugation and demonstrated stoichiometric biotinylation of the introduced lysine. To characterize this modular platform for the display of heterologous proteins, we bound a model antigen (streptavidin (SA)-green fluorescent protein (GFP), expressed and purified from plants) to the surface of TMV, creating a GFP-SA decorated virus particle. Rapid and quantitative determination of the level of TMV capsid decoration was accomplished by subjecting the complex to amino acid analysis and solving the family of linear equations relating the pmoles of each residue to the known amino acid composition of the complex components. We obtained a GFP-SA tetramer loading of 26%, which corresponds to display of approximately 2200 GFP moieties per intact virion. We evaluated the immunogenicity of GFP decorated virions in both mice and guinea pigs, and found augmented humoral IgG titers in both species, relative to unbound GFP-SA tetramer. In mice, we observed a detectable humoral immune response after only a single immunization with the TMV-protein complex. By demonstrating the presentation of whole proteins, this study expands the utility of TMV as a vaccine scaffold beyond that which is possible by genetic manipulation.

Presented here is a virus or virus-like particle displaying a foreign peptide sequence as a genetic fusion of to the capsid coat protein together with a mitigating peptide sequence or sequences, also present as a genetic fusion or fusions to the coat protein, such that the mitigating peptide sequence(s) improve one or more of the following characteristics of the virus or virus-like particle; a) accumulation in the host employed for production; b) yield obtained with purification; c) solubility; d) conformation of the foreign peptide sequence; and accessibility of the foreign peptide sequence.

Also presented here is a virus or virus-like particle displaying a foreign peptide sequence of from 1 to approximately 50 amino acids as a genetic fusion of to the capsid coat protein together with a mitigating peptide sequence or sequences, also present as a genetic fusion or fusions to the coat protein, such that the mitigating peptide sequence(s) improve one or more of the following characteristics of the virus or virus-like particle; a) accumulation in the host employed for production; b) yield obtained with purification; c) solubility; d) conformation of the foreign peptide sequence; and accessibility of the foreign peptide sequence.

Presented here is a virus or virus-like particle displaying a foreign peptide sequence as a genetic fusion of to the capsid coat protein together with a mitigating peptide sequence or sequences, also present as a genetic fusion or fusions to the coat protein, such that the mitigating peptide sequence(s) improve one or more of the following characteristics of the virus or virus-like particle; a) accumulation in the host employed for production; b) yield obtained with purification; c) solubility; d) conformation of the foreign peptide sequence; and accessibility of the foreign peptide sequence. Each mitigating peptide sequence consists of 1 to approximately 10 amino acids.

Also presented here is a virus or virus-like particle displaying a foreign peptide sequence as a genetic fusion of to the capsid coat protein together with a mitigating peptide sequence or sequences, also present as a genetic fusion or fusions to the coat protein, such that the mitigating peptide sequence(s) improve one or more of the following characteristics of the virus or virus-like particle; a) accumulation in the host employed for production; b) yield obtained with purification; c) solubility; d) conformation of the foreign peptide sequence; and accessibility of the foreign peptide sequence. The mitigating sequence(s) can be located at one or more of the following locations relative to the foreign peptide sequence; a) directly upstream; b) immediately downstream; c) or separated from the foreign sequence based on location within the coat protein sequence; d) or some combination of the above

Also presented here is a virus or virus-like particle displaying a foreign peptide sequence as a genetic fusion of to the capsid coat protein together with a mitigating peptide sequence or sequences, also present as a genetic fusion or fusions to the coat protein, such that the mitigating peptide sequence(s) improve one or more of the following characteristics of the virus or virus-like particle; a) accumulation in the host employed for production; b) yield obtained with purification; c) solubility; d) conformation of the foreign peptide sequence; and accessibility of the foreign peptide sequence. The foreign peptide sequence is located at or near the N-terminus of the coat protein sequence and the mitigating sequence(s) are located at or near the C-terminus of the coat protein sequence and/or in a surface exposed region of the coat protein amino acid sequence

Also presented here is a virus or virus-like particle displaying a foreign peptide sequence as a genetic fusion of to the capsid coat protein together with a mitigating peptide sequence or sequences, also present as a genetic fusion or fusions to the coat protein, such that the mitigating peptide sequence(s) improve one or more of the following characteristics of the virus or virus-like particle; a) accumulation in the host employed for production; b) yield obtained with purification; c) solubility; d) conformation of the foreign peptide sequence; and accessibility of the foreign peptide sequence. The foreign peptide sequence is located at or near the C-terminus of the coat protein sequence and the mitigating sequence(s) are located at or near the N-terminus of the coat protein sequence and/or in a surface exposed region of the coat protein amino acid sequence.

Also presented here is a virus or virus-like particle displaying a foreign peptide sequence as a genetic fusion of to the capsid coat protein together with a mitigating peptide sequence or sequences, also present as a genetic fusion or fusions to the coat protein, such that the mitigating peptide sequence(s) improve one or more of the following characteristics of the virus or virus-like particle; a) accumulation in the host employed for production; b) yield obtained with purification; c) solubility; d) conformation of the foreign peptide sequence; and accessibility of the foreign peptide sequence. The foreign peptide sequence is located within a surface exposed region of the coat protein amino acid sequence and the mitigating sequence(s) is located at or near the C-terminus of the coat protein sequence and/or at or near the N-terminus of the coat protein sequence and/or within a surface exposed region of the coat protein amino acid sequence other than the one occupied by the foreign peptide sequence.

Also presented here is a virus or virus-like particle displaying a foreign peptide sequence as a genetic fusion of to the capsid coat protein together with a mitigating peptide sequence or sequences, also present as a genetic fusion or fusions to the coat protein, such that the mitigating peptide sequence(s) improve one or more of the following characteristics of the virus or virus-like particle; a) accumulation in the host employed for production; b) yield obtained with purification; c) solubility; d) conformation of the foreign peptide sequence; and accessibility of the foreign peptide sequence. The virus or virus-like particle displaying a foreign peptide sequence is derived from a population of virus or virus-like particles where the mitigating sequence or sequences consist of a randomly generated library of amino acids and selection was based on one of the properties listed in claim 6.

Also presented here is a virus or virus-like particle displaying a foreign peptide sequence as a genetic fusion of to the capsid coat protein together with a mitigating peptide sequence or sequences, also present as a genetic fusion or fusions to the coat protein, such that the mitigating peptide sequence(s) improve one or more of the following characteristics of the virus or virus-like particle; a) accumulation in the host employed for production; b) yield obtained with purification; c) solubility; d) conformation of the foreign peptide sequence; and accessibility of the foreign peptide sequence. The foreign peptide sequence consists of a single amino acid, either lysine or cysteine and the randomly generated mitigating sequence is three amino acids in length.

Also presented here is a virus or virus-like particle displaying a foreign peptide sequence as a genetic fusion of to the capsid coat protein together with a mitigating peptide sequence or sequences, also present as a genetic fusion or fusions to the coat protein, such that the mitigating peptide sequence(s) improve one or more of the following characteristics of the virus or virus-like particle; a) accumulation in the host employed for production; b) yield obtained with purification; c) solubility; d) conformation of the foreign peptide sequence; and accessibility of the foreign peptide sequence. The virus is the tobacco mosaic virus.

Also presented here is a biotinylated virus or virus-like particle, where a virus or virus-like particle displaying a foreign peptide sequence as a genetic fusion of to the capsid coat protein together with a mitigating peptide sequence or sequences, also present as a genetic fusion or fusions to the coat protein, such that the mitigating peptide sequence(s) improve one or more of the following characteristics of the virus or virus-like particle; a) accumulation in the host employed for production; b) yield obtained with purification; c) solubility; d) conformation of the foreign peptide sequence; and accessibility of the foreign peptide sequence, and the virus or virus-like particle is combined with a biotin analog capable of conjugating to the lysine or cysteine of the foreign peptide sequence, such that the biotin is covalently attached to the virus coat protein. The virus can be a tobacco mosaic virus.

Also presented here is a biotinylated virus or virus-like particle, where a virus or virus-like particle displaying a foreign peptide sequence as a genetic fusion of to the capsid coat protein together with a mitigating peptide sequence or sequences, also present as a genetic fusion or fusions to the coat protein, such that the mitigating peptide sequence(s) improve one or more of the following characteristics of the virus or virus-like particle; a) accumulation in the host employed for production; b) yield obtained with purification; c) solubility; d) conformation of the foreign peptide sequence; and accessibility of the foreign peptide sequence, and the virus or virus-like particle is combined with a NHS-PEO₄-biotin capable of conjugating to the lysine or cysteine of the foreign peptide sequence, such that the biotin is covalently attached to the virus coat protein. The virus can be a tobacco mosaic virus.

Another embodiment is a display scaffold, comprising a virus particle comprising coat proteins displaying streptavidin by genetic fusion. The scaffold is capable of displaying a variety of different biotinylated peptides. The display scaffold can be an assembled virus or virus-like particle, or a partially assembled virus or virus-like particle, or one or more viral coat protein fusion protein with streptavidin, capable of binding to biotin.

Another embodiment is a biotinylated display scaffold, comprising an assembled virus particle comprising coat proteins displaying streptavidin by genetic fusion. The scaffold may be biotinylated by either in vivo or in vitro methods.

Another embodiment is a biotinylated display scaffold, comprising an assembled virus particle comprising coat proteins displaying streptavidin by genetic fusion. The streptavidin is biotinylated by either in vivo or in vitro methods so that a biotinylated peptide is bound to the streptavidin. The biotinylated peptide can have any function.

In another embodiment, the biotinylated peptide is an antigen capable of eliciting an immune reaction in an animal

Another embodiment is a composition comprising a biotinylated peptide that is an antigen capable of eliciting an immune reaction in an animal

Another embodiment is a vaccine composition comprising a peptide that is an antigen capable of eliciting an immune reaction in an animal, the peptide being biotin bound and attached to the display scaffold of the present invention by streptavidin-biotin binding.

Another embodiment is a vaccine composition comprising a peptide that is an antigen capable of eliciting an immune reaction in an animal, the peptide being biotin bound and attached to the display scaffold of the present invention by streptavidin-biotin binding, and at least a liquid solvent, or liquid capable of making a suspension, slurry or solvent mixture.

In another embodiment, the vaccine is an oral vaccine.

### BRIEF DESCRIPTION OF FIGURES

FIG. 1 is a Vector map of pLSB 1289.

FIG. 2 GFP-AviTag protein sequence. The AviTag sequence (a substrate for E. coli Biotin protein ligase) is bold and underlined.

FIG. 3. pLSB 1293 Vector Map

FIG. 4 pLSB 1290 Vector Map.

FIG. 5 Amino acid sequence of GFP-SA fusion protein.

Streptavidin core sequence is in bold.

FIG. 6. Nucleotide sequence of GFP-SA fusion gene.

Streptavidin core coding sequence is in bold.

FIG. 7 ELISA results of pooled sera samples, bleeds 2 and 3.

FIG. 8 Dilution end point analysis (log 10 based dilution scale) of bleed 3 sera samples of individual mice in each treatment group. Sera was diluted in 1×PBS, 2% BSA to 1:10, 1:100, 1:1000, 1:10,000 and 1:50,000 for each mouse. Diluted samples were used as primary antibody for GFP coated plates, secondary antibody

FIG. 9 pLSB 1295 Vector Map.

FIG. 10 Nucleotide sequence of CP fusion library in vector 1295.

Inserted sequences are in bold and underlined.

FIG. 11 pLSB 1296 Vector Map

FIG. 12 (inserted) nucleotides are in bold.

FIG. 13 pLSB 2900 Vector map.

FIG. 14 Nucleotide sequence of CP fusion in vector 2900.

Inserted nucleotide sequences are in boldface type.

FIG. 15 pLSB 2901 Vector Map

FIG. 16 Nucleotide sequence of CP fusion in vector 2901.

Inserted nucleotides are in boldface.

FIG. 17 pLSB 2902 Vector Map.

FIG. 18 Nucleotide sequence of CP fusion in vector 2902.

Inserted sequence is in bold typeface.

FIG. 19 pLSB 2903 Vector Map.

FIG. 20 Nucleotide sequence of CP fusion in vector 2903.

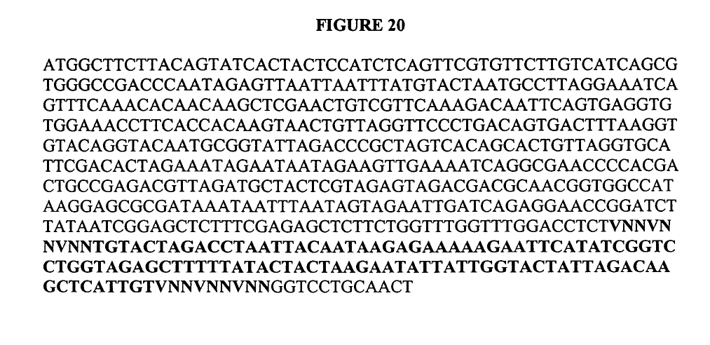

Inserted sequence is in bold typeface.

FIG. 21 pLSB 2904 Vector Map.

FIG. 22 Nucleotide sequence of CP fusion in vector 2904.

Inserted (non-TMV CP sequence) is in boldface.

FIG. 23. pLSB 2905 Vector Map.

FIG. 24 Nucleotide sequence of CP fusion in vector 2905.

Non-TMV sequence is in bold.

FIG. 25 pLSB 2907 Vector Map.

FIG. 26 Nucleotide sequence of CP fusion in vector 2907.

Non-TMV sequences are in bold.

FIG. 27 pLSB 2908 Vector Map.

FIG. 28 Nucleotide sequence of CP fusion in vector 2908.

Inserted nucleotide sequences are in bold.

FIG. 29. Validation of COPV PsV Assembly and Transduction of HEK 293 Cells. Various dilutions of COPV PsV stock were added to HEK 293 cells as described (Buck et al., 2004; Pastrana et al., 2004), and secreted alkaline phosphatase activity in supernatant media assayed after three day incubation. A 1:800 dilution of COPV PsV induced an appropriate amount of SEAP expression for use in a virus neutralization assay.

FIG. 30. Neutralization of COPV PsV by COPV mAb 7-2, but not mAb 14-1. The percentage reduction in SEAP activity in wells of cells incubated with COPV mAb is indicated. Assays were repeated in triplicate, and the error bars reflect the between-well variation for triplicate assays.

FIG. 31. The amino acid sequence of COPV L2 Protein (Genbank accession # NP_{—}056818). Underlined sequence indicates the region selected to produce the recombinant L2:SA fusion.

FIG. 32. A. Process flow diagram for the principal steps in COPV L2-SA purification, from infected plant tissue through affinity chromatography. B. SDS-PAGE analysis for initial COPV L2-SA purification. Legend; GJ, homogenized plant extract “Green Juice”; S1, initial supernatant; 25P, 25% saturated ammonium sulfate pellet; 25S, 25% saturated ammonium sulfate supernatant; L, load (dialyzed 50% ammonium sulfate fraction); FT, flow through; M12, Invitrogen Mark 12 protein marker; 50P=50% ammonium sulfate pellet; DP, precipitate from 50% ammonium sulfate fraction dialysis; DS=supernatant from 50% ammonium sulfate fraction dialysis; F## eluant fractions; M, monomer form of COPV L2-SA; R, rubisco large and small subunits; TMV, TMV coat protein.

FIG. 33. A. Optimized process flow diagram for the principal steps in COPV L2-SA purification, from infected plant tissue through affinity chromatography. B. SDS-PAGE analysis for the optimized COPV L2-SA purification process. Legend; GJ, homogenized plant extract “Green Juice”; S1, initial supernatant; 25P, 25% saturated ammonium sulfate pellet; 25S, 25% saturated ammonium sulfate supernatant; L, load (25% ammonium sulfate fraction); FT, flow through; M12, Invitrogen Mark 12 protein marker; F## eluant fractions; M, monomer form of COPV L2-SA; TMV, TMV coat protein.

FIG. 34. Molecular weight mass spectrometry for affinity purified COPV L2-SA, prior to BEI treatment. Full length COPV L2-SA (aa 1-242) has an expected molecular weight of 25025.97 Da (M+H). With methionine cleaved (aa 2-242) the expected molecular weight is 24894.77 Da (M+H), which matches the major peak observed (24896.75 Da) within the 0.05% confidence interval. No acetylation of the N-terminal glycine was detected.

FIG. 35. Band shift analysis for the COPV L2 streptavidin fusion, alone and complexed to biotinylated 1295.4 TMV capsids. A. Schematic diagram of a streptavidin (SA) antigen (Ag) fusion and its quaternary structure as a function of temperature and the presence of biotin. B. SDS-PAGE migration pattern for the COPV L2-SA fusion (L2 SA) alone or mixed with unbiotinylated 1295.4 TMV (TMV). No samples were BEI treated and when biotin was added it was present at a 5-fold molar excess. C. SDS-PAGE migration pattern for 1295.4 TMV, COPV L2 SA and the COPV L2 SA, biotinylated TMV mixture (L2-SA/Bt TMV). All samples were BEI treated and when biotin was added it was present at a 5-fold molar excess. Legend; T, tetramer; M, monomer; CP, TMV coat protein; Cmplx, complex of COPV L2 SA tetramer and biotinylated coat protein; Bt CP, biotinylated coat protein.

FIG. 36 COPV C1 7-2 Light Chain

FIG. 37 COPV C1 7-2 Heavy Chain

FIG. 38 COPV C1 7-2 FAB

FIG. 39 COPV 1 7-2 mAb

FIG. 40 HPV-16 G4 FAB

FIG. 41 HPV-16 G4 mAb

FIG. 42 COPV L2-SA

The nucleotide and deduced amino acid sequence of the recombinant L2:SA fusion protein (242 a.a.; 25 kDa). The amino acid sequence derived from COPV L2 domain is shown in bold typeface. Underlined nucleotides indicate the NgomIV and AvrII cloning sites.

### DETAILED DESCRIPTION OF THE INVENTION

Tobacco mosaic virus has proven an effective carrier for the display of foreign peptides, successfully eliciting a neutralizing immune response against numerous target pathogens and in one reported case, breaking B-cell tolerance and inducing auto-reactive antibodies (reviewed in Smith et al. (in press) and Pogue et al. (2002)). However, the display of peptide fragments exceeding 25-30 amino acids has been problematic. In addition, the amino acid composition of the epitope influences its compatibility with genetic fusion. To expand the utility of TMV as an antigen carrier and overcome the antigen size limitations associated with genetic modification, we investigated the use of a biotin-decorated capsid for the presentation of antigen streptavidin fusions. Biotin was introduced by using NHS-PEO₄-biotin, an amine reactive conjugate of the vitamin. Although the TMV coat protein contains two lysine residues (amino acids 54 and 69), predicted to delineate the surface exposed 60's loop, their reactivity with the biotin conjugate was low, suggesting limited solvent exposure. Since the terminal amino group was also unreactive, owing to its acetylation in planta, the introduction of a reactive surface exposed amine was required. Initially, a lysine spanned by two glycine residues was fused to the N-terminus of CP, yielding the recombinant virus LSB 2800. When purified this fusion had a propensity to aggregate, even at low protein concentrations. The mechanism by which the addition of a basic residue results in such poor solubility characteristics is not clear. A library based approach was employed to obtain a soluble TMV capsid with a solvent exposed lysine, by introducing three random amino acids upstream of an N-terminal lysine. From the small number of isolates sequenced (Table 1) a clear bias was evident for the introduction of acidic residues, suggesting the need to mitigate the presence of the added positive charge. Charge separation was also preferred, with one or two predominantly aromatic or aliphatic residues separating the acidic residue and the lysine for the majority of the isolates, pLSB 1295.6 being the exception. Purified virus was obtained for 8 of the 11 isolates and no correlation was observed between the frequency of a particular mitigating sequence and the rTMV yield, although the virus titers obtained represented a notable improvement over LSB 2800. Furthermore, the majority of the LSB 1295 virus series remained soluble when stored at greater than 10 mg/ml, as illustrated for LSB 1295.4 the rTMV carried forward in this study. Recently, Demir and Stowell (2002) generated a lysine displaying virion by introducing a single amino acid mutation at the C-terminus of the native coat protein (T158K). To permit comparison with the mitigating sequence capsids of the 1295 library, we cloned, expressed and purified the T158K recombinant. Purification was performed as outlined by Demir and Stowell, as well as by the modified Gooding and Herbert (1967) procedure that we employed for both LSB 2800 and the 1295 virion series (see Materials and Methods), and identity was confirmed by mass spectrometry (data not shown). The T158K mutant accumulation was 25-30 fold higher than for LSB 2800, with levels in the infected tissue reaching 3 mg/g fresh weight and depending on the purification route, yields of purified virus ranged from 0.9 to 1.5 mg/g fresh weight. To assess lysine accessibility, the T158K and LSB 1295.4 virus preparations were combined with NHS-PEO₄-biotin, with the biotin conjugate present at either a 24 or 240-fold molar excess. Virus concentration was normalized to 0.8 mg/ml and incubation was for 24 hours and room temperature. By protein gel electrophoresis, we observed a similar level of biotin conjugation to both the T158K and LSB 1295.4 virions, suggesting the degree of solvent exposure for the lysines in the two coat protein contexts was comparable. However, in our hands the T158K virus showed a marked propensity to aggregate. When the T158K virus was centrifuged briefly at 15,000×g for 5 minutes, less than 10% remained in the supernatant, irrespective of the purification route employed. In contrast, when LSB 1295.4 virus was centrifuged in parallel, >98% remained soluble, highlighting the benefits of the mitigating sequence library approach. Excessive aggregation of the TMV scaffold will probably hinder processing of the assembled complexes whether they be employed in vaccinology, other medicinal applications or as self-assembled templates for the formation of nanomolecular wires for use in nanoscale electronic devices as was proposed by others (Demir and Stowell, 2002).

There are conflicting reports regarding the tolerance of TMV for the addition of positively charged amino acids. The display of two peptides tested by Bendahmane et al (1999), containing one or four basic residues, resulted in the formation of necrotic lesions on the inoculated leaves, a phenomenon that could be counteracted by the introduction of acidic residues. However, positively charged peptides fused to TMV showing no deleterious host phenotypes and which were easily purified have also been documented (Wu et al., 2003). Our experience with positively charged epitopes spans this spectrum, with the purified virus fusions also showing a range of solubilities that can, in addition, be influenced by whether the peptide is inserted at the N or C terminus (LSBC, unpublished results). The breadth of results suggests that generalizations are of limited benefit and that library based selection strategies may constitute a relatively rapid approach to overcome the negative effects of any given epitope fused to TMV CP, or other viral capsid proteins. We have employed this library-based approach for lysine display at the C-terminus of TMV (inserted prior to GPAT), as well as for the introduction of a surface exposed cysteine residue at either terminus of the U1 coat protein. In all cases the solubility and accumulation of these evolved recombinant virions was superior to the addition of either GKG or GCG and the virions were compatible with multiple homo and heterodimer linker chemistries (LSBC, unpublished results). Even greater latitude may be provided by separating the epitope and mitigating sequence, by for example placing them at the N and C terminus respectively, or by including multiple stretches of mitigating sequence. These approaches may also improve TMV CP compatibility with protein fragments exceeding 30 amino acids.

The extent of capsid biotinylation was readily controlled through adjustment of the molar ratio of coat protein to biotin conjugate, reaction temperature and time. To obtain stoichiometric conjugation of the LSB 1295.4 virus, employed at a concentration of 0.8 mg/ml, a 240-fold molar excess of the NHS-PEO₄-bitoin was required, with room temperature incubation for 48 hours. The efficiency with which the surface exposed amine reacts with the NHS ester, via nucleophilic attack, is strongly dependent on protein concentration, with hydrolysis of the NHS ester, the major competing reaction, occurring more readily in dilution protein solutions. By increasing the 1295.4 virion concentration 5 fold, the CP:biotin conjugate ratio required to achieve 100% loading was reduced by a factor of 10 (data not shown). Modification of the native coat protein was necessitated by the fact that the amino acid side chains commonly targeted for modification are not represented on the virion surface. Recently new linker modalities have been designed permitting the chemical modification of the native U1 coat protein and offering an alternative strategy to the use of a recombinant TMV scaffold for antigen display (Schlick et al., 2005). Biotin peptide mimics offer yet another means for antigen display, eliminating the need for chemical modification entirely. The C-terminus of TMV has been successfully modified to display the streptavidin-specific heptapeptide sequence TLIAHPQ and tetramer association with this recombinant virion was demonstrated (Negrouk et al., 2004). However, similar to 2-iminobiotin (Hofmann et al., 1980), streptavidin affinity for this peptide mimic is orders of magnitude lower than for biotin, with disassociation occurring under acidic conditions. These characteristics will likely alter the immunological characteristics of the complex and may be attractive in certain cases, for example, rapid antigen unloading following uptake into the acidic lysosomal compartment may facilitate antigen cross-presentation, which is crucial in antiviral immunity (Lizee et al., 2003). The breadth of choices in the available capsid scaffolds introduces the possibility of tailoring the vaccines characteristics to the generation of the most appropriate immune response for the target disease. However, the utility of these approaches will only be determined empirically.

Typically streptavidin fusions are expressed in bacterial systems, and functional protein is recovered from the isolated inclusion bodies following solubilization and refolding (Kipriyanov et al., 1995; Ohno and Meruelo, 1996; Sano and Cantor, 1991) with typical yields of a few milligrams per 100 ml of culture (Sano and Cantor, 2000). In the case of antigens whose neutralizing epitopes are conformational in nature, there is always the concern that the native tertiary structure may not be recovered. The successful production of soluble and functional recombinant streptavidin in E. Coli has been achieved (Gallizia et al., 1998), with yields of 70 mg per liter of culture, however, this approach does not yet appear to have been extended to streptavidin fusions. For mammalian pathogen antigens and self-antigens, eukaryotic-expression systems may offer improved epitope authenticity and circumvent the need for protein refolding. In the case of plants, there have only been a limited number of reports describing the expression of streptavidin and avidin, and the production of fusions thereof does not appear to have been described. Constitutive avidin expression in tomato was detrimental to plant development (Ginzberg et al., 2004) owing to reduced biotin availability, while expression in maize resulted in partial or total male sterility (Hood et al., 1997), although accumulation at levels of 2-3% of extractable seed protein were reported. Another study in tobacco demonstrated that toxicity could be circumvented by vacuolar targeting, with streptavidin levels reaching 1.5% of total soluble protein (Murray et al., 2002). By employing a transient TMV-vector expression system, plant development and foreign protein expression are temporally segregated, facilitating the expression of streptavidin fusion proteins in the absence of toxic side effects. For the GFP streptavidin fusion, this permitted the recovery of 60-120 mg of functional GFP-SA/kg tissue, which compares favorably with bacterial expression and obviates the need for a refolding step. We have obtained similar yields of purified protein for other fusion partners to streptavidin, indicating that TMv-vectors constitute a good alternative expression system for these chimeric proteins (LSBC, unpublished data). Further improvements may be possible by coupling TMV-vector expression with targeted accumulation, such as to the vacuole, the chloroplast or the endoplasmic reticulum. Targeting to the latter compartment may also be beneficial for antigens requiring post-translational modifications or the chaperone components of the eukaryotic ER and golgi systems for correct folding. In addition, we employed the native S. avidinii sequence, which is biased towards a high GC content. This could potentially introduce undesirable secondary structure into the TMV expression vector, thereby reducing the translation efficiency of the subgenomic RNAs. A plant codon-optimized sequence for streptavidin is currently being synthesizing and will be compared to constructs employing the native DNA sequence.

Amino acid analysis of the two-component (GFP-SA/LSB 1295.4 coat protein) complex permitted the quantitative determination of its composition. The use of amino acid analysis for composition determination can be extended to any protein complex provided the individual components to be combined are sufficiently characterized, i.e. by mass spectrometry. Up to 21 linear algebraic equations can be derived relating the pmoles of each protein to the pmoles of a given amino acid, as outlined in equation 1. When only two components are present, this introduces a high degree of redundancy, permitting the independent determination of the moles of each protein using pairs of equations and averaging of the results. This procedure could in theory be extended to complexes consisting of up to 21 unique components, although consideration of amino acids sensitive to the protein hydrolysis procedure will reduce the number of valid equations. For example cysteine, methionine and tryptophan are destroyed during the 6N HCl hydrolysis. For Cys and Met prior oxidation with performic acid is possible, yielding the acid stable forms cysteic acid and methionine sulfone. Also for the stable amino acids, caution must be exercised in the equations chosen. As indicated in the Results, the complex components were initially subjected to amino acid analysis individually, to ensure selection of only the amino acids that were accurately and reproducibly quantified. For example, with the chromatography and peak integration parameters employed, the isoleucine composition was consistently underestimated by 16-18%. Therefore the equation based on this amino acid was not employed as the error would be propagated, yielding incorrect and/or potentially meaningless solutions, e.g. negative pmoles of a given protein component. This procedure may also be employed to determine the degree of loading for peptides that are chemically conjugated to the capsid scaffold, although the accuracy with which the pmoles of each amino acid can be determined will be of even greater importance in this case. In certain cases the peptides may be appropriately tagged to facilitate quantitation of conjugation efficiency; the TMV coat protein lacks histidine, therefore for this capsid a polyhisitine tagged peptide would be appropriate, as this would provide a unique signature in the chromatogram trace.

The size distribution of the biotinylated TMV capsids was altered following decoration with the streptavidin fusions, with the predominant rod length decreasing from 300 mm to 50-100 nm. The assembly of neutravidin onto the surface of biotinylated TMV, already coated onto copper grids, has previously been reported (Demir and Stowell, 2002). A similar level of decoration was observed, however, no comment was made regarding alterations to the particle size distribution. The GFP-SA decorated virions were subject to a series of PEG precipitations, to eliminate unbound streptavidin fusion prior to analysis. It is possible that fragmentation occurred with this additional processing, although no change in the particle size distribution was observed for control samples (unbiotinylated TMV mixed with GFP-SA) processed in parallel. An alternative explanation is that the packing of the GFP-SA tetramers onto the capsid surface exceeded a critical limit, resulting in rupture of the TMV rod. The SDS detergent, added at 2% w/v with no heating, disrupts TMV virion yielding free coat protein (data not shown), whereas biotinylated coat protein-streptavidin association can occur in the presence of SDS and is stable up to 60° C. This disparity in stability suggests that the affinity of the GFP-SA for the biotinylated coat protein may be sufficient for rod disassociation when the GFP-SA is present in molar excess, as was the case here. Since the extent of capsid surface biotinylation can be easily controlled, this possibility can be addressed experimentally and if necessary a level of biotinylation chosen that will give complete surface coverage, while maintaining intact 300 nm rods. The influence of the complex dimensions on the immune response may also merit investigation. Early studies in rabbits administered either ¹⁴C-labelled TMV virions or disassociated coat protein demonstrated that the particulate nature of TMV resulted in improved uptake by and activation of antigen presenting cells (APCs), as the intact capsids were more effectively and rapidly transported from the site of injection to proximal lymph nodes, and then to the spleen (Loor, 1967). Further studies with streptavidin fusion loaded rods, of different size distributions, would permit the influence of rod length on the potency of the immune response to be evaluated.

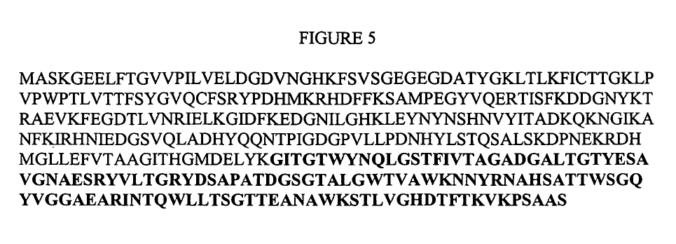

In the current study, we used GFP as a model antigen. Clearly, physical association of the streptavidin fusion tetramers to TMV capsids augmented the immune response induced by the TMV complexes, relative to free tetramer alone or tetramer mixed with unbiotinylated rods (FIG. 5). After the second immunization, total IgG titers were improved 4 to 20-fold in both mice and guinea pigs, and with mice antibodies were detectable after the first immunization for animals administered the GFP-SA TMV complex. Given that robust induction of specific T-cell responses is desirable for many vaccines, we have also compared the induction of vaccine-specific CD8⁺ T cells by the GFP-SA/rTMV complex, to the response obtained with uncomplexed antigen. We immunized BALB/c mice with either GFP-SA or GFP-SA/rTMV, at a normalized dose of 1 □g or 10 □g GFP. One week after the second immunization, spleens were harvested and isolated lymphocytes stimulated with 10⁻⁶ M of the H-2d restricted peptide HYLSTQSAL from GFP (Gambotto et al., 2000). At the higher GFP dose, rTMV association resulted in a significant increase in activated CD8⁺ T cells. For the 1 □g GFP dose, 2 out of 4 animals in the complex group showed a detectable response, while no induction observed for the free antigen (manuscript in preparation). These encouraging results with the GFP model system have provided a rationale for ongoing studies in our laboratory, which focus on the application of this TMV platform to a series of antigen targets from human papillomavirus and human immunodeficiency virus. Facile systems for the display of heterologous proteins on the surface of icosahedral capsids and their therapeutic potential have been reported (Chackerian, Lowy, and Schiller, 2001; Jegerlehner et al., 2002). The current work extends capsid display for therapeutic applications to another geometry, that of rod shaped capsids, thereby increasing the density with which whole antigens may be displayed compared to competing VLP systems. On a T=3 particle such as hepatitis B core antigen VLP, 180 tetramers can be theoretically accommodated, while the capacity of a T=7 particle such as papillomavirus is 420. The 26% loading obtained in the present study corresponds to 550 tetraners per intact virion, and for cases where the antigen fusion partner to streptavidin is smaller than GFP (30 kDa), a further increase in the TMV packing density is possible, while for the icosahedral platforms it will remain constant.

### EXAMPLE 1

### Production of Biotinylated GFP in E. coli

Biotinylation is a post translational modification of some proteins, carried out by a biotin protein ligase enzyme. Typically less than 6 different protein species are biotinylated in any one cell type, making this a relatively rare post translational modification. Biotin protein ligase attaches a biotin to the epsilon amino group of specific lysine (K) residues.

The E. coli BirA gene encodes for a biotin protein ligase enzyme. By screening a peptide library for the ability to act as a substrate for the E. coli birA enzyme, researchers have identified a 16 aa peptide which can be biotinylated by birA. This 16 aa tag is referred to as the “aviTag” sequence (GLNDIFEAQKIEWHEG). Proteins with this tag, at either the N or C terminus, can be biotinylated by birA.

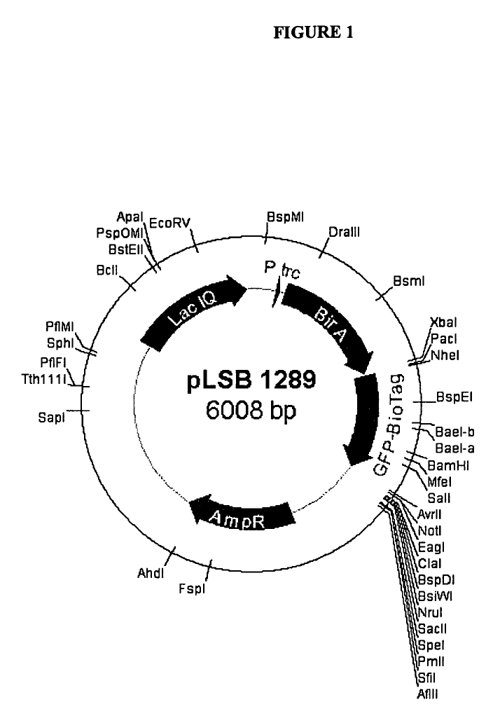

The bacterial expression vector pSE380 (invitrogen) was modified to express both the E. coli Bir A (biotin protein ligase enzyme) and a GFP-aviTag fusion protein to generate the vector pLSB 1289. FIG. 1 pLSB 1289 Vector map.

PCR amplification of E. coli BirA Gene:

E. coli genomic DNA was purified from DH5a E. coli using Qiagen DNeasy kit according to manufacturers instructions. Five nanograms of purified genomic E. coli DNA was used in a 50 ul PCR reaction with oligos JAL 604 Forward oligo seq (TTGTTAATTAACCATGGGAAAGGATAACACCGTGCCACTGAAATTG) and JAL 605 Reverse oligo sequence: (CTTTCTAGATTATTTTTCTGCACTACGCAGGGATATTTCA) and Pfu Turbo DNA polymerase, for 30 cycles of 94 C 30 seconds, 54 C 1 min, 72 C 1 min. The approximately 1 kb PCR product was digested with NcoI and XbaI and cloned into PacI-XbaI digested pSE 380 to generate pSE380:BirA.

### Construction of GFP-AviTag Fusion:

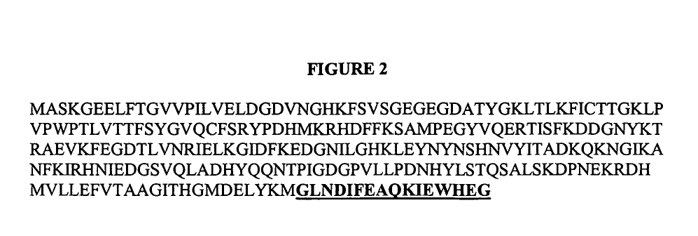

PCR amplify up GFP gene with oligos JAL 606 forward oligo (GGGTCTAGAGAAGGATTAATTAAATGGCTAGCAAAGGAGAAG) which has an XbaI site for cloning, the E. coli ribosome binding site, and an ATG codon for the GFP gene, and JAL 607 Reverse oligo (GTGCCTCGAATATATCATTTAAACCAGATTTGTAGAGCTCATCCATGCCA) which includes a portion of the AviTag coding sequence. The remaining portion of the avitag coding sequence, and an AvrII site, for cloning purposes, was generated on the 606-607 pcr product by re-amplifying the 606-607 PCR product with JAL 606 and JAL 608 reverse primer (CCCCCTAGGTTAACCCTCATGCCACTCTATTTTTTGTGCCTCGAATATATCATTTAAA C) LSBC Notebook 1306, JAL Notebook 25). This PCR product was digested with XbaI-AvrII and ligated into XbaI-AvrII digested pSE 380:BirA. The resulting plasmid was named pLSB 1289. The amino acid sequence of the GFP-avitag fusion protein is presented in FIG. 1. FIG. 2 is a GFP-AviTag protein sequence. The AviTag sequence (a substrate for E. coli Biotin protein ligase) is bold and underlined.

Production and Purification of Biotinylated GFP from E. coli Cultures.

Cultures of E. coli were grown in LB or superbroth with 1 mM IPTG overnight. Cells were pelleted by centrifugation. Pelleted cells were lysed with BugBuster reagent (Novagen) using lysozyme and nuclease (Epicenter) to reduce viscosity of the lysate. (Notebook 1334) Solid ammonium sulfate was added to 25% saturation. Preciptating proteins were discarded, supernatant saved and ammonium sulfate added to 50% saturation. Precipitated proteins were resuspended in pH 7.5 phosphate buffer (either 50 or 100 mM). Biotinylated GFP was affinity purified on Softlink Resin (Promega catalog #V201) according to manufacturers instructions.

### EXAMPLE 2

### Production of Biotinylated TMV U5 CP in Plants

### Procedures:

PCR Amplification of E. coli BirA Gene:

E. coli genomic DNA was purified from DH5a E. coli using Qiagen DNeasy kit according to manufacturers instructions. Five nanograms of purified genomic E. coli DNA was used in a 50 ul PCR reaction with oligos JAL 604 Forward oligo seq (TTGTTAATTAACCATGGGAAAGGATAACACCGTGCCACTGAAATTG) and JAL 605 Reverse oligo sequence: (CTTTCTAGATTATTTTTCTGCACTACGCAGGGATATTTCA) and Pfu Turbo DNA polymerase, for 30 cycles of 94 C 30 seconds, 54 C1 min, 72 C1 min. The approximately 1 kb PCR product was digested with PacI and XbaI and cloned into PacI-AvrII digested p30B GFP derivative.

### Construction of a U5CP-AviTag Fusion.

Initial attempts to fuse the avitag peptide sequence to the N-terminus of the U5 CP resulted in a fusion protein that did not accumulate well in plants. Therefore the avitag was also fused to the C-terminus of the U5 CP.

Using PCR based insertional mutagenesis the coding sequence for the avitag of the amino acid sequence (GLNDIFEAQKIEWHEG) was inserted upstream of the final 5 codons of the U5 CP. The U5 CP-AviTAG gene fusion was inserted into a TMV based vector which contained the E. coli Bir A gene to produce the plasmid pLSB 1293. T7 transcripts of pLSB 1293 were inoculated onto N. benthamiana plants. Virus was purified from infected tissue using a pH 7.2 buffer, as the CP-avitag fusion virus was not soluble at pH 5.0.

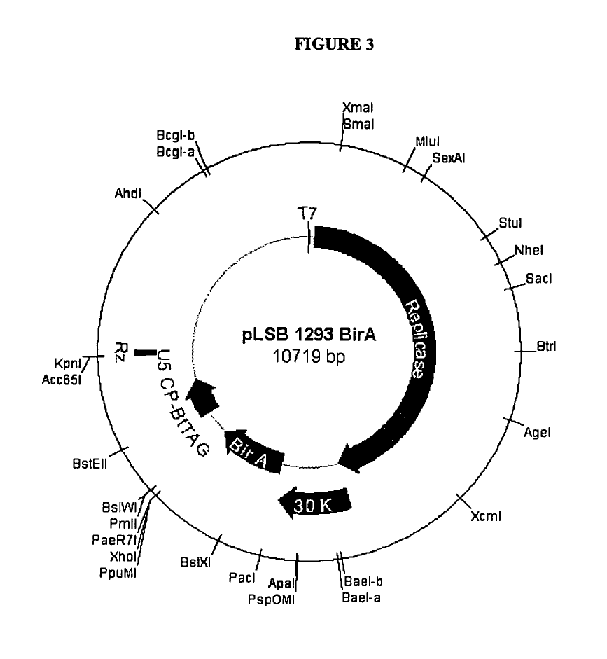

To analyze CP for the presence of biotin a “Western” blot was performed in which protein bound to nitrocellulose was probed with Streptavidin-alkaline phosphatase fusion protein. FIG. 3. is a vector map of pLSB 1293.

### Summary:

Although biotinylated U5 CP was detected, the amount of biotinylated protein was probably quite low. I did not try to quantitate it, but it seemed very low (I guess less than 1% of subunity biotinylated). Since the in vitro biotinylation of virus with lysine residues on the near N-terminus was more controllable, I pursued the in vitro biotinylation methods.

### EXAMPLE 3

### Production of Biotinylated Proteins In Vitro

### Quantitative Biotinylation of TMV Particles In Vitro.

T7, capped transcripts from pLSB 1295.4 DNA sample were used to inoculate N. benthamiana plants. Virus was purified from infected tissue using pH 5.0 acetate buffer, 50 C heat treatment followed by PEG/NaCl precipitation according to standard purification conditions.

Non-native U1-CP amino acids are in bold.

MADFKSYSITTPSQFVFLSSAWADPIELINLCTNALGNQFQTQQARTVVQ
RQFSEVWKPSPQVTVRFPDSDFKVYRYNAVLDPLVTALLGAFDTRNRIIE
VENQANPTTAETLDATRRVDDATVAIRSAINNLIVELIRGTGSYNRSSFE
SSSGLVWTSGPAT

The reagent NHS-PEO₄-Biotin (Pierce Cat # 21329) reacts with amine groups (e.g. lysine residues) and can be used to conjugate biotin to proteins. Quantitative biotinylation of TMV 1295.4 (see Table 1) could be obtained by preparing 400 ug of purified 1295.4 virus in 350 ul of 50 mM phosphate buffer (pH 7.0), and using this solution to resuspend 0.2 mg of “No-Weigh” NHS-PEO4-Biotin. Biotinylation reaction was allowed to proceed for at least 4 hours at room temperature, and then analyzed on a 16% SDS PAGE gel, followed by staining with coomassie blue. A slight size shift between biotinylated virus and non-biotinylated 1295.4 virus control sample could be detected by this analysis. (Notebook JAL 32).

Biotinylated virus was purified and concentrated by precipitation in 4% PEG and 0.6M NaCl according to standard methods.

### EXAMPLES 4

### Purification of Streptavidin Fusion Proteins from Plants

Affinity Purification of Geneware Produced GFP-SA Fusion Protein from Plants.

### Production of a GFP-Streptavidin Fusion Protein Gene.

Purified Streptomyces avidinii genomic DNA was obtained from ATCC. The core streptavidin coding sequence (ca. 500 bp) was PCR amplified from genomic DNA with oligos JAL 609 Forward oligo (ATGAGCTCTACAAAGGTATCACCGGCACCTGGTACAACCAGCTC) and JAL 610 Reverse primer (AAACCTAGGTTAGGAGGCGGCGGACGGCTTCACCTTGG) (Notebook 25 pg100). Five nanograms purified DNA was used in a 100 ul PCR reaction, using Pfu Turbo DNA polymerase (Stratagene) according to manufacturers suggestions. Oligo JAL 609 generates a SacI site in the PCR product, and JAL 610 generates an AvrII site in the PCR product.

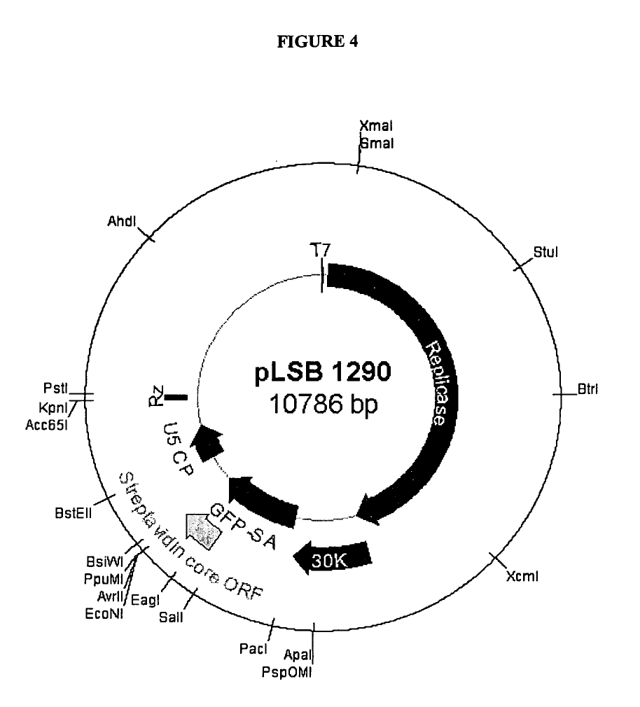

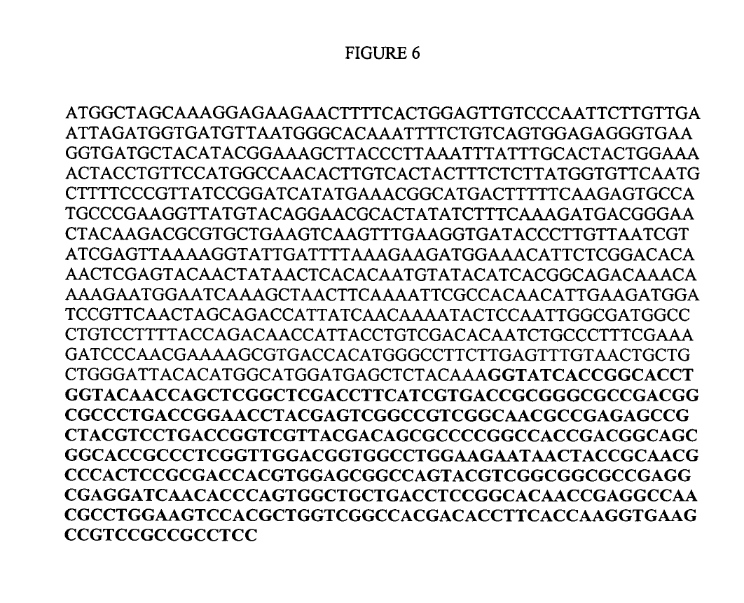

A p30B GFPc3 derivative (a TMV-based dual subgenomic vector containing the GFPc3 gene) was further modified to express a GFP-Streptavidin (SA) fusion by ligating the SA gene PCR product into the SacI site near the 3′ end of GFP using standard cloning methods. The resulting plasmid pLSB 1290 was transcribed with Ambion T7 mMessage mMachine and transcripts used to inoculate N. benthamiana plants. FIG. 4 is a vector map of pLSB 1290 FIG. 5 is an Amino acid sequence of GFP-SA fusion protein. Streptavidin core sequence is in bold. FIG. 6. is a Nucleotide sequence of GFP-SA fusion gene.

Streptavidin core coding sequence is in bold.

Purification of GFP-SA Fusion Protein from Plants.

Infected plant tissue was ground in 3 volumes extraction buffer (100 mM phosphate pH 7.2, 0.01% Na-metabisulfite, 1 ul BME per ml) in a blender. Ground tissue was filtered through cheesecloth, heated to 52 to 55 C for 7 minutes then centrifuged at 12K×g for 10 minutes to clarify. Solid ammonium sulfate was added to 25% saturation and held on ice for at least 2 hours (to precipitate virus). Sample was centrifuged at 12K×g for 10 minutes, and supernatant transferred to clean tube. Ammonium sulfate was added to 50% saturation and sample held at 4 C overnight.

GFP-SA was pelleted from 50% saturated ammonium sulfate solution by centrifugation for 10 minutes at 12K×g. Pellet was resuspended in 1/30th original extract volume and dialyzed overnight into 50 mM phosphate pH 7.2, 50 mM NaCl. Add 0.04 mls 10% acetic acid per ml of dialyzed extract. Incubate at 42 C 5 minutes, centrifuge 12k×g for 12 min. Save supernatant and dialyze into 25 mM phosphate pH 7.2

Partially purified GFP-SA was finally affinity purified using immobilized iminobiotin resin (Pierce Cat # 20221) according to manufacturers instructions. Briefly, 1.5 mls iminobiotin beads (prewashed in pH 11, 50 mM Ammonium carbonate, 500 mM NaCl) were added to 7 mls of partially purified GFP-SA extract. The sample was mixed on an inverting shaker at 4 C for about 1 hour. After batch binding, beads were packed into a column and beads washed according to manufacturers instructions. GFP-SA fusion was eluted from beads with 0.1M Acetic acid. Eluted samples were neutralized with an equal volume of 1M pH 8.0 Tris, and dialized into 50 mM pH 7.2 phosphate, 100 mM NaCl overnight. Final protein concentration was estimated by a BCA assay, and protein purity estimated by SDS-PAGE gel analysis of samples. (JAL notebook 32)

### EXAMPLE 5

### Binding Streptavidin Fusion Proteins to Biotinylated Virion Particles Applications

This technology can be used to display streptavidin fusion proteins on the surface of TMV particles. This approach can be used in vaccine studies. Streptavidin fusion proteins can be generated in a variety of systems and affinity purified using immobilized imminobiotin. Biotinylated TMV particles can be generated in vitro. When combined, the streptavidin fusions bind to the biotinylated virus particles.

Association of GFP-SA with Biotinylated TMV Particles.

Biotinylated TMV particles were combined with purified GFP-SA protein and incubated at 4 C for overnight to several days. Virus (and GFP-SA associated with it) was precipitated out of the sample by adding ammonium sulfate to 25% saturation, or by adding PEG to 4% and NaCl to 0.6 M, following by centrifugation for 10 min at 12K×g. Final virus pellet was resuspended in 1×PBS in preparation for animal inoculations.

This could also be done as follows: To 100 ug biotinylated virus, add 400 ug of GFP SA, at a final [ ] of 5 mg/ml GFP-SA (i.e. 80 ul) if solution is not this concentrated, place in speed vac an reduce volume until it is about 80 ul in volume. Let sample sit at least overnight at 4 C. Mix occasionally by pipetting. Precipitate virus with PEG and NaCl. Resuspend in 500 ul×PBS. Place in eppendorf tube shaker to resuspend virus/GFP-SA pellet. This is preferred over pipetting to mix, as the pellet tends to be kind of stick initially and can stick to pipette tip. Use 20 ul in BCA assay to estimate protein.

### Analysis of GFP-SA and Biotinylated TMV Complex.

Precipitated TMV-Bt+GFP-SA was analyzed by BCA assay and SDS-PAGE. Two volumes 5×SDS PAGE loading dye were added to a volume of sample and boiled for 20 minutes. The sample was then analyzed on 16% SDS PAGE along with a standard curve of known quantities of TMV CP, to estimate the amount of virus CP in the sample.

### Proteins Bound to TMV Particles are Immunogenic

### Animal Inoculations:

Mice (BALB/C, female, 4-6 wks) received 3 SC inoculations 14 days apart. Tail bleed samples were taken 7-8 days post each inoculation. Efforts were made to inoculate animals with equimolar amounts of GFP antigen. See Table 2 for immunization details.

TABLE 1
Mouse immunization with Native GFP or GFP-SA fusions.

| | (6/15) | 6/29) | (7/13) |
|---|---|---|---|
| | Inoc 1 | Inoc 2 | Inoc 3 |
| PBS | 100 ul | 100 ul | 100 ul |
| GFP | 8 ug | 10 ug | 9 ug |
| GFP-SA | 11.6 ug | 15 ug | 12.5 ug |
| GFP-SA + | 11.6 ug GFPSA + | 15 ug + | 12.5 ug + |
| TMV 1295.4 | 10 ug TMV | 20 ug TMV | 2.5 ug TMV |
| GFP-SA + | 11.6 ugGFPSA + | 15 ug + | 12.5 ug + |
| TMV-Bt | 10 ug TMV-Bt | 20 ug TMV-Bt | 2.5 ug TMV-Bt |

### ELISA Analysis of Sera.

ELISA plates were coated with purified native GFP. Mouse sera was diluted in 1×PBS, 2% BSA and used as primary antibody. Rabbit anti-mouse HRP conjugate was used as secondary antibody. Plate was developed with One-Step turbo TMB ELISA reagent.

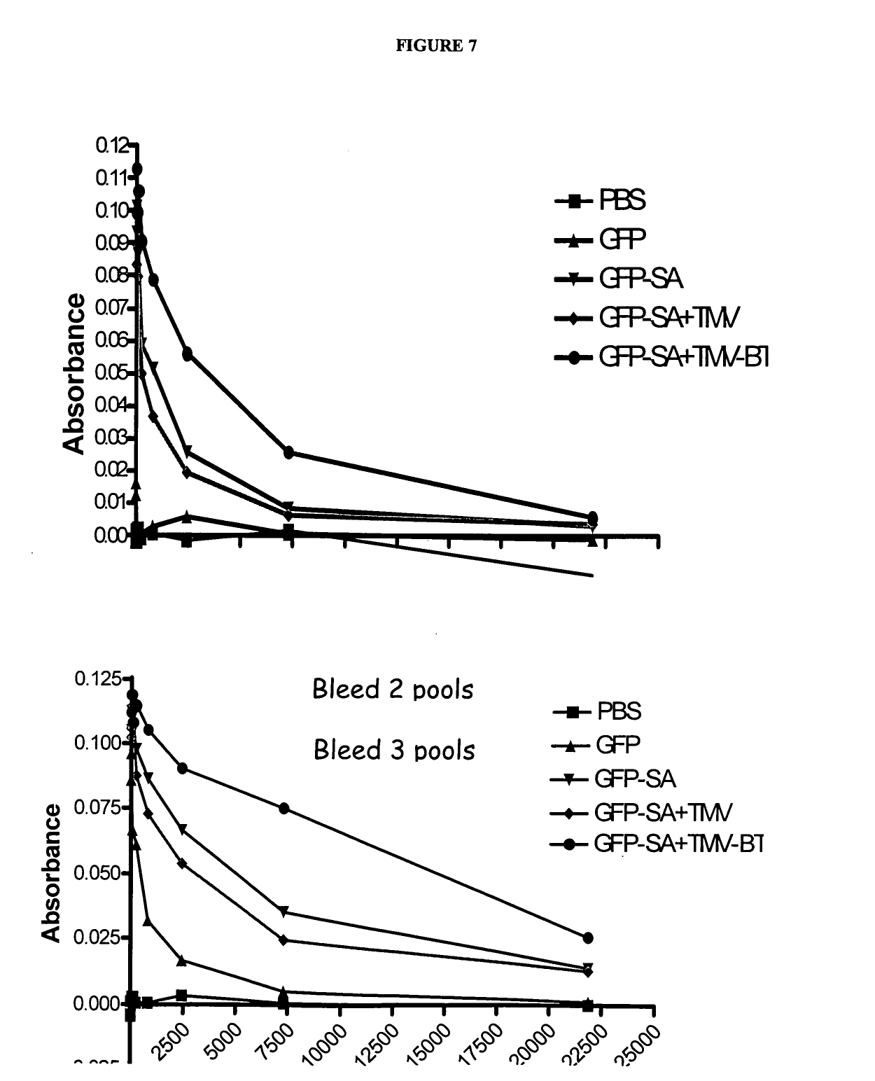

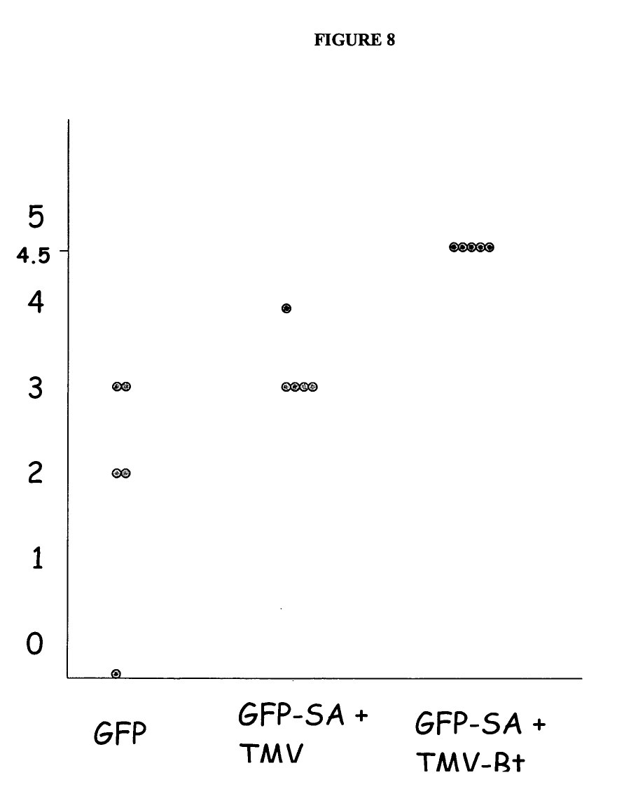

Equal volumes of sera samples from all 5 mice in each treatment group were combined and serially diluted in 1×PBS, 2% BSA. Various dilutions of pooled sera samples from bleeds 2 and 3 were used as the primary antibody on GFP coated plates, and incubated at room temp 1 hour. Plates were washed with 1×TTBS, then 1:6000 dilution of Rabbit anti mouse HRP (diluted in 1×PBS in 2% BSA) added as secondary antibody. Plates were incubated 1 hour at room temp, washed with 1×TTBS and plate developed with one-step turbo TMB ELISA reagent (FIG. 5). FIG. 7 shows ELISA results of pooled sera samples, bleeds 2 and 3. FIG. 8 shows Dilution end point analysis (log 10 based dilution scale) of bleed 3 sera samples of individual mice in each treatment group. Sera was diluted in 1×PBS, 2% BSA to 1:10, 1:100, 1:1000, 1:10,000 and 1:50,000 for each mouse. Diluted samples were used as primary antibody for GFP coated plates, secondary antibody was 1:6000 dilution of Rabbit anti mouse HRP (diluted in 1×PBS in 2% BSA). Plates were developed with one-step turbo TMB ELISA reagent (Pierce). Positive values were at least 2 times the value of negative control (sear from PBS inoculated animals).

### EXAMPLE 6

### Construction of pLSB 1298 Vector for Cloning CP Libraries

### CP PacI-BsiWI Deletion Mutant

Purpose: To ensure that no wild-type CP background vectors appear in various CP libraries a PacI-BsiWI deletion mutant (of most of the CP coding sequence) was Generated from a full length clone of TMV U1, which had the endogenous NcoI site (in the 30K gene) mutated, and a unique NcoI site generated at the start codon of the U1 CP gene. See map below.

Vector is to be digested with NcoI and KpnI. Cloning a functional CP and 3′ uts in as a NcoI KpnI fragment will generate a functional full length virus sequence.

### EXAMPLE 7

### Mitigating Sequence Libraries for TMV CP Fusions

### Methods for Generating, Screening and Analyzing Mitigating Sequence Libraries on the U1 CP Gene N Terminal Mitigating Library for a Lysine Residue (pLSB 1295 Library)

Insertion of a Lysine Codon onto the 5′ end of the TMV U1 CP orf.

Using PCR based mutagenesis approach, three randomized codons and a lysine codon were fused to the 5′ end of the U1 CP ORF. The TMV CP gene and 3′ UTS were amplified from a full length clone of TMV (p801) using oligos JAL 613 Forward direction oligo, (CGAACCATGGNNNNKNNKAAATCTTACAGTATCACTACTCCATCTCA) and JAL 590 Reverse direction oligo. (gcc aac aca tcc g gg tac c tg ggc ccc ta). The resulting PCR product of about 800 bp was digested with NcoI and KpnI and ligated into NcoI, KpnI digested pJL 150/254 to produce pLSB 1295 library. (pJL 150/254 is a modified form of TMV U1 cDNA in which the naturally occurring NcoI site in the TMV movement protein has been destroyed by a point mutation, and a unique NcoI site was generated at the ATG (start) codon of the TMV CP. This plasmid also has a KpnI site at the very 3′ end of the viral cDNA. (see JAL notebook 26)).

This ligation was transformed into DH5a E. coli and plated on LB-Agar plates with 100 ug/ml ampicillin.

About 3600 colonies (of pLSB 1295) were resuspended off of agar plates and pooled. DNA was purified from the E. coli, linearized with KpnI and transcribed using Ambions' T7 mMessage mMachine transcription kit, according to manufacturers instructions.

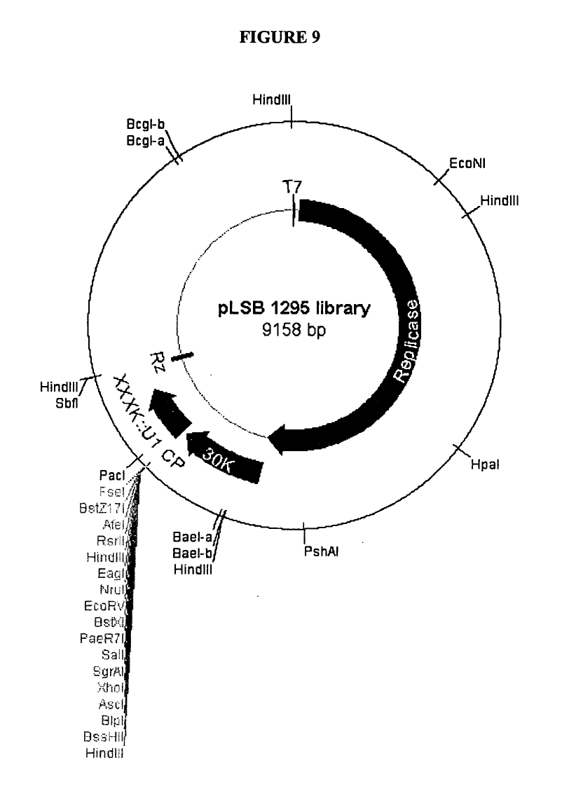

Transcripts were encapsidated in purified TMV CP (Mark Smith, personal communication). Encapsidated RNA was combined with FES and used to inoculate 12 N. benthamina plants, and onto the TMV local lesion host N. tabacum (N) leaf, to estimate the number of infectious units in the inoculum. Based upon these results each N. benth plant was inoculated with approximately 100 infectious units. FIG. 9 shows a vector map of pLSB 1295.

Approximately 8-10 days post inoculation one systemically infected leaf from each N. benth plant was harvested. Leaves from all 12 inoculated plants were pooled and virus purified from the leaves as follows: Infected leaves were ground to a powder in a mortar and pestle using liquid nitrogen. Four volumes of extraction buffer (50 mM Acetate buffer (pH 5.0), 0.01% Na-metabisulfate, 1 ul BMe per ml) was added to the ground tissue and ground until well liquified. Extract was filtered through cheesecloth/miracloth and held at 50 C for 5-10 minutes. Heat treated sample was centrifuged for 10 minutes at 10K×g. Supernatant was transferred to a clean tube. 40% PEG (mw 8000) was added to a final [ ] of 4% and SM NaCl was added to a final [ ] of 0.6 M. Sample was mixed well by inverting, and place on ice for at least 45 minutes. Virus was collected by centrifuging for 10 minutes at 12K×g. Virus pellet was resuspended in approximately 1/10th original extract volume of 50 mM Phosphate buffer, pH 7.2.

Virus was diluted 10-1, 10-2, 10-3 and 10-4 in FES buffer. Four N. benth plants were inoculated with each virus dilution (20 ul per plant). About 8 days post inoculation when all plants were systemically infected, one systemically infected leaf of each plant was collected and virus purified from the pooled leaves, as described above. This passage and purification protocol was repeated one more time.

After the final virus purification, RNA was purified from the pooled virus prep using Qiagen RNAeasy kit, according to manufacturers instructions. The CP gene was amplified from the viral RNA sample using the Promega ImPromII RT-PCR kit as follows: One microgram of purified RNA was primed with 10 pmoles JAL 619 oligo (GCCTTGGTACCTGGGCCCCTACCGGGGGTAACGG). Two microliters of RT reaction were used in a PCR reaction using oligos JAL 619 and JAL 618 (cgatgatgattcggaggctactg) which sits about 300 bp upstream of the U1 CP start codon. The resulting PCR product was digested with NcoI and KpnI the fragment containing the CP orf isolated from an agarose gel and ligated into NcoI-KpnI digested pJL 150/254.

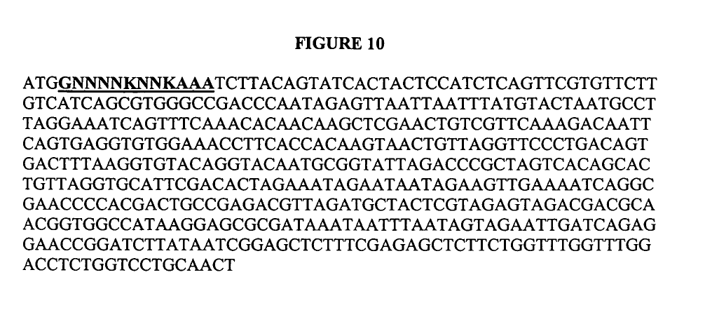

The ligation reaction was transformed into DH5a E. coli and plated on LB-Agar plates containing 100 ug/ml ampicillin. DNA was purified from cultures from individual colonies and analyzed by DNA sequence analysis using JAL 153 (aggctactgtcgccgaatc) as a sequencing primer. The deduced amino acid sequence of some of the CPs genes cloned from this experiment are presented in Table 1. Select clones were transcribed using Ambion's T7 mMessage mMachine kit, encapsidated in U1 CP, as described above, and inoculated onto at N. benthamiana plants. FIG. 10 shows the Nucleotide sequence of CP fusion library in vector 1295. Inserted sequences are in bold and underlined.

TABLE 5.1

CP amino acid sequences of select 1295
isolates.

| | |
|---|---|
| Isolate number | CP AA sequence |

| | |
|---|---|
| wt TMV U1 | MSYSITTP . . . |
| pLSB 1295.1 | MAEFKSYSITTP . . . |
| pLSB 1295.2 | MEVLKSYSITTP . . . |
| pLSB 1295.3 | MAEVKSYSITTP |
| pLSB 1295.4 | MADFKSYSITTP . . . |
| pLSB 1295.6 | MDVEKSYSITTP . . . |
| pLSB 1295.9 | MEGAKSYSITTP . . . |
| pLSB 1295.10 | MEPMKSYSITTP . . . |
| pLSB 1295.11 | MEMGKSYSITTP . . . |
| pLSB 1295.12 | MDGAKSYSITTP . . . |

Note:
Additional pLSB 1295 isolates were sequenced in June 2004. See notebook 1388, Approximately 8-10 days post inoculation virus was purified from infected N. benthamiana plants (as previously described). Samples of purified virus were analyzed by SDS PAGE and MALDI TOF to confirm the presence of additional amino acids on the CPs.

### EXAMPLE 8

### Mitigating Library for a Lysine Residue at the GPAT Position of U1 CP

### pLSB 1296 Library

Insertion of a Lysine Codon onto the 3′ end of the TMV U1 CP orf.

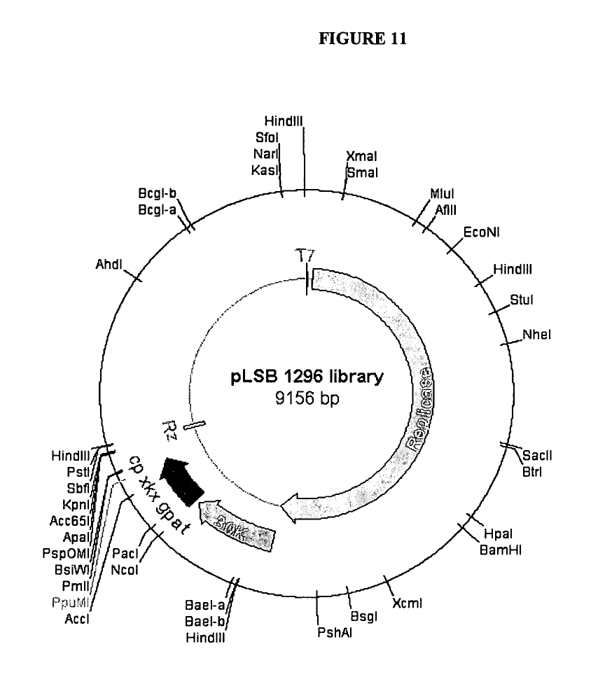

Using PCR based mutagenesis approach, a lysine codon and two flanking degenerate codons, were inserted before the final 4 codons of the CP, which encode for the amino acids GPAT. The mutagenized CP orf was cloned into a full length TMV cDNA backbone to generate the library pLSB 1296. FIG. 11 is a vector map of pLSB 1296.

The pLSB 1296 library ligation was transformed into DH5a E. coli and plated on LB-Agar plates with 100 ug/ml ampicillin. (LSBC notebook 1334, JAL notebook 27).

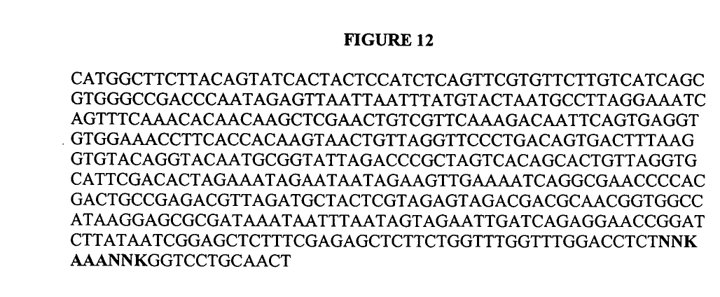

About 3600 colonies (of pLSB 1296) were resuspended off of agar plates and pooled. DNA was purified from the E. coli, linearized with KpnI and transcribed using Ambions' T7 mMessage mMachine transcription kit, according to manufacturers instructions. FIG. 12 (inserted) nucleotides are in bold.

Transcripts were encapsidated in purified TMV CP (Mark Smith, personal communication). Encapsidated RNA was combined with FES and used to inoculate 12 N. benthamina plants, and onto the TMV local lesion host N. tabacum (N) leaf, to estimate the number of infectious units in the inoculum. Based upon these results each N. benth plant was inoculated with approximately 100 infectious units.

Approximately 8-10 days post inoculation one systemically infected leaf from each N. benth plant was harvested. Leaves from all 12 inoculated plants were pooled and virus purified from the leaves as follows: Infected leaves were ground to a powder in a mortar and pestle using liquid nitrogen. Four volumes of extraction buffer (50 mM Acetate buffer (pH 5.0), 0.01% Na-metabisulfate, 1 ul BMe per ml) was added to the ground tissue and ground until well liquified. Extract was filtered through cheesecloth/miracloth and held at 50 C for 5-10 minutes. Heat treated sample was centrifuged for 10 minutes at 10K×g. Supernatant was transferred to a clean tube. 40% PEG (mw 8000) was added to a final [ ] of 4% and 5M NaCl was added to a final [ ] of 0.6 M. Sample was mixed well by inverting, and place on ice for at least 45 minutes. Virus was collected by centrifuging for 10 minutes at 12K×g. Virus pellet was resuspended in approximately 1/10th original extract volume of 50 mM Phosphate buffer, pH 7.2.

Virus was diluted 10-1, 10-2, 10-3 and 10-4 in FES buffer. Four N. benth plants were inoculated with each virus dilution (20 ul per plant). About 8 days post inoculation when all plants were systemically infected, one systemically infected leaf of each plant was collected and virus purified from the pooled leaves, as described above. This passage and purification protocol was repeated one more time. (JAL Notebook 27).

After the final virus purification, RNA was purified from the pooled virus prep using Qiagen RNAeasy kit, according to manufacturers instructions. The CP gene was amplified from the viral RNA sample using the Promega ImPromII RT-PCR kit as follows: One microgram of purified RNA was primed with 10 pmoles JAL 619 oligo (GCCTTGGTACCTGGGCCCCTACCGGGGGTAACGG). Two microliters of RT reaction were used in a PCR reaction using oligos JAL 619 and JAL 618 (cgatgatgattcggaggctactg) which sits about 300 bp upstream of the U1 CP start codon. The resulting PCR product was digested with NcoI and KpnI the fragment containing the CP orf isolated from an agarose gel and ligated into NcoI-KpnI digested pJL 150/254.

The ligation reaction was transformed into DH5a E. coli and plated on LB-Agar plates containing 100 ug/ml ampicillin. DNA was purified from cultures from individual colonies and analyzed by DNA sequence analysis using the primer “585 lA” (from Amanda Lasnik) as a sequencing primer. The deduced amino acid sequence of some of the CPs genes cloned from this experiment are presented in Table 1. Select clones were transcribed using Ambion's T7 mMessage mMachine kit, encapsidated in U1 CP, as described above, and inoculated onto at N. benthamiana plants.

TABLE 5.2

CP amino acid sequences of select 1296
isolates.

| | |
|---|---|
| Isolate | CP sequence |

| | |
|---|---|
| wt U1 CP | LVWTSGPAT |
| 1296.1 | LVWTSSNAT |
| 1296.5 | LVWTSNKEGPAT |
| 1296.7 | LVWTSAT |

Note: Of the 8 randomly selected clones sequences, 6 had the sequence LVWTSSNAT. Only one isolate pLSB 1296.5, had the desired lysine residue. It therefore appears that the amino acids GPAT are NOT required for the CP to be functional.

### EXAMPLE 9

### Mitigating Library for an N-Terminal Cysteine Residue

### pLSB 2900 Library

Using PCR based mutagenesis, the U1 CP ORF and 3′ UTS was amplified from p801 template DNA using the oligos JAL 634 Forward direction oligo seq (CGACCATGGNNNNKNNKTGTTCTTACAGTATCACTACTCCATCT) and JAL 590 Reverse oligo (gcc aac aca tcc g gg tac c tg ggc ccc ta). The oligo JAL 634 introduces 3 randomized codons before a TGT codon for cysteine, and the U1 CP sequence). The PCR product was digested with NcoI and KpnI and ligated into NcoI-KpnI digested pLSB 1298. pLSB 1298 is a partial CP gene deletion (PacI-BsiWI deletion) of pJL 150/254.

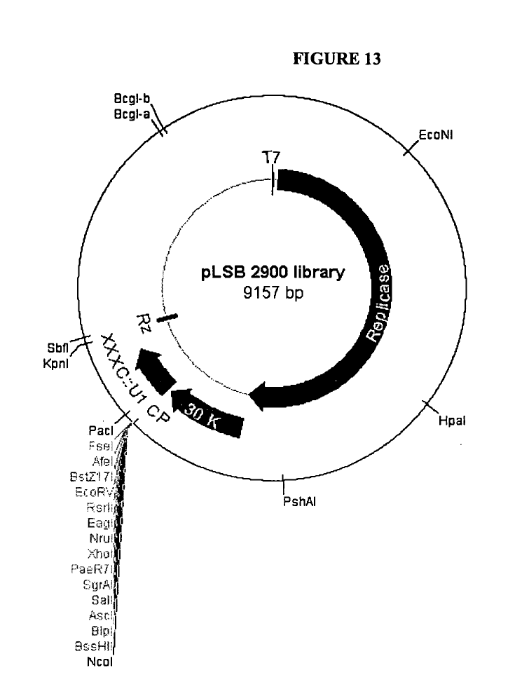

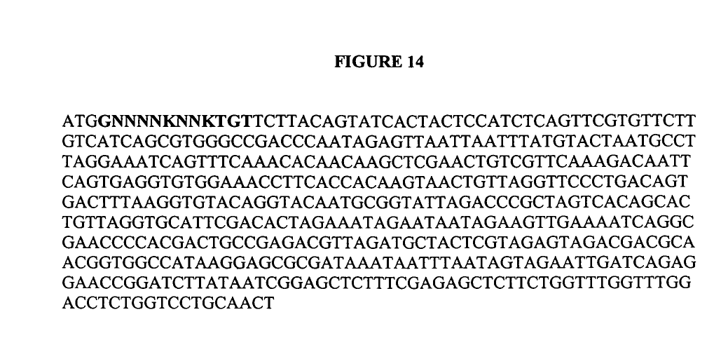

The resulting library of clones were named pLSB 2900 library members. FIG. 13 is a vector map of pLSB 2900 FIG. 14 shows the nucleotide sequence of CP fusion in vector 2900. Inserted nucleotide sequences are in boldface type.

A pool of about 18,000 clones of the 2900 library were transcribed and transcripts inoculated onto N. benthamiana and an N. tabacum N gene plant. 12 N. benth plants, each inoculated with about 125 infectious units were inoculated in this study. Plants were allowed 7-10 days to develop systemic symptoms. One systemically infected leaf from each N. benth plant was harvested. All harvested leaves were pooled and virus extracted from the pool of leaves as follows:

Infected leaves were ground to a powder in a mortar and pestle using liquid nitrogen. Four volumes of extraction buffer (50 mM Acetate buffer (pH 5.0), 0.01% Na-metabisulfate, 1 ul BMe per ml) was added to the ground tissue and ground until well liquified. Extract was filtered through cheesecloth/miracloth and held at 50 C for 5-10 minutes. Heat treated sample was centrifuged for 10 minutes at 10K×g. Supernatant was transferred to a clean tube. 40% PEG (mw 8000) was added to a final [ ] of 4% and 5M NaCl was added to a final [ ] of 0.6 M. Sample was mixed well by inverting, and place on ice for at least 45 minutes. Virus was collected by centrifuging for 10 minutes at 12K×g. Virus pellet was resuspended in approximately 1/10th original extract volume of 50 mM Phosphate buffer, pH 7.2. Sample was centrifuged for 10 minutes at 12K×g to clarify, and the supernatant saved.

### Cloning and Sequence Analysis of Recombinant Viruses.

After the final virus purification, RNA was purified from the pooled virus prep using Qiagen RNAeasy kit, according to manufacturers instructions. The CP gene was amplified from the viral RNA sample using the Promega ImPromII RT-PCR kit as follows: One microgram of purified RNA was primed with 10 pmoles JAL 619 oligo (GCCTTGGTACCTGGGCCCCTACCGGGGGTAACGG). Two microliters of RT reaction were used in a PCR reaction using oligos JAL 619 and JAL 618 (cgatgatgattcggaggctactg) which sits about 300 bp upstream of the U1 CP start codon. The resulting PCR product was digested with NcoI and KpnI the fragment containing the CP orf isolated from an agarose gel and ligated into NcoI-KpnI digested pLSB 1298.

The ligation reaction was transformed into DH5a E. coli and plated on LB-Agar plates containing 100 ug/ml ampicillin. DNA was purified from cultures from individual colonies and analyzed by DNA sequence analysis using JAL 153 (aggctactgtcgccgaatc) as a sequencing primer. The deduced amino acid sequence of some of the CPs genes cloned from this experiment are presented in Table 1. Select clones were transcribed using Ambion's T7 mMessage nMachine kit, encapsidated in U1 CP, as described above, and inoculated onto at N. benthamiana plants.

TABLE 5.3

CP amino acid sequences of select 2900
isolates.

| | |
|---|---|
| Isolate Name | CP seq |

| | |
|---|---|
| wt U1 CP | M SYSITTP . . . |
| pLSB 2900.1 | MDQGCSYSITTP . . . |
| pLSB 2900.3 | MVGACSYSYITTP . . . |
| pLSB 2900.4 | MVVGCSYSYITTP . . . |
| pLSB 2900.5 | MVEGCSYSYITTP . . . |
| pLSB 2900.7 | MVAMCSYSYITTP . . . |
| pLSB 2900.8 | MAGGCSYSYITTP . . . |
| pLSB 2900.9 | MASTCSYSYITTP . . . |
| pLSB 2900.12 | MERTCSYSYITTP . . . |

### EXAMPLE 10

### Mitigating Library for a Cysteine Residue at the GPAT Position of U1 CP

### pLSB 2901 Library

### Insertion of cys Residue at “GPAT” Position of CP

The U1 CP orf was amplified by PCR from p801 template DNA with oligos JAL 70 forward oligo (cgtccatggcttcttacagtatca) and JAL 635 Reverse direction oligo (ggaccmnnacamnnagaggtccaaaccaaaccagaagagc). This PCR product (of about 500 bp) was joined by sticky rice to the approx 200 bp PCR product of oligos JAL 614 F oligo (CCTGCAACTTGAGGTAGTCAAGATGCATAAT) and JAL 590 reverse oligo (gcc aac aca tcc g gg tac c tg ggc ccc ta) from p801 template DNA. In the sticky rice joining reaction equimolar amounts of the 2 PCR products were combined in 1×NEB ligase buffer with 0.2 mM each dATP, dTTP, and 0.25 U T4 DNA polymerase, 0.5 U T4 DNA kinase, and 300 cohesive end units NEB T4 DNA ligase. Reaction was incubated at room temperature for approximately 2 hours, then heat inactivated at 75 C for 20 min. Two microliters of the sticky rice reaction was used as template for a PCR reaction with oligos JAL 70 and JAL 590. The resulting PCR product of about 700 bp was digested with NcoI and KpI and ligated into NcoI-KpnI digested pLSB 1298 to generate library pLSB 2901.

DNA was prepared from the library similar as to described for the pLSB 2900 library. Transcription of library, inoculation onto plants, virus purification, and RT-PCR of purified viral RNA, cloning and sequencing of the pLSB 2901 library isolates was similar as to that described for pLSB 2900.

The deduced amino acid sequences of some of the CP orfs recovered from the 2901 library are in Table 2:

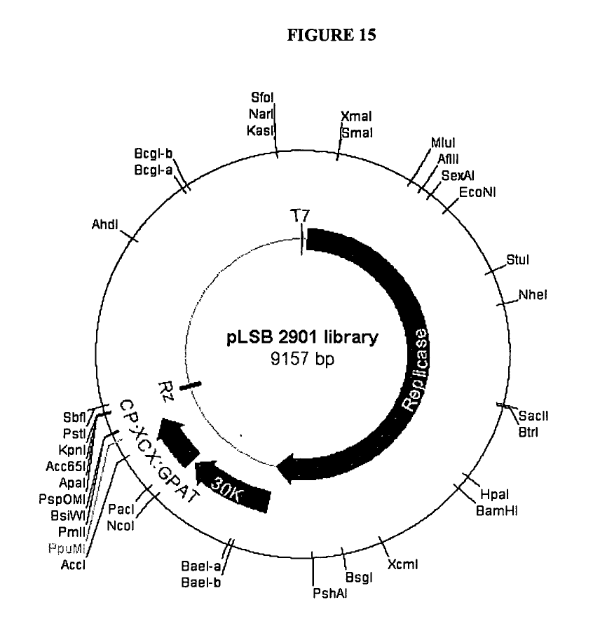

Using a PCR based mutagenesis strategy a cysteine codon, flanked by degenerate codons, were inserted into the U1 CP coding sequence, immediately upstream of the final four codons, which code for the amino acids GPAT. This mutagenized CP ORF was cloned into a full length TMV cDNA backbone to generate the library pLSB 2901. (FIG. 15 pLSB 2901 Vector Map)

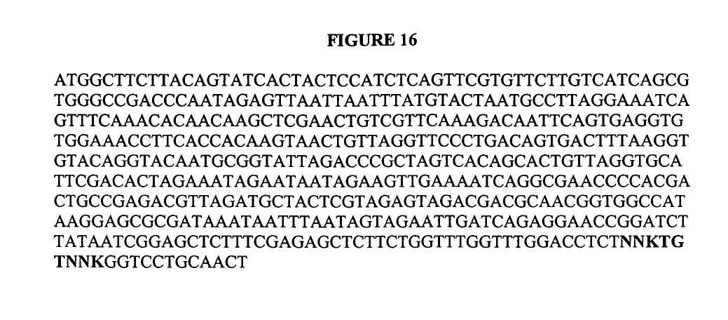

FIG. 16 shows the Nucleotide sequence of CP fusion in vector 2901. Inserted nucleotides are in boldface.

A library of about 1500 clones from the pLSB 2901 ligation were pooled and DNA purified from the pool. After transcription with Ambion's T7 mMessage mMachine, transcripts were encapsidated and inoculated onto N. benthamiana plants. (notebook 1364, JAL notebook 30)

Plants were allowed 7-10 days to develop systemic symptoms. One systemically infected leaf from each N. benth plant was harvested. All harvested leaves were pooled and virus extracted from the pool of leaves as follows:

Infected leaves were ground to a powder in a mortar and pestle using liquid nitrogen. Four volumes of extraction buffer (50 mM Acetate buffer (pH 5.0), 0.01% Na-metabisulfate, 1 ul BMe per ml) was added to the ground tissue and ground until well liquified. Extract was filtered through cheesecloth/miracloth and held at 50 C for 5-10 minutes. Heat treated sample was centrifuged for 10 minutes at 10K×g. Supernatant was transferred to a clean tube. 40% PEG (mw 8000) was added to a final [ ] of 4% and 5M NaCl was added to a final [ ] of 0.6 M. Sample was mixed well by inverting, and place on ice for at least 45 minutes. Virus was collected by centrifuging for 10 minutes at 12K×g. Virus pellet was resuspended in approximately 1/10th original extract volume of 50 mM Phosphate buffer, pH 7.2. Sample was centrifuged for 10 minutes at 12K×g to clarify, and the supernatant saved.

After virus purification, RNA was purified from the pooled virus prep using Qiagen RNAeasy kit, according to manufacturers instructions. The CP gene was amplified from the viral RNA sample using the Promega ImPromII RT-PCR kit as follows: One microgram of purified RNA was primed with 10 pmoles JAL 619 oligo (GCCTTGGTACCTGGGCCCCTACCGGGGGTAACGG). Two microliters of RT reaction were used in a PCR reaction using oligos JAL 619 and JAL 618 (cgatgatgattcggaggctactg) which sits about 300 bp upstream of the U1 CP start codon. The resulting PCR product was digested with NcoI and KpnI the fragment containing the CP orf isolated from an agarose gel and ligated into NcoI-KpnI digested pLSB 1298.

The ligation reaction was transformed into DH5a E. coli and plated on LB-Agar plates containing 100 ug/ml ampicillin. DNA was purified from cultures from individual colonies and analyzed by DNA sequence analysis using JAL 153 (aggctactgtcgccgaatc) as a sequencing primer. The deduced amino acid sequence of some of the CPs genes cloned from this experiment are presented in Table 5.4 Select clones were transcribed using Ambion's T7 mMessage mMachine kit, encapsidated in U1 CP, as described above, and inoculated onto at N. benthamiana plants.

TABLE 5.4

CP amino acid sequences of select 2901
isolates.

| | |
|---|---|
| Isolate Name | CP seq |

| | |
|---|---|
| U1 CP | MSYSITTP . . . WTSGPAT |
| pLSB 2901.1 | MASYSITTP . . . WTS ACL GPAT |
| pLSB 2901.3 | MASYSITTP . . . WTS DCC GPAT |
| pLSB 2901.4 | MASYSITTP . . . WTS LCP GPAT |
| pLSB 2901.6 | MASYSITTP . . . WTS PCP GPAT |
| pLSB 2901.7 | MASYSITTP . . . WTS TCA GPAT |
| pLSB 2901.13 | MASYSITTP . . . WTS QCP GPAT |
| pLSB 2901.16 | MASYSITTP . . . WTS VCL GPAT |
| pLSB 2901.23 | MASYSITTP . . . WTS SCR GPAT |

### EXAMPLE 11

### Mitigating Library for the HIV V3 Loop Sequence in the 60s Loop Region of U1 CP

### pLSB 2902 Library

Fusion of HIV V3 loop coding sequence to U1 CP gene at the loop or GPAT position.

### Materials and Methods:

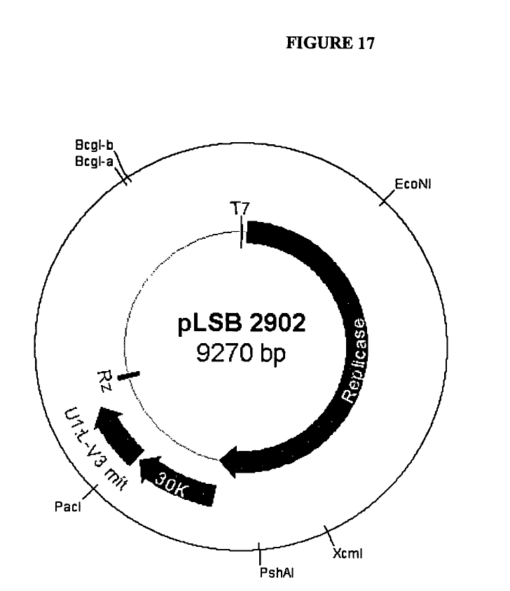

Using PCR based strategy a 123 bp insert consisting of the coding sequence for the HIV V3 loop sequence and three randomized (VNN) codons on either side of the HIV coding sequence. This DNA sequence was inserted, in frame, into the U1 CP gene coding sequence at either the “60s loop” region of the CP (library pLSB 2902) or in the “GPAT” position of the U1 CP sequence (PLSB 2903 library). (FIG. 17 pLSB 2902 Vector Map). FIG. 16 is the nucleotide sequence of CP fusion in vector 2902. Inserted sequence is in bold typeface.

Results: Virus was purified from plants inoculated with transcripts from the pLSB 2902 library, and analyzed by SDS PAGE. No CP fusion of the approximate estimated size was identified. RNA was extracted and analyzed by RT-PCR using primers that sat at the 3′ end of the virus (R primer) and at the CP start codon (F primer). The only PCR product obtained was the size of wt CP (i.e. NO insert in the loop region).

Using a modified RT-PCR screening method, RNA was then primed with a primer that sat at the virus 3′ end. PCR was then performed with one oligo that would anneal to the V3 loop insert sequence, and a second oligo that annealed to TMV sequences. Using this approach, a PCR product the size of a CP fusion containing the V3 loop insert sequence was obtained. The product was digested with NcoI and KpnI and cloned into a TMV vector backbone. The resulting transformants were analyzed by DNA sequence analysis (to confirm the presence of a V3 loop sequence). Isolates of interest were transcribed with T7 RNA polymerase and transcripts inoculated onto N. benthamiana. Several days post inoculation N.b. plants began showing systemic symptoms. However, no virus could be purified from the systemically infected tissue.

My interpretation of these combined results is that transencapsidation occurred between a virus that had the V3 loop insert in its CP gene, and a virus that had deleted all or a portion of the insert. This resulted in virions containing V3 loop inserts being purified in the initial library passage portion of the experiment. I think many of these viruses with V3 loops in the CP do not produce functional CP, but they can move as free RNAs to infect systemic tissue, and they can be transencapsidated by functional CP produced from another virus.

An alternative way to analyze this library would be to use antisera specific for the V3 loop to pull down any viruses that have this CP fusion. Then RT-PCR clone, sequence, etc. these clones.

### EXAMPLE 12

### Mitigating Library for the HIV V3 Loop Sequence at the GPAT Position of U1 CP

### pLSB 2903 Library

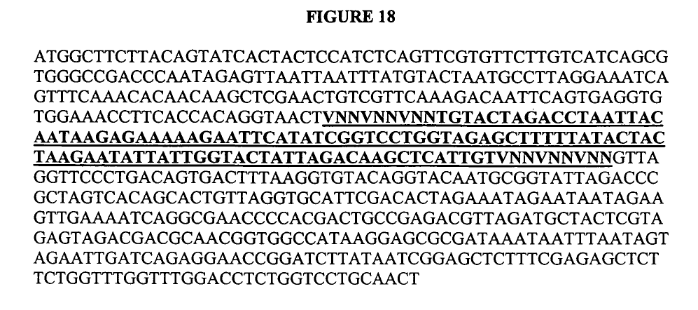

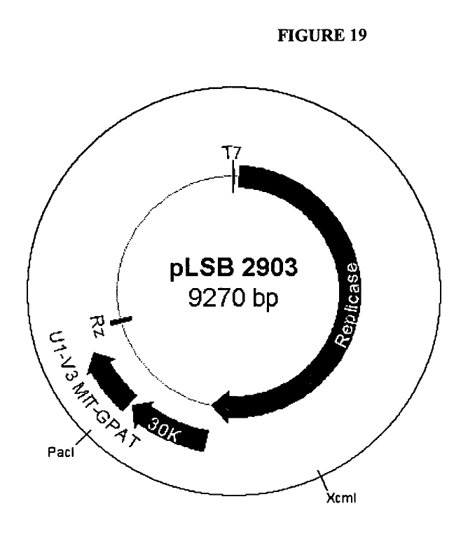

Using PCR based strategy a 123 bp insert consisting of the coding sequence for the HIV V3 loop sequence and three randomized (VNN) codons on either side of the HIV coding sequence. This DNA sequence was inserted, in frame, into the U1 CP gene coding sequence at either the “60s loop” region of the CP (library pLSB 2902) or in the “GPAT” position of the U1 CP sequence (pLSB 2903 library). FIG. 19 pLSB 2903 Vector Map. FIG. 18 Nucleotide sequence of CP fusion in vector 2903. Inserted sequence is in bold typeface.

### Results:

N. benth plants inoculated with this library went systemic. Virus was purified from systemically infected tissue. However, when analyzed by SDS PAGE, No CP fusion was found.

RNA was extracted from the virus, and RT-PCR of the CP/3 UTS was performed. The resulting PCR product was cloned and sequenced. The RT-PCR products sequenced showed that at least part of the V3 loop sequence still remained. However, stop codons/frameshifts prevented the v3 loop sequence from being translated. Thus it seemed “easier” for the virus to simply generate premature stop codons, than to throw out this extra sequence at the 3′ end of the CP ORF.

Again, an alternative selection procedure, using anti V3 antisera to pull down viruses with V3 epitope in the CP would probably be a good thing to try.

### EXAMPLE 13

### Mitigating Library for the LQN Epitope of HIV in the 60s Loop Region of U1 CP

### pLSB 2904 Library

LQN in Loop Library (pLSB 2904 Library)

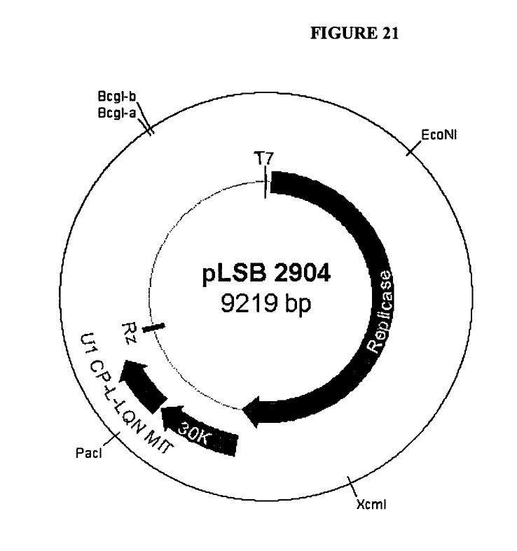

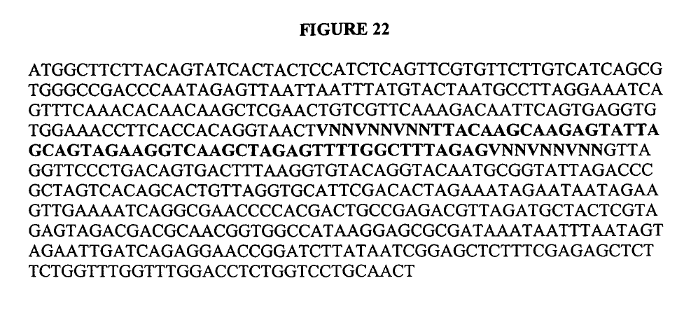

Using PCR based insertional mutagenesis/sequence overlap extension PCR the coding sequence for the LQN epitope, with three randomized flanking codons on each side was inserted into the “60s loop” region of the U1 CP. See FIG. 1 for nucleotide sequence of the CP in pLSB 2904 library. Oligos JAL 650 and JAL 649 were used to construct the LQN epitope with degenerate flanking codons. FIG. 21 pLSB 2904 Vector Map. FIG. 22 Nucleotide sequence of CP fusion in vector 2904. Inserted (non-TMV CP sequence) is in boldface.

A library of about 2800 colonies in size, was pooled from agarose plates and DNA prepared from the pooled clones. DNA was transcribed with Ambion's T7 mMessage mMachine transcription kit, according to manufacturers instructions. RNA was packaged in purified TMV CP and encapsidated RNA inoculated onto N. benthamiana plants. About 8 DPI one systemically infected leaf of each plant was harvested. All leaves were combined and virus purified from the pool of leaves using pH (5.0)/heat (50° C.) purification strategy. Briefly, tissue was frozen with liquid nitrogen, ground to a powder with a mortar and pestle, then ground in the presence of 3 volumes pH 5.0, 50 mM acetate buffer. Sample was filtered thru cheesecloth, held at 50 C for 5 minutes and then centrifuged at 10 k×g for 10 minutes. Clarified supernatant was saved, and PEG and NaCl added to final [ ] of 4% and 0.6 M, respectively. After adding PEG and NaCl sample was held on ice at least 1 hour, then centrifuged for 15 min at 12K×g. Pellet (containing virus) was resuspended in 1/10th volume original extract buffer of 50 mM phosphate pH 7.0. Sample was centrifuged 10 minutes at 12 k×g and supernatant (containing virus) was saved in a clean tube.

Note: The original pLSB 2904 library constructed was screened by PCR. The results were somewhat ambiguous suggesting that the pLSB 2904 library (prepared in mid June 2004) did not have a single sized CP fusion. As a result of this (and other results, such as small library size, no CP fusions obtained in first passage through plants, etc.) I decided to reconstruct this library. Construction of a new pLSB 2904 library is ongoing as of Jul. 16, 2004.

### EXAMPLE 14

### Mitigating Library for the HIV “4E10” Epitope at the N Terminal End of U1 CP

### pLSB 2905 Library

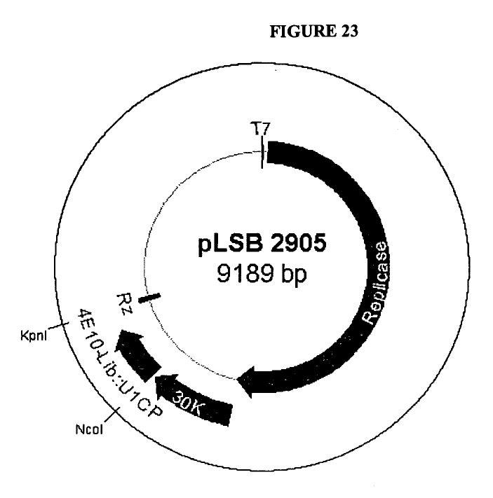

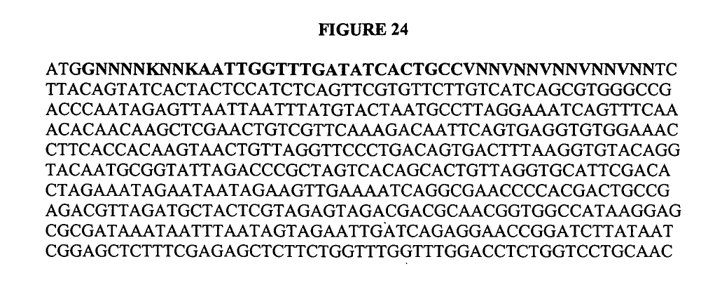

Using a PCR based strategy and mutagenic oligos JAL 627 and JAL 629 library pLSB 2905 was constructed. The nucleotide sequence of the recombinant CP gene sequence in pLSB 2905 library is shown in FIG. 23, 24: FIG. 23. pLSB 2905 Vector Map. FIG. 24 Nucleotide sequence of CP fusion in vector 2905. Non-TMV sequence is in bold. DNA was prepared from a library of about 25 to 30K colonies

Library was transcribed (Ambion T7 mMessage mMachine kit), encapsidated in purified U1 CP and inoculated onto 1 “glurk” plant leaf, and about 40 N. benths. Inoculation date Jul. 15, 2004. Virus was purified from systemically infected N. benth tissue. RNA extracted from virus and amplified by RT-PCR with oligos JAL 618 (F) and JAL 619 (R) oligos. PCR product was digested with NcoI and KpnI and ligated into NcoI-KpnI digested pLSB 1298 vector.

### EXAMPLE 15

### Mitigating Library for the HIV “4E10 Long” Epitope at the N Terminal End of U1 CP

### pLSB 2907 Library

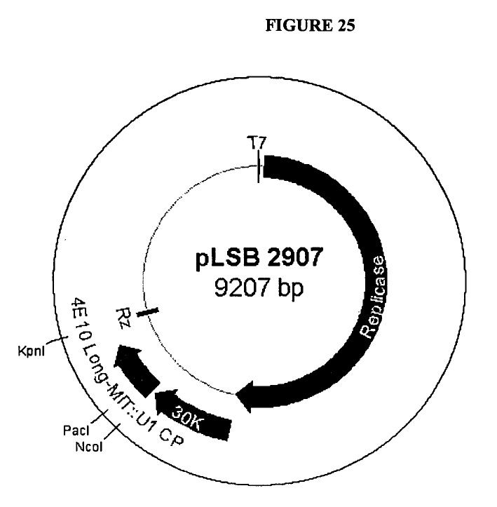

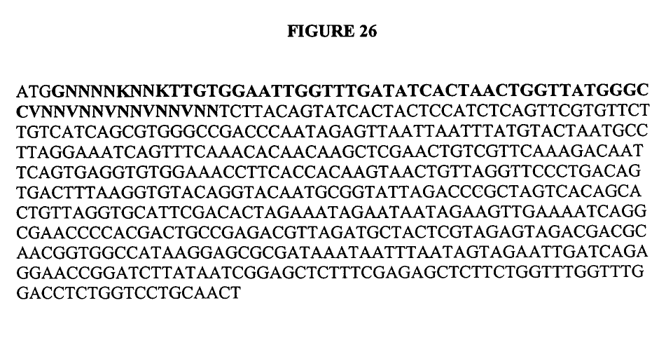

Using PCR based insertional mutagenesis and oligos JAL 630 and JAL 627 the coding sequence for the HIV “4E 10 long” epitope (LWNWFDITNWLWA) and degenerate flanking codons was constructed and fused to the 5′ end of the U1 CP gene. FIG. 25 pLSB 2907 Vector Map. FIG. 26 Nucleotide sequence of CP fusion in vector 2907. Non-TMV sequences are in bold.

Ligation of pLSB 2907 library is described in in JAL notebook 34, LSBC Notebook # 1412. Ligation transformed into E. coli Jul. 16, 2004.

Virus purified from infected N. benth tissue. (limsbo 105-36 or 105-65). RNA extracted from virus, RT pcr. Selection based PCR of “upper half” of CP fusion with 30B5522F oligo and JAL 665 R oligo (about 300 bp product). “lower half” of CP fusion was amplified with JAL 664 (F) and JAL 619 (R) oligos (ca. 700 bp product). 700 and 300 bp PCR products were joined by sequence overlap extension PCR.

SOE product was either re-amplified with oligos JAL 618 and 619 or cloned directly. In either case, PCR products were digested with NcoI and KpnI and ligated into NcoI KpnI digested pLSB 1298 vector. Limsbo 105-61 and 105-63 are samples of the cloning of ‘2907’ selected CP. NOTE: the efficiency of cloning of these 2 ligations may differ. Test each one (by restriction enzyme digestion of several transformants of each library) to identify the library with the highest cloning efficiency (ie highest % age of clones with proper sized insert).

### EXAMPLE 16

### Mitigating Library for the HIV “2F5 Long” Epitope at the N Terminal End of U1 CP

### pLSB 2908 Library

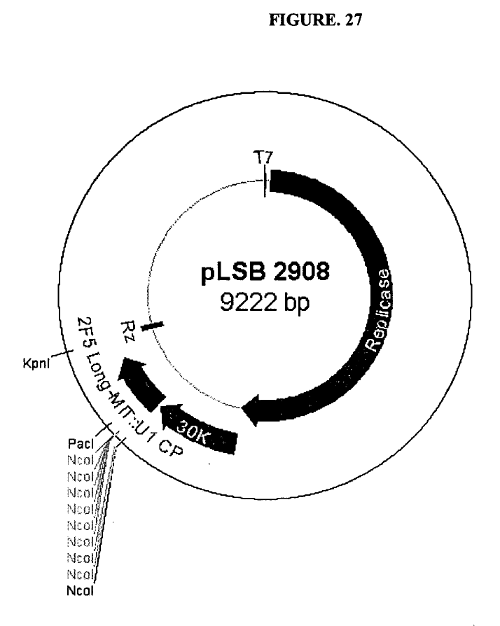

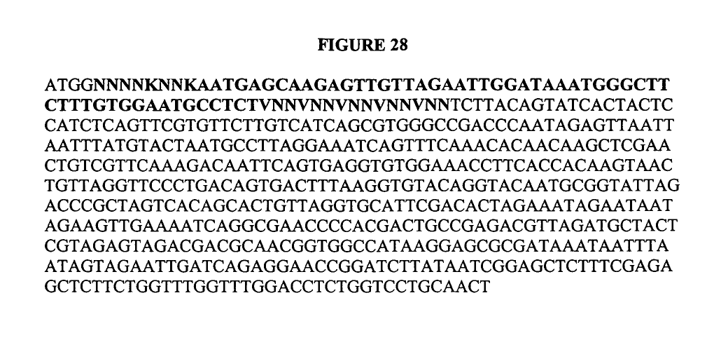

pLSB 2908 library: Fusion of “2F5 long-mitigating sequence” library on U1 CP N terminus in pLSB 1298 vector backbone. Using a PCR based strategy for insertional mutagenesis, oligos JAL 631 and JAL 628 and sticky RICE the coding sequence for the “2F5 long” epitope of HIV (NEQELLELDKWASLWN) flanked by 3 degenerate codons was fused to the 5′ end of the U1 CP coding sequence. For sequence of the CP fusion in the 2908 library, see FIGS. 27,28. FIG. 27 pLSB 2908 Vector Map. FIG. 28 Nucleotide sequence of CP fusion in vector 2908. Inserted nucleotide sequences are in bold.

Virus purified from infected N. benth tissue. (limsbo 105-37). RNA extracted from virus, RT pcr. Selection based PCR of “upper half” of CP fusion with 30B5522F oligo and JAL 663 R oligo (about 300 bp product). “lower half” of CP fusion was amplified with JAL 662 (F) and JAL 619 (R) oligos (ca. 700 bp product). 700 and 300 bp PCR products were joined by sequence overlap extension PCR.

SOE product was either re-amplified with oligos JAL 618 and 619 or cloned directly. In either case, PCR products were digested with NcoI and KpnI and ligated into NcoI KpnI digested pLSB 1298 vector. Limsbo 105-62 and 105-64 are samples of the cloning of ‘2907’ selected CP. NOTE: the efficiency of cloning of these 2 ligations may differ. Test each one (by restriction enzyme digestion of several transformants of each library) to identify the library with the highest cloning efficiency (i.e. highest % age of clones with proper sized insert).

### EXAMPLE 17

### Generation of Soluble Tobacco Mosaic Virus Displaying a Surface Exposed Lysine

The amino terminus of the TMV coat protein (CP) resides on the exterior of TMV particles, allowing amino acids fused to the N-terminus of the TMV CP to be displayed on the virion surface. Using standard molecular techniques, codons for the amino acid sequence glycine-lysine-glycine-alanine-glycine (GKGAG), were fused to the 5′ end of the TMV CP ORF in a plasmid containing the full-length cDNA clone of TMV RNA, under the control of the T7 RNA polymerase promoter. The resulting plasmid was named pLSB 2800. T7 transcripts of pLSB 2800 were inoculated onto Nicotiana benthamiana plants to generate LSB 2800 virion. (Recombinant virus names are based on the names of the plasmid-based cDNA clones. This same nomenclature is followed throughout this manuscript). Recombinant virus LSB 2800 was purified from systemically infected N. benthamiana plants, by a modification of the procedure of Gooding and Herbet (1967). Briefly, the tissue was homogenized in 0.86 M NaCl, 0.04% w/v sodium metabisulfite (0.5 g of tissue/ml of buffer), adjusted to pH 5.0, heated to 47° C. for 5 minutes, chilled and centrifuged at 6,000×g for 5 minutes. The clarified supernatant was subjected to two sequential PEG precipitations to recover the virus. This concentrated virus was extracted with 25% chloroform v/v, to remove host protein impurities and pigments that remained associated with the virus.

The LSB 2800 virus aggregated with storage (see Results), so a library-based strategy was undertaken, to construct a recombinant TMV CP which would have both a surface exposed lysine residue and high solubility. A full length cDNA clone of the wild type U1 strain of TMV under the control of the T7 RNA polymerase promoter (Dawson et al., 1986) was modified to contain a unique NcoI site at the ATG codon of the coat protein ORF, and a unique KpnI site at the 3′ end of the viral cDNA sequence. This plasmid (pJL 150/254) was used as the base vector for construction of a library of TMV CP mutants. Using a PCR based approach, three randomized codons and a lysine codon were fused to the 5′ end of the U1 CP ORF. The TMV CP gene and 3′ UTR were amplified from a full-length cDNA clone of the U1 strain of TMV (p801) using oligonucleotides JAL 613 (CGAACCATGGNNNNKNNKAAATCTTACAGTATCACTACTCCATCTCA) as a forward primer and JAL 590 (GCCAACACATCCGGGTACCTGGGCCCCTA) as a reverse primer. The resulting PCR product of about 800 bp was digested with NcoI (underlined in JAL 613 sequence) and KpnI (underlined in JAL 590 sequence) and ligated into NcoI, KpnI digested pJL 150/254 to produce the CP library, pLSB 1295. The pLSB 1295 library was transformed into DH5□ Escherichia coli and plated. DNA was prepared from a pool of about 3600 colonies of this library, linearized with KpnI and transcribed with T7 RNA polymerase using the T7 mMessage mMachine® kit (Ambion, Austin, Tex.), according to manufacturer's instructions. Transcripts were encapsidated in purified TMV CP (Fraenkel-Conrat, 1957). A portion of the encapsidated RNA was used to inoculate the local lesion host N. tabacum cv. Xanthi (N). Encapsidated transcript was also inoculated onto 12 N. benthamiana plants at a concentration of approximately 100 infectious units per plant.

Approximately 8-10 days post-inoculation one systemically infected leaf from each N. benthamiana plant was harvested and pooled. TMV particles were purified from the pooled infected leaves as described above, but extracting in a buffer lacking NaCl and omitting the final chloroform treatment. Ten-fold dilutions (from 10⁻¹ to 10⁻⁴) of the purified virus pool were prepared. Four N. benthamiana plants were inoculated with 20 ul of each dilution. Approximately 8 days post inoculation, one systemically infected leaf of each plant was collected and pooled. Virus was purified from the pooled leaves, diluted and re-passaged onto N. benthamiana plants (as described above) for two additional cycles. After the final virus purification, RNA was purified from the passaged virus preparation using the Qiagen RNAeasy kit (Valencia, Calif.), according to the manufacturers instructions. The CP gene was amplified from 10 g of purified viral RNA using the Promega ImPromII RT-PCR kit (Madison, Wis.) and the oligonucleotide JAL 619 (GCCTTGGTACCTGGGCCCCTACCGGGGGTAACGG). The coat protein gene region and 3′ UTR was subsequently amplified from the RT reaction product using oligonucleotides JAL 618 (CGATGATGATTCGGAGGCTACTG) and JAL 619 as forward and reverse primers, respectively. JAL 619 anneals to the very 3′ end of the TMV ORF, and JAL 618 anneals approximately 300 bp upstream of the U1 CP start codon. The resulting PCR product was digested with NcoI and KpnI, the fragment containing the CP ORF gel purified and ligated into NcoI-KpnI digested pJL 150/254. This ligation reaction was transformed into DH5□ E. coli and DNA samples from individual colonies were sequenced to determine the coding sequence at the 5′ end of the CP gene of individual isolates of the selected library. To obtain virus samples of recombinant clones from the selected library, plasmid clones of individual library members were linearized with KpnI, transcribed with T7 RNA polymerase and transcripts inoculated onto N. benthamiana plants. TMV particles were purified from systemically infected tissue as described earlier. Individual virus samples were analyzed by protein gel electrophoresis and mass spectrometry to confirm the identity of the additional amino acids on the CP N-terminus.

### EXAMPLE 18

### Cloning, Expression and Purification of the GFP-Streptavidin Fusion

Purified Streptomyces avidinii genomic DNA was obtained from ATCC (Manassas, Va.) and the streptavidin core (SA) coding sequence (ca. 500 bp) was amplified by PCR. The SA coding sequence was ligated to the SacI site in the GFP insert in the TMV-expression vector, p³⁰B GFPc3 (Shivprasad et al., 1999), using standard cloning techniques. Infectious transcripts from the resulting plasmid, pLSB 1290, which expressed a fusion of GFP to the N-terminus of the streptavidin core. In vitro transcripts derived from pLSB 1290 were inoculated onto N. benthamiana plants.

To isolate the GFP-SA fusion, infected plant tissue was homogenized in a Waring blender (Torrington, Conn.), in three volumes of extraction buffer (100 mM phosphate pH 7.2, 0.01% Na-metabisulfite, 1 □l BME per ml) and filtered through cheesecloth, to obtain an initial “green juice” (GJ) extract. The GJ was heated to 52-55° C. for 7 minutes, centrifuged at 12000×g for 10 minutes and TMV removed from the clarified supernatant by precipitation with 25% ammonium sulfate. The GFP-SA containing protein fraction was precipitated with 50% ammonium sulfate, resuspended to ⅕^{th} the original volume, and dialyzed against 1× phosphate buffered saline (PBS), adjusted to pH 9.3. Affinity chromatography was performed using an AKTA purifier 10 system (Amersham Biosciences, Piscataway, N.J.). The clarified plant extract was adjusted to pH 11, 0.2 □m filtered and loaded at 1 ml/min onto an immobilized iminobiotin resin (Pierce, Rockfold, Ill.) in HR5/10 column format (Amersham Biosciences), using 1 ml resin per 20 g tissue extracted. The captured GFP-streptavidin fusion was eluted using 0.1 M acetic acid, pH 4.0 and the peak fractions combined with an equal volume of 250 mM phosphate buffer, adjusted to pH 10.5. Purified GFP-SA was subsequently dialyzed against 25 mM phosphate buffer, pH 9.3 and placed at 4° C., or −20° C. for extended storage.

Biotinylation of the LSB 1295.4 rTMV Surface-Exposed Lysine

From the library passage experiment, multiple unique recombinant viruses were obtained (Table 1) which remained soluble post purification at concentrations of 10 mg/ml or higher. TMV recombinant LSB 1295.4 (from pLSB 1295.4) containing the N-terminal amino acids ADFK was selected for in vitro biotinylation reactions. Biotin was covalently conjugated to the surface-exposed lysine using EZ-Link NHS-PEO₄-Biotin (Pierce, Rockfold, Ill.). Lyophilized NHS-PEO₄-Biotin, was dissolved in phosphate buffer containing the rTMV at a concentration of 0.8 mg/ml, with the biotin analog typically present at a 24-fold or 240-fold molar excess. The conjugation reactions were incubated with mixing at either 4° C. or room temperature, for up to 60 hours, as described in the Results. Unreacted NHS-PEO₄-Biotin was removed by PEG precipitation of the biotinylated rTMV (4% w/v PEG, 4% w/v NaCl). The precipitated virus pellet was washed and was resuspended in 1×PBS to a final TMV concentration of 10 mg/ml. Analytical procedures, immunoassays and electron microscopy

Polyacrylamide gel electrophoresis (PAGE) of proteins in the presence of sodium dodecyl sulfate (SDS) (Laemmli, 1970) was performed on 10-20% Tris Glycine gels (BioRad, Hercules Calif., or Invitrogen, Carlsband, Calif.), following the manufacturer's instructions. The Mark 12 or Magic Mark XP proteins standards (both Invitrogen) were employed as molecular weight references. For Western blots, proteins were transferred electrophoretically (Towbin, Staehelin, and Gordon, 1979) to a 0.20 m polyvinylidene fluoride membrane (BioRad) and stored overnight in blocking buffer (Tris-buffered saline (pH 8.0) 0.05% Tween (TBST) containing 5% w/v dry milk). For coat protein detection, blots were probed with the rabbit anti-TMV polyclonal, PVAS-135D (ATCC) and to detect the GFP-SA fusion a rabbit anti-GFP polyclonal (Polysciences, Warrington, Pa.) was employed. In both cases, the secondary antibody was a goat anti-rabbit horseradish peroxidase (HRP) conjugate (BioRad). To evaluate rTMV biotinylation, blots were probed with either streptavidin HRP (Pharmingen, San Diego, Calif.) or the GFP-SA fusion. HRP detection was with the ECL+chemiluminescent kit (Amersham Biosciences, Buckinghamshire, England) with Hyperfilm ECL (Amersham) employed for image capture, as per the manufacturer's instructions.

Amino acid analysis (AAA) was performed by the Molecular Structure Facility at the University of California, Davis. Protein quantitation was also performed using the bicinchoninic acid (BCA) assay (Pierce) in a microtiter plate format following the manufacturer's instructions. Wild-type TMV, type U1, quantified by AAA was employed as a standard. Protein mass determination was performed by matrix-assisted laser desorption ionization time-of-flight mass spectrometry (MALDI-TOF MS) using a Voyager-DE™ STR Biospectrometry™ Workstation (Applied Biosystems, Foster City, Calif.). The mass spectrometer was operated in linear and positive mode using delayed extraction at an accelerating voltage of 25 kV and 128 laser shots per spectrum. Protein samples were diluted in sinipinic acid (10 mg/mL prepared in acetonitrile:0.1% TFA, 1:2, v/v) and spotted onto a MALDI-TOF target plate for analysis. A single point mass calibration was performed using horse heart apomyoglobin (Sigma, St. Louis, Mo.). All MALDI-TOF sample spotting was performed using a dried-droplet method (Karas and Hillenkamp, 1988). All raw spectral data were processed using Applied Biosystems Data Explorer 4.0.0.0. Protein identification was determined using General Protein/Mass Analysis for Windows (GPMAW), version 5.0 (Lighthouse Data, Denmark) software.

For electron microscopy, grids (400 Mesh copper, carbon coated; Ted Pella, Bedding, Calif.) were floated on drops of 1×PBS, pH 9.5 containing rTMV or the rTMV GFP-SA complex, diluted to a concentration of 100-200 μg/ml (relative to the rTMV alone). Following a 15 minute contact time, excess liquid was removed, the grids negatively stained with 1% phosphotungstic acid (PTA) and permitted to air dry. Bacitracin at 25 □g/ml was employed during both the sample coating of the grid and negative staining (Gregory and Pirie, 1973). All samples were examined on a Philips CM120 microscope, coupled to a Gatan MegaScan 795 digital camera.

### EXAMPLE 19

### Manufacture of the rTMV GFP-Streptavidin Complex

Affinity purified GFP-SA was concentrated to approximately 2 mg/ml using a Savant speed-vac (Framingdale, N.Y.) and combined with a 10 mg/mil biotinylated rTMV stock, to obtain a final GFP-SA to rTMV CP mass ratio of 3:1. This mixture was incubated overnight at room-temperature with gentle agitation to allow the GFP-SA fusion to bind to the biotinylated virion. Unbound GFP-SA was removed by precipitation of TMV particles with 4% PEG (MW 6000), 4% NaCl and centrifugation for 15 minutes at 10,000×g. The supernatant, containing excess GFP-SA was discarded and the pelleted rTMV/GFP-SA complex resuspended to the starting volume in 25 mM phosphate buffer, pH 9.5. The PEG precipitation steps were repeated twice, with the resuspended rTMV/GFP-SA complex transferred to a new centrifuge tube prior to the final precipitation. In parallel, a control incubation employing unbiotinylated rTMV was performed. Siliconized centrifuge tubes and pipettes were employed during the manufacturing. The final rTMV/GFP-SA complex and the control incubation were analyzed by AAA to determine the complex composition.

### Immunogenicity Testing in Mice and Guinea Pigs

The immunogenicity of the rTMV/GFP-SA complexes was evaluated in both mice and guinea pigs. The murine studies were performed using 6-8 week old female BALB/c mice (Harlan Sprague Dawley, Indianapolis, Ind.). Mice received 2 immunizations, administered subcutaneously at 2-week intervals in the absence of adjuvant. Tail-bleeds were taken nine days after each injection. All dosing was normalized to the mass of GFP administered, with animals receiving either 1 □g or 10 □g GFP (1.3 □g or 13 □g GFP-SA respectively). Groups of five mice were employed and the high and low responders from each group were omitted in the analysis. For the guinea pig study, 20 week old animals (Harlan Sprague Dawley) (2 per group) received a total of two 10 □g GFP immunizations delivered at four sites along the back. For half the groups, the antigens were combined with the adjuvant RIBI (Corixa, Hamilton, Mont.), as per the manufacturer's instructions. Antigen was administered at 4 week intervals, with bleeds taken two weeks after each injection by intravenous puncture of the cranial vena cava. Table 2 summarizes the antigen groups evaluated for both the murine (M) and guinea pig (GP) studies.

### Serological Analysis of Immune Response

Antibody response to antigen was determined by enzyme linked immunosorbent assay (ELISA). 96-well microtiter plates (MaxiSorp; Nalge Nunc, Rochester, N.Y.) were coated overnight at 4° C. with GFP (TMV-vector expressed and purified to >90%), diluted to 0.5 ug/mL in carbonate/bicarbonate buffer (pH 9.6). After blocking with BSA (2% w/v in 100 mM Tris (pH 7.5), 500 mM NaCl, containing 0.1% v/v Tween 20), sera diluted in 1×PBS with 2% w/v BSA was added and a three-fold serial dilution performed, starting at an initial dilution of either 1:25 (bleed 1, mice) or 1:150 (all others). Following a one-hour incubation at 37° C., the plates were washed and incubated for an additional hour with the appropriate HRP conjugated secondary antibodies; goat-anti mouse IgG (Southern Biotechnology Associates, Birmingham, Ala.) or goat-anti-guinea pig IgG (Bethyl Laboratories, Montgomery, Tex.). Plates were developed using a 3,3′, 5,5′-tetramethyl bezidine substrate solution (Turbo TMB ELISA; Pierce) and the reactions stopped by the addition of IN sulfuric acid. Plate absorbance was read at 450 nm in a 96-well plate spectrophotometer (Molecular Devices, Sunnyvale, Calif.). The serum endpoint dilution was taken as the highest dilution at which the absorbance reading was at twice background. Data and statistical analysis was performed using GraphPad (version 4.0) (San Diego, Calif.)

TABLE 1

Deduced amino acid sequence, frequency and
virion yield for a selection of pLSB1295
library isolates.

| | | | |
|---|---|---|---|
| pLSB | Sequence | Frequency | Yield mg/g FW |

| | | | |
|---|---|---|---|
| 2800 | M GKG SYS | — | 0.1-0.2 |
| 1295.1 | M AEFK SYS | 4 | 4.3 |
| 1295.2 | M EVLK SYS | 2 | ND |
| 1295.3 | M AEVK SYS | 13 | 1.3 |
| 1295.4 | M ADFK SYS | 27 | 5.2 |
| 1295.6 | M DVEK SYS | 2 | 2.0 |
| 1295.9 | M EGAK SYS | 7 | 0.9 |
| 1295.10 | M EPMK SYS | 1 | 7.3 |
| 1295.11 | M EMGK SYS | 4 | 4.3 |
| 1295.12 | M DGAK SYS | 1 | 2.7 |
| 1295.13 | M EGLK SYS | 13 | ND |
| 1295.14 | M VEFK SYS | 2 | ND |

TABLE 2
Experimental set-up for animal studies to evaluate
GFP-SA/rTMV complex immunogenicity.

| | | Adju- | GFP | GFP-SA | rTMV |
|---|---|---|---|---|---|
| | Antigen | vant | dose | dose | dose |
| M | PBS | No | — | — | — |
| M | GFP-SA alone | No | 1 μg | 1.3 μg | — |
| | (GFP SA) | | | | |
| M | GFP-SA + unbiotinylated rTMV | No | 1 μg | 1.3 μg | 0.57 μg |
| | (I GFP SA) | | | | |
| M | GFP-SA/rTMV complex | No | 1 μg | 1.3 μg | 0.57 μg |
| | (Bt GFP SA) | | | | |
| M | GFP-SA alone | No | 10 μg | 13 μg | — |
| M | GFP-SA + unbiotinylated rTMV | No | 10 μg | 13 μg | 5.7 μg |
| M | GFP-SA/rTMV complex | No | 10 μg | 13 μg | 5.7 μg |
| GP | GFP-SA + unbiotinylated rTMV | No | 10 μg | 13 μg | 5.7 μg |
| GP | GFP-SA + unbiotinylated rTMV | Yes | 10 μg | 13 μg | 5.7 μg |
| GP | GFP-SA/rTMV complex | No | 10 μg | 13 μg | 5.7 μg |
| GP | GFP-SA/rTMV complex | Yes | 10 μg | 13 μg | 5.7 μg |

Species;
M, BALB/c mice;
GP, guinea pigs.

### EXAMPLE 20

### Generation of a Soluble and High Yielding Lysine TMV Coat Protein Fusion

Initially, to obtain a surface exposed lysine, we engineered a rTMV that expressed a CP with the amino acids GKGAG fused to the N-terminus (LSB 2800). However, this recombinant virus was suboptimal for our studies; the yield of purified virion (FIG. 1A) was low, at 0.1-0.2 mg/kg infected tissue and an organic extraction was required, to remove green pigment that remained associated following rTMV precipitation by polyethylene glycol (PEG). In addition, this construct aggregated upon storage at 4° C. (FIG. 1 B). To circumvent these problems, a library based passage and selection scheme was employed, to select for viruses displaying a surface exposed lysine that retained solubility at high protein concentrations. A lysine was introduced at the N-terminus of the type U1 strain of TMV, with three randomized codons placed immediately upstream. Transcripts from approximately 3600 clones were pooled and inoculated onto N. benthamiana plants. Isolation of virion from systemically infected tissue selected for functional library members possessing the desired purifications characteristics. This virion pool underwent two additional rounds of selection on N. benthamiana. Between these selection rounds the virus was stored for 2-5 days at 4° C., at a concentration of 10 mg/ml or greater, to permit virion with a propensity for aggregation to settle and the supernatant employed in subsequent virus passages. A total of 74 isolates from this selected library were sequenced and 11 unique coat protein fusions identified (Table 1). All isolates had at least one acid residue (glutamic acid or aspartic acid) one or two residues N-terminal of the fixed lysine residue. A single isolate was obtained (PLSB 1295.6) where two acid residues were introduced, one being immediately N-terminal to the lysine residue. Together this small collection of selected viruses suggests that one or more surface exposed acidic residues were needed to mitigate the presence of the surface exposed lysine. Eight of these individual isolates were transcribed and inoculated onto plants to compare yields to those obtained from LSB 2800 (Table 1). The LSB 1295 library represented a 10 to 70-fold yield improvement over LSB 2800, with recoveries exceeding 7 mg/kg infected tissue noted for one isolate (LSB 1295.10). Similar to wild-type U1, no green pigment was associated with the purified LSB 1295 virion, obviating the need for the organic extraction step. On the basis of expression level and isolate frequency, LSB 1295.4, with the amino acid sequence ADFK at the CP N-terminus, was carried forward. This recombinant virion derived from the evolution and selection process (FIG. 1A) exhibited improved solubility characteristics relative to LSB 2800; with 4° C. storage at 13 mg/ml, no precipitation was observed after 10 days (FIG. 1B).

### EXAMPLE 21

### GFP-Streptavidin Expression in Planta

TMV-vector expression of the GFP-SA fusion in N. benthamiana plants did not affect plant growth or biomass accumulation, with the expected mosaic phenotype observed on systemically infected tissue approximately 6 days post inoculation. Viral movement, assessed by GFP fluorescence, was retarded relative to vectors expressing GFP alone, although near confluent fluorescence of the systemically infected tissue occurred 8-9 days following inoculation. The GFP-SA fusion was detectable in crude plant extracts (FIG. 1C, GJ lane), migrating as a single band with an apparent molecular weight of 40 kDa and of comparable intensity to the TMV coat protein band, which migrated at 21 kDa. Differential ammonium sulfate precipitation was employed to remove TMV and concentrate the streptavidin fusion, which was recovered by affinity chromatography at greater than 90% purity and the yield of purified fusion ranged from 60-120 mg/kg fresh weight processed. By MALDI-TOF mass spectrometry, the observed mass of the purified species was within 0.05% of 39717.1 Da, corresponding to the full-length GFP-SA fusion, with the methionine cleaved and the N-terminal amine acetylated (data not shown). The tetrameric form of core streptavidin is stable at 60° C. and below (Bayer, Benhur, and Wilchek, 1990). To confirm tetramerization of the GFP-streptavidin fusion, samples incubated at 60° C. or 95° C. in SDS-PAGE loading buffer were analyzed by gel electrophoresis (FIG. 1D). At 60° C. the prominent species migrated between 150 and 200 kDa, indicative of tetramer formation, while at 95° C., the 40 kDa fusion monomer predominated, with a minor ˜12 kDa species also present.

### EXAMPLE 22

### TMV Biotinylation and Complex Formation with GFP-Streptavidin

In vitro biotinylation of the surface exposed lysine of LSB 1295.4 was accomplished using the commercially available N-hydroxysuccinimide (NHS) PEO₄-biotin. LSB 1295.4 was combined with the NHS-PEO₄-biotin reagent at either a 24 or 240-fold molar excess relative to the viral coat protein and incubated for up to 48 hours at either room temperature or 4° C. Although biotin linker attachment to the 18 kDa CP represents only a 473 Da increase in molecular weight, it resulted in a discernable shift by gel electrophoresis (FIG. 2A) permitting the extent of coat protein conjugation to be assessed. With a 24 fold excess of biotin linker, approximately 20% of the virus surface was conjugated (FIG. 2A) and biotinylation was confirmed by mass spectrometry (FIG. 2B). With a further 10-fold increase in linker excess we achieved >98% biotinylation (FIG. 2A), which was reflected qualitatively in the MALDI-TOF MS spectrum by a reduction in peak intensity of the LSB 1295.4 CP relative to its conjugated counterpart (FIG. 2C). To obtain quantitative loading of the viral surface it was necessary to incubate the LSB 1295.4 virus preparation at room temperature in the presence of a 240-fold molar excess of the linker. Under these conditions we detected two additional minor bands by gel electrophoresis. This indicates conjugation of two or three biotins to a subset of the TMV capsid proteins. We also observed these minor species when we incubated type U1 coat protein with the biotin linker (FIG. 2A). The identity of the minor conjugation species was confirmed by mass spectrometry (data not shown). We performed western blot analysis to analyze the series of biotinylation reactions. For probing the western blots, we used either a streptavidin horseradish peroxidase conjugate or the TMV-vector expressed GFP-SA, and were able to detect the biotinylated U1 coat protein and the multiply conjugated LSB 1295.4 capsid protein with comparable efficiency with each of the SA-HRP conjugate, and the GFP-SA fusion protein probes (FIGS. 2D and E).

To array the GFP-streptavidin complex on the surface of the TMV, we used the virion preparations with quantitative biotin conjugation. The GFP-SA was present at a 1.3-fold molar excess relative to biotinylated coat protein, corresponding to a theoretical maximum loading of 710 tetramers per virion rod. Following incubation, the complex was isolated from unbound tetramer by sequential precipitation using either ammonium sulfate or PEG. Control reactions, employing unbiotinylated LSB 1295.4 demonstrated the specificity of the tetramer association (FIG. 3A). The complex recovered following PEG precipitation was examined by electron microscopy and compared to the starting virus scaffold, with and without biotinylation (FIG. 3B). The majority of the rods observed in the LSB 1295.4 virus preparation were of the expected length (300 nm) with a diameter of 18 nm. Complete biotinylation of the capsid proteins did not alter the size distribution of the virus. However, in the case of biotinylated TMV loaded with GFP-tetramer loading we observed a notable reduction in the average rod length: approximately 90% of rods were below 200 nm in length with 50-100 nm rods predominanting. A second electron dense layer, corresponding to the associated GFP-streptavidin was clearly visible, increasing the diameter of the rods to 29 nm (FIG. 3C). After extensive examination we could detect no partially coated or uncoated rods, suggesting complete coverage of the TMV rods. The complex was further characterized by gel electrophoresis and western blots (FIG. 3 D-F). Incubation at 60° C., in the presence of SDS-PAGE loading buffer, resulted in complete disassociation of the TMV virion, however the biotin/streptavidin linkage remained intact. The coat protein/tetramer association resulted in a band shift relative to the GFP-SA alone. With incubation at 95° C. the streptavidin/biotin linkage was broken and the GFP-SA and biotinylated coat protein migrated as monomers (FIG. 3 D). To confirm that the tetramer band shift observed at 60° C. was the result of biotinylated coat protein association, a western blot was performed, and an anti-TMV polyclonal was used as the probe (FIG. 3E). The bands that migrated at >220 kDa that represent the complex (Bt GFP SA) were detected and were also reactive when an anti-GFP (FIG. 3F) or anti-streptavidin (data not shown) polyclonal antibody was used as the probe. The control loading reactions, in which GFP-SA was combined with unbiotinylated LSB 1295.4 virus (I GFP SA) were analyzed in parallel. These samples were also subjected to sequential PEG precipitations. By gel electrophoresis (FIG. 3D) no GFP-SA was detected in the PEG precipitated control samples and this was confirmed by western blot (FIG. 3D). This indicates that the complex isolation procedure we employed effectively eliminated free SA fusion tetramers.

### EXAMPLE 23

### Quantitative Characterization of the GFP-Streptavidin/rTMV Complex by Amino Acid Analysis

The analysis described (FIG. 3) provides a qualitative characterization of the complex generated, but no information on the level of virion decoration by the GFP-SA. Initially, we used gel electrophoresis together with densitometric analysis of the Coomassie blue-stained bands to assess the extent of TMV loading. The intensity of staining by Coomassie brilliant blue is protein composition dependent, the principal interactions being with arginine residues (Compton and Jones, 1985). The GFP-SA fusion and the LSB 1295.4 CP each contain 11 arginine residues, therefore the coat protein has approximately twice the staining intensity per unit mass relative to the streptavidin fusion. However, the Coomassie dye also interacts with lysine, histidine and tyrosine residues (de Moreno, Smith, and Smith, 1986), preventing quantitation of the coat protein and GFP-SA entities using a single standard. Amino acid analysis was therefore investigated as an alternative. Since the full-length protein was determined to be the predominant species for both the LSB 1295.4 CP and the GFP-SA, the amino acid composition of the complex components can be related to the total pmoles of each residue detected, by a series of linear algebraic equations (Equation 1).

19U1ₚₘₒₗₑ+42GFP-SAₚₘₒₗₑ=ASXₚₘₒₗₑ

12U1ₚₘₒₗₑ+26GFP-SAₚₘₒₗₑ=LEUₚₘₒₗₑ

12GFP-SAₚₘₒₗₑ=HISₚₘₒₗₑ

16U1ₚₘₒₗₑ+30GFP-SAₚₘₒₗₑ=GLXₚₘₒₗₑ

11U1ₚₘₒₗₑ+11GFP-SAₚₘₒₗₑ=ARGₚₘₒₗₑ Equation 1

: : :

In the present case there are only two unknowns, the pmoles of LSB 1295.4 CP and the pmoles of GFP-SA, so at the minimum, only two of the equations listed above are required to define the complex. To determine the appropriate equations to employ, three independent amino acid analyses were performed for both the LSB 1295.4 virus and the GFP-SA, to compare the experimental compositions obtained to the known composition of each protein. Representative chromatograms for LSB 1295.4 virus and GFP-SA are shown in FIG. 4. A subset of the amino acids for which the known and experimental compositions agreed within +/−3% were then employed; the pmoles of GFP-SA was estimated from the pmoles of histidine in the complex, since this amino acid was unique to the fusion (FIG. 4B), and this value was then employed in the equations for arginine, leucine and asparagine/aspartic acid, to obtain an average for the pmoles of LSB 1295.4 CP present. The composition of the complex used as immunogen in the animal studies was determined to be an essentially equimolar ratio of GFP-SA to LSB 1295.4 CP (1.05:1.0). This translated into 559 pmoles of fusion tetramer per pmole of intact virion. If we assume a 1:1 ratio between tetramer fusion and biotinylated coat protein, a loading of 26% was achieved. This corresponding to over 2200 GFP molecules displayed per virion. As an internal control, the total pmoles of protein estimated by this composition-dependent procedure was employed to back-calculate protein concentration and this value compared to the concentration obtained using the data for all amino acids in a composition independent calculation, based solely on the molecular weight of the individual amino acids. The two concentrations determined were consistently within 10% of one another.

### EXAMPLE 24

### Preliminary Evaluation of the Immunological Response to GFP Decorated Virions

We compared the humoral immune response generated by GFP decorated virions with that obtained by immunization with the GFP-SA alone, in both mice and guinea pigs. For the preliminary study of induction of humoral immune responses in mice, antigen dose was normalized to 1 μg or 10 μg of GFP and administered without adjuvant. In the case of the guinea pig study, only the 10 μg GFP dose was considered and for a subset of animals, the antigen was co-administered with the adjuvant RIBI (Corixa, Hamilton, Mont.). As detailed in Table 2, an additional control consisted of GFP-SA combined with unbiotinylated LSB 1295.4 virus, in proportions mirroring the complex composition. This group was included to differentiate between the affect of physical association with the TMV capsid and the presence of the capsid alone on the immune response.

After one immunization, we observed augmented GFP specific antibody titers in a subset of mice from the groups administered complex, relative to the GFP-streptavidin alone or the streptavidin fusion mixed with the LSB 1295.4 virus, at both the 1 □g and 10 ug GFP doses (FIG. 5A). Following the second 1 μg GFP immunization, the mice administered the complex showed a robust immune response with endpoint titers that were significantly higher (P value=0.0001) than those observed within the groups administered the free GFP-SA in the presence or absence of unbiotinylated virus (FIG. 2B). At the 10 μg GFP dose, the differentiation between groups was not as marked (data not shown). Conjugation of GFP to the TMV capsid also elicited an improved immune response in guinea pigs. After the second immunization, GFP-specific antibodies were present in the sera of both animals administered the complex, while none were detectable in animals administered the mixture of GFP-SA and unbiotinylated virus (FIG. 5C). When the antigens were adjuvanted with RIBI, the differentiation between the groups was maintained with the mean antibody titers elicited by the complex being 5 to 20-fold higher than those observed with the free GFP-SA.

### EXAMPLE 25

The research reported here represents the results of the first year of a fruitful collaboration between LSBC and the group of Dr. Bennett Jenson and Dr. Shin-je Ghim at the James Graham Brown Cancer Center (JGBCC) at the University of Louisville. The overall topic of the collaboration is the development of new strategies for active and passive immunization against papillomavirus infection.

The first goal of the program was to develop a facile method for measuring papillomavirus neutralization in vitro, a necessary pre-requisite for our anti-papillomavirus drug development program. Since no method for measuring neutralization of the canine oral papillomavirus (COPV) in vitro existed at the start of this program, we licensed a neutralization assay system recently developed at the National Cancer Center, and developed the necessary COPV reagents. The COPV pseudovirions are now available for use in validating various COPV entry inhibitors, ranging from antibodies through small molecules.

We developed a novel papillomavirus vaccine composition for evaluation in vivo in the COPV-dog model. This vaccine is based on a fusion of a domain of the COPV L2 protein with the core-domain of the streptavidin protein. The fusion protein was expressed in Nicotiana plants and purified by affinity chromatography. The L2 vaccine fusion protein was loaded onto biotinylated tobacco mosaic virus, which LSBC has found previously to function as a powerful vaccine adjuvant. We delivered the plant-produced papillomavirus vaccines to our Brown Cancer Center collaborators on July 20^{th}, for their use in a dog vaccination and COPV challenge study. Given positive results from the dog trial, we hope to test this vaccine concept in human clinical trials in collaboration with the Brown Cancer Center at the University of Louisville.

Work on validation of a model for passive immunization against papillomavirus infection initiated on receipt of a collection of monoclonal antibodies raised against HPV-16 virus-like particles and COPV virion. We were able to identify a single neutralizing antibody from each set. Unfortunately, we found that the HPV-16 neutralizing monoclonal antibody was an IgM molecule, and as might be anticipated for an IgM, apparently required multivalent display of the antibody binding domains to achieve effective neutralization. If this project is to go forward, our recommendation would be to develop a new set of monoclonal antibodies and identify IgG isotype neutralizing antibodies. The COPV-neutralizing monoclonal antibody that we identified was an IgG molecule. We have expressed both the COPV neutralizing IgG and the FAB domain of the antibody in plants, and have purified test quantities of the products. Due to some unanticipated technical difficulties, we have, however been unable to confirm the neutralization activity of the plant-produced molecule and the original IgG hybridoma-derived antibody, but work will continue with the goal to provide the experimental data to support a passive immunization trial in dogs, as the neutralization activities of the antibodies are confirmed.

Our progress towards achieving each of the goals of the collaborative program is summarized in the Research Report, below. The financial report details the number of hours LSBC personnel dedicated to the research project, as well as total costs spent on achieving the aims of the collaborative program.

### Part 1: Build and Validate Copv Pseudovirions.

Since no facile in vitro neutralization assay for COPV exists, we decided to build COPV Pseudovirions (PsV) for use in a neutralization assay developed at the National Cancer Institute, and licensed by LSBC (Pastrana et al., 2004). We obtained reagents for developing the PsV from the NCI, and used these in the development of COPV PsV. FIG. 1 shows that the COPV PsV we built are indeed able to transduce Human embryonic kidney 293 cells, and shows the that a 1:800 dilution of PsV stock was the appropriate amount to add to 293 cells to achieve a relative light unit reading of approximately 10,000, which is optimal for measurement of virus neutralization when the threshold for neutralization is set at a 50% reduction of the maximum RLU reading. This concentration of PsV was used in subsequent assays.

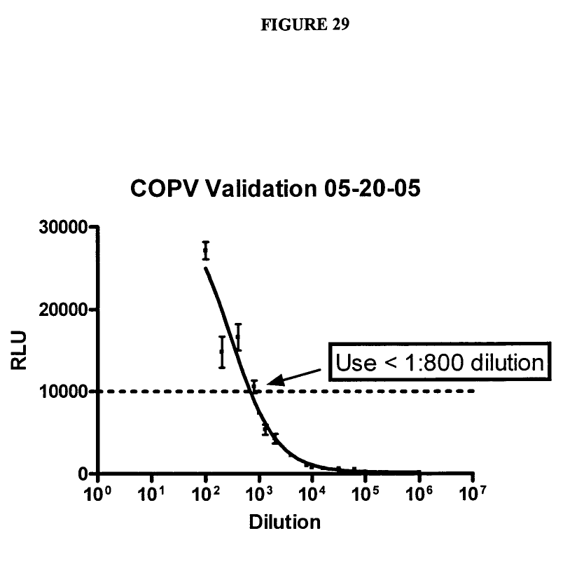

FIG. 29. shows Validation of COPV PsV Assembly and Transduction of HEK 293 Cells. Various dilutions of COPV PsV stock were added to HEK 293 cells as described (Buck et al., 2004; Pastrana et al., 2004), and secreted alkaline phosphatase activity in supernatant media assayed after three day incubation. A 1:800 dilution of COPV PsV induced an appropriate amount of SEAP expression for use in a virus neutralization assay.

Part 2: Assay for Neutralization of COPV PsV with COPV Sera and Monoclonal Antibodies

We validated the utility of the COPV PsV using neutralizing monoclonal antibodies from Dr. Ghim (FIG. 2). This shows that COPV monoclonal antibody 7-2, raised against COPV Virion, could neutralize COPV PsV infectivity, but COPV mAb 14-1 could not.

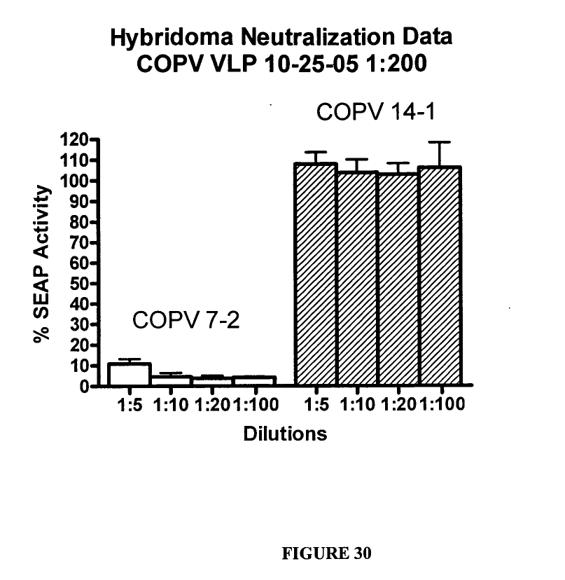

FIG. 30. shows neutralization of COPV PsV by COPV mAb 7-2, but not mAb 14-1. The percentage reduction in SEAP activity in wells of cells incubated with COPV mAb is indicated. Assays were repeated in triplicate, and the error bars reflect the between-well variation for triplicate assays.

Unfortunately, we have subsequently experienced some difficulties in the use of the COPV PsV neutralization assay and were unable to confirm that a polyclonal antiserum sent by Dr. Ghim would neutralize the COPV PsV. These data are surprising, and we will require further repetition to confirm the result.

Part 3: Clone and Express in Plants cDNAs that Encodes a Papillomavirus Neutralizing Antibody FAB Domain

### COPV

Nine COPV mouse hybridoma lines (C1 16-3, C1 25-3, C1 28-3, C1 6-3, C1 7-2, C1 14-1, C1 15-3, C1 17-2, C1 22-1) were evaluated using the in vitro neutralization assay against COPV PSV as described previously. Line C 17-2 was the only line of the nine that contained neutralizing activity, as determined by a 50% reduction in infectivity compared to control at any tested dilution. (Table 1) Dilutions tested ranged from 1:10 to 1:10,000 of hybridoma supernatant.

TABLE 1
Mouse hybridoma line isotype and viral neutralization.

| COPV | Isotype- | Isotype- | COPV |
|---|---|---|---|
| Hybridoma | Heavy Chain | Light Chain | Neutralization |
| C1 16-3 | IgG2b | Kappa | — |
| C1 25-3 | IgG2b | Kappa | — |
| C1 28-3 | IgG2b | Kappa | — |
| C1 6-3 | nd | nd | — |
| C1 7-2 | IgG1 | Kappa | +(1:10,000) |
| C1 14-1 | nd | nd | — |
| C1 15-3 | nd | nd | — |
| C1 17-2 | nd | nd | — |
| C1 22-1 | nd | nd | — |
| HPV-16 | | | HPV-16 |
| Hybridoma | | | Neutralization |
| 16 A5 | IgG2a | Kappa | — |
| 16 C9 | IgA | Kappa | — |
| 16 E9 5 | IgA | Kappa | — |
| 16 F2 | IgG2b | Kappa | — |
| 16 G4 | IgM | Kappa | +(1:10,000) |

Hybridoma C1 7-2 was determined to be gamma 1/kappa isotype by PCR and ELISA based experiments are described in the quarterly report. The heavy and light chain genes were amplified by PCR and assembled into a mAb and a FAB expression ORF and cloned into plant viral expression vectors for evaluation. Systemic, viral infected plant tissue was harvested and the secreted protein fraction isolated. Plant extracts were evaluated for COPV mAb and FAB production by Coomassie stained SDS-PAGE under both reducing and non-reducing conditions. The accumulation of the desired mAb product was seen as a novel protein in the extract at the expected size of approximately 150 kDa and the FAB was identified as a novel protein migrating at the expected size of 45 kDa.

### HPV-16

The HPV-16 neutralization activities of antibodies from the five mouse hybridoma lines, 16A5, 16C9, 16E9 5, 16F2 and 16G4 were evaluated using the in vitro neutralization assay. Line 16G4 was the only line tested that demonstrated neutralization activity as determined by a 50% reduction in infectivity compared to control at any tested dilution. (Table 1) Dilutions tested ranged from 1:10 to 1:10,000 of hybridoma supernatant.

Hybridoma 16G4 was determined to be mu/kappa isotype by PCR and ELISA based experiments. To enable plant expression of the mnAb and FAB proteins, the heavy chain isotype was switched to mouse gamma 2a, chosen as it is a TH1 induced isotype. The heavy and light chain genes were PCR amplified, assembled into a mAb and a FAB expression ORF and cloned into plant viral expression vectors for evaluation. Systemic, viral infected plant tissue was harvested and the secreted protein fractions isolated. Plant extracts were evaluated for HPV mAb and FAB production by Coomassie stained SDS-PAGE under both reducing and non-reducing conditions. The accumulation of the desired mAb product was seen a novel protein in the extract at the expected size of approximately 150 kDa and the FAB was identified as a novel protein migrating at the expected size of 45 kDa. The accumulation of the mAb and FAB proteins were further verified by reactivity with anti-mouse gamma and kappa antibodies on western blots. Additionally, the binding activity of the plant produced mAb and FAB proteins were verified by their ability to bind HPV-16 PSV coated ELISA plates.

### Part 4: Assay for Recombinant Plant-Produced Antibody Virus Neutralization Activity

Preliminary virus neutralization experiments using plant extracts indicated plant impurities were not compatible with the assay, and therefore, purification of the expressed proteins was necessary to obtain reliable assay results.

### COPV

### Purification of COPV Antibodies

Purification of the COPV 7-2 IgG mAb from hybridoma culture supernatant and infected plant tissue was performed by affinity chromatography with Protein G Sepharose that selectively binds the constant regions of most antibodies. This approach was not useful for purification of the FAB molecules, as the majority of the constant region is absent. A biochemical strategy using ion-exchange chromatography and hydrophobic interaction chromatography (HIC) was developed for purification of FAB proteins from plant extracts. The resulting process was designed to be compatible with increased biomass and purity requirements for production of material for subsequent animal experiments. The purified hybridoma and plant COPV mAb and FAB proteins were analyzed by Coomassie stained SDS-PAGE under non-reducing conditions. Gel analysis of the purified mAbs shows the expected major band of the full-length approximately 150 kDa product. The plant produced mAb, and to a lesser extent, the hybridoma purified mAb, contain minor impurities which have been previously shown to be antibody fragments resulting from proteolysis during host expression, and contain sufficient constant region to bind Protein G. The purified COPV FAB migrates at the expected size of 45 kDa and contains low levels of uncharacterized impurities.

### Neutralization Activity

The COPV neutralization assay has proven problematic and this issue is currently being resolved. The initial COPV hybridoma screening assay identified line 7-2 as neutralizing, but subsequent assays have been unreliable and these results have not been repeated. In addition, we have not obtained neutralization results for the purified mAb and FAB proteins.

Determination of neutralizing activity of the COPV FAB as compared to the cognate mAb will determine the required dosing for the planned animal experiment. If the neutralizing activity of the FAB is significantly different than the COPV mAb, the design of the animal experiment will be re-evaluated.

### HPV-16 Purification of HPV-16 Antibodies

Purification of the HPV-16 G4 IgG mAb from plant tissue was performed by affinity chromatography using Protein G Sepharose. A biochemical strategy using ion-exchange chromatography was developed which allowed sufficient purification for the neutralization assay. The purified plant HPV mAb and FAB proteins were analyzed by Coomassie stained SDS-PAGE under non-reducing conditions. (FIG. 3). Gel analysis of the purified mAb shows the major band of the full-length 150 kDa product and minor proteolytic impurities. Gel analysis of the purified FAB protein shows the protein is a doublet at the expected size of 45 kDa. The presence of a doublet was not anticipated as there are no N-linked glycosylation sites in the predicted protein sequence. The presence of a doublet is likely due to incomplete cleavage of the propeptide linking sequence located between the heavy and light chains. The presence of this extra sequence would not be expected to adversely influence the binding ability of the FAB as antibody heavy and light chains have been linked in scFv's and retained full-binding activity. The HPV-16 FAB was also expressed as a molecular dimer or FAB′, generated by the addition of a leucine-zipper domain which promotes dimerization of the FAB. The purified FAB′ produced an expected 90 kDa band when analyzed by non-reducing SDS-PAGE indicating the protein is dimeric.

### Neutralization Activity

HPV-16 G4 positive control hybridoma supernatant and purified plant antibodies were evaluated for neutralization of the HPV-16 PSV. (Table 2) Serial dilutions of the samples were tested and the EC50 was determined as the concentration at which resulted in a 50% reduction in infectivity as compared to control. The hybridoma derived mAb, plant produce mAb and FAB′ all have positive neutralizing activity while the FAB did not neutralize at any tested concentration (0.25-250 μg/ml).

TABLE 2
Virus neutralization with HPV-16 G4 antibodies.

| HPV-16 G4 | | Neutralization | Antibody |
|---|---|---|---|
| Antibody | Isotype | EC50 [c] | valency |
| HPV mAb | IgM | 0.01 μg/ml | Decamer |
| hybridoma | | | |
| HPV mAb plant | IgG2a | 1 μg/ml | Dimer |
| HPV FAB plant | IgG2a Fd | negative | Monomer |
| HPV FAB′ plant | IgG2a Fd | 20 μg/ml | Dimer |

The neutralizing activity of the HPV-16 G4 antibody appears to increase as the number of binding regions or ‘valency’ increases. These results indicate that G4 may be a low affinity antibody, which is not uncommon for IgM, and appears to require multiple binding regions resulting in a higher avidity sufficient for efficient neutralization. Based on these observations, it is our recommendation that the HPV-16 G4 antibody is not suitable for further evaluation.

### Part 5: Express COPV L2 Fragments in Tobacco Using Recombinant TMV Technology

### Introduction:

Previous work at LSBC had shown that rabbit and human papillomavirus peptides are highly immunogenic when displayed on the surface of TMV, and induce virus neutralizing antibodies, and protective immunity in rabbits (unpublished data). We have been interested in exploiting the adjuvant property that the TMV structure offers, by finding a way to display whole proteins or protein domains on the surface of TMV. In a separate project, we found that we could biotinylate recombinant TMV particles that had been engineered to display a reactive lysine on the surface of the virus particle. When fusion proteins between the green fluorescent protein and the streptavidin core protein were displayed on the surface of TMV and used as vaccines in mice and guinea pigs, we found that the TMV:SA-GFP fusion proteins were significantly more immunogenic than unconjugated control proteins. We found that vaccinated animals produced both higher titer vaccine-specific antibodies and larger numbers of peptide-specific CD8+ T-cells in response to vaccination with the TMV-strepravidin GFP complex. We therefore decided that the best approach to developing a plant-produced L2 vaccine would be to express the COPV L2 peptide of interest as a fusion protein with the core of streptavidin, and to conjugate this to TMV as we had done previously with the SA-GFP protein. The following narrative describes the experimental approach and results.

### I. Construction of the Recombinant COPV L2 Subunit Fused to Streptavidin

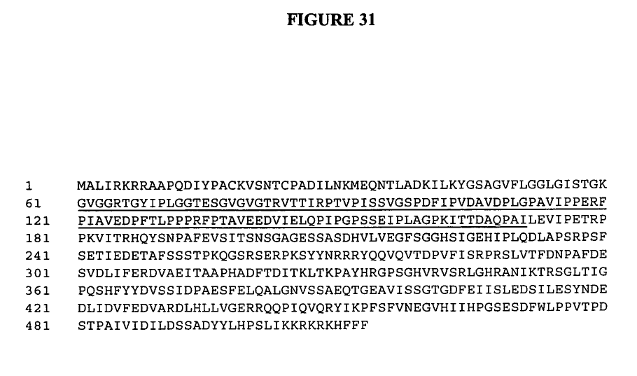

A TMV GENEWARE® vector was constructed to express in Nicotiana plants a fusion protein between the 112 amino acid domain corresponding to amino acids 61-171 of COPV L2 protein (FIG. 31), fused to the streptavidin core protein. The resulting construct, pLSB1825, contained the recombinant COPV-L2 region fused to the N-terminus of the streptavidin (L2:SA). The plasmid pLSB 1825 was confirmed by sequencing and its nucleotide and the deduced amino acid sequences for the L2:SA fusion protein are shown in Appendix 1, sequence COPV L2-SA. FIG. 31. The amino acid sequence of COPV L2 Protein (Genbank accession # NP_{—}056818). Underlined sequence indicates the region selected to produce the recombinant L2:SA fusion.

II. Screening for Expression of pLSB1825 (L2:SA) in Nicotiana benthamiana Plants

To evaluate the expression of the pLSB 1825 construct, RNA transcripts were generated using the mMESSAGE mMACHINE T7 transcription kit (Ambion, Austin, Tex.) and subsequently used to inoculate 22 day old N. benthamiana plants. Following inoculation, the typical mosaic phenotype was observed on the systemically infected tissue. The expression of L2:SA (PLSB1825) was evaluated, seven days post inoculation, by extracting proteins from a small piece of leaf tissue. The tissue was homogenized in either TRIS (100 mM TRIS-HCl, pH 8.0) or 1×SDS Loading Buffer (78 mM TRIS (pH 7.0), 10% SDS, 0.05% bromophenyl blue, 6.25% glycerol, 10% β-mercaptoethanol). The crude extracts in TRIS buffer were clarified by centrifugation at 15,000×g for 3 minutes. The supernatants were collected and diluted in 4×SDS Loading Buffer (3:1; extract:4×SDS). Both samples, TRIS and 1×SDS Loading Buffer, were boiled at ˜100° C. for 5-10 minutes, and the 1×SDS Loading Buffer sample was clarified as described above. Crude extracts, isolated from uninoculated and pLSB 1825 inoculated plants, were separated on an SDS-PAGE and stained with Coomassie. There was a ˜30 kDa protein band unique to both L2:SA samples (lanes 5 and 6) and absent in the uninoculated negative controls (lanes 2 and 3). To confirm the identity of the putative L2:SA band, a Western blot was performed on the same set of crude extracts. Rabbit anti-streptavidin serum (Sigma, St Louis, Mich.) and goat anti rabbit IgG (H+L)-HRP (Biorad, Hercules, Calif.) were used as primary (at 1:50,000 dilution) and secondary (at 1:10,000 dilution) antibodies, respectively. The ECL Plus Detection kit (Amersham Biosciences, Piscataway, N.J.), was employed for detection. Both L2:SA extracts showed strong reactive bands (lanes 3 and 4) near the 30 kDa marker, and no signal was present in the negative control samples (lanes 1 and 2). The 30 kDa band is close to the predicted molecular weight of the fusion (25 kDa). The observed size difference may reflect some post-translational modifications occurring in planta or the conformation of the fusion may retard electrophoretic mobility. In addition to the 30 kDa band, there are other smaller minor bands that may represent multiple truncations of the full L2:SA fusion. These preliminary results suggest successful expression of the L2:SA fusion in N. benthamiana and demonstrate specific detection of the streptavidin portion of the fusion.

### COPV L2 Streptavidin (SA) Extraction and Purification

The initial isolation of the L2:SA fusion from pLSB 1825 infected tissue was based on a protocol developed for a green fluorescent protein (GFP)-streptavidin fusion. This procedure is summarized in flow diagram format in FIG. 6A. Infected N. benthamiana tissue was homogenized with a three-fold excess of 0.1M NaPO₄ pH7.5 buffer containing 0.01% (w/v) sodium metabisulfite. To the clarified supernatant, ammonium sulfate was added to 25% of saturation, and then a subsequent 50% ammonium sulfate precipitation was performed. The differential precipitation was intended to separate the TMV from the COPV L2 SA and subsequently concentrate the streptavidin fusion prior to chromatography. The 50% ammonium sulfate pellet was resuspended in phosphate buffer, dialyzed, and affinity purification was performed on an AKTA purifier 100 (Amersham Biosciences) using a 2 ml iminobiotin resin column (Pierce, Rockford, Ill.).

SDS-PAGE analysis (FIG. 6B) showed the purified product migrated as a doublet at ˜30 kDa, (F20-F22) in agreement with the preliminary screening results (FIG. 5). However, prominent putative breakdown species migrating at ˜14 kDa and comprising approximately 60% of the total protein were also present. Full length mass spectrometry and tryptic MALDI confirmed the identity of the full length and truncation species. The yield of full-length product was estimated at 8 mg/kg infected tissue. Western blot analysis was also performed which clearly demonstrated that the 30 kDa and 14 kDa doublet in the elution fractions were anti-SA reactive. Of note was the presence of a prominent anti-SA reactive band in the 25% ammonium sulfate pellet, which was discarded during processing. This suggested that the precipitation profile of COPV L2 SA differed from that of GFP-SA and optimization of the processing was required.

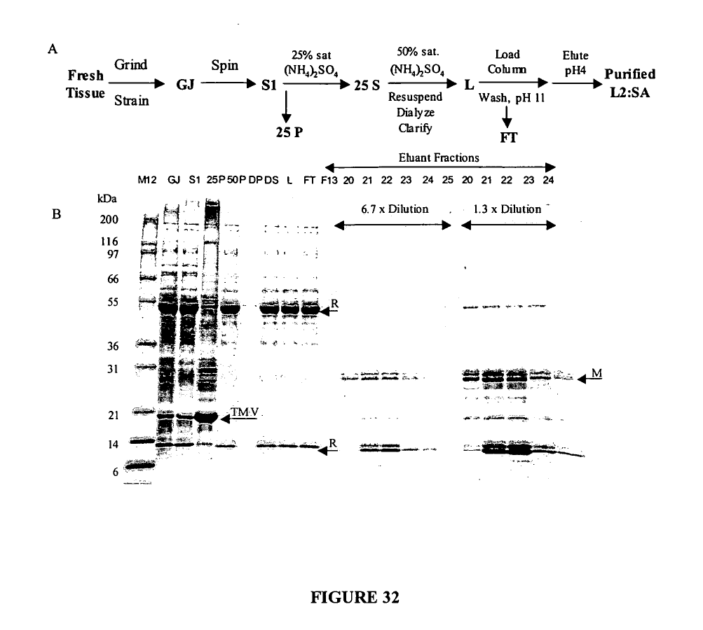

FIG. 32. A. Process flow diagram for the principal steps in COPV L2-SA purification, from infected plant tissue through affinity chromatography. B. SDS-PAGE analysis for initial COPV L2-SA purification. Legend; GJ, homogenized plant extract “Green Juice”; S1, initial supernatant; 25P, 25% saturated ammonium sulfate pellet; 25S, 25% saturated ammonium sulfate supernatant; L, load (dialyzed 50% ammonium sulfate fraction); FT, flow through; M12, Invitrogen Mark 12 protein marker; 50P=50% ammonium sulfate pellet; DP, precipitate from 50% ammonium sulfate fraction dialysis; DS=supernatant from 50% ammonium sulfate fraction dialysis; F## eluant fractions; M, monomer form of COPV L2-SA; R, rubisco large and small subunits; TMV, TMV coat protein.

Thus, for the next trial extraction, the 25% ammonium sulfate pellet was carried forward for affinity purification, since it appeared to contain the majority of the intact streptavidin fusion. Also, extraction was performed in a low ionic strength buffer (25 mM Tris maleic acid, pH 7.5) and the competitive cation polyethyleneimine (PEI) added to the initial green juice at 0.4% w/v. PEI was incorporated into the procedure to improve the clarity of the initial supernatant. The modified procedure is outlined in FIG. 8A. With PEI addition, the green pigment previously associated with the supernatant (FIG. 8B, lane S1) partitioned into the pellet (FIG. 8B, lane P1), as did the rubisco and the majority of the TMV. As a result the 25% ammonium sulfate precipitation yielded substantially purified COPV L2-SA (lane 25P), since the majority of the host proteins remained soluble (lane 25S). Affinity purification of the COPV L2-SA from the resuspended 25% ammonium sulfate pellet yielded a 30 kDa doublet, and no degradation products were observed. This indicated that the prominent truncation species observed in FIG. 6 were due to concentration of the low levels present in the 25% supernatant and not due to in vitro degradation. Analysis of protein mass by Matrix Assisted Laser Desorption-Ionization Time of Flight mass spectrometry (MALDI-TOF) (FIG. 9) indicated that molecular weight of the major species was 24,897 Da, matching the expected species with N-terminal methionine cleavage. In-gel tryptic digests were also performed on both the principal band and the minor upper band. Both bands matched COPV L2-SA, with near complete coverage of the expected peptide map obtained. For the upper band a +79 Da peak was present for one of the fragments, suggesting possible phosphorylation [+80] of the oxygen of Ser, Thr or Asp, a modification to L2 proteins that has been reported in the literature (Xi S Z, Banks L M. J Gen Virol. 1991; 72:2981-8). In addition, recoveries were approximately 10-fold higher than the initial extraction, at 90 mg/kg, as determined by BCA analysis.

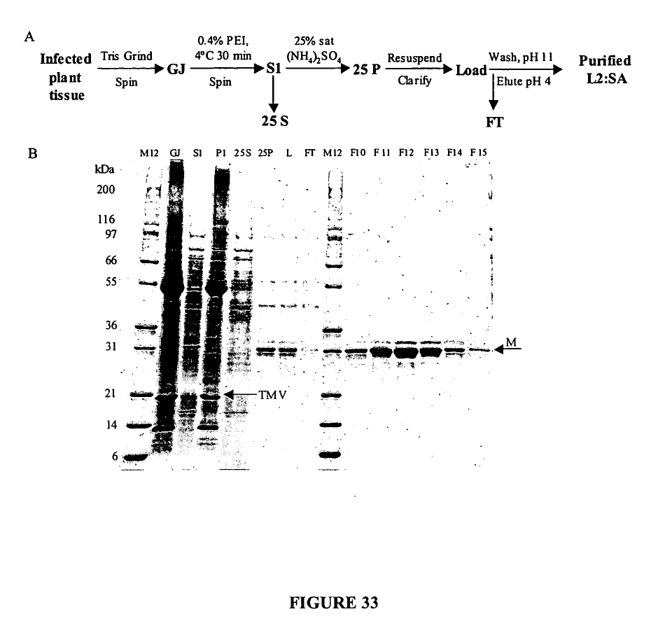

FIG. 33. shows a Optimized process flow diagram for the principal steps in COPV L2-SA purification, from infected plant tissue through affinity chromatography. B. SDS-PAGE analysis for the optimized COPV L2-SA purification process. Legend; GJ, homogenized plant extract “Green Juice”; S1, initial supernatant; 25P, 25% saturated ammonium sulfate pellet; 25S, 25% saturated ammonium sulfate supernatant; L, load (25% ammonium sulfate fraction); FT, flow through; M12, Invitrogen Mark 12 protein marker; F## eluant fractions; M, monomer form of COPV L2-SA; TMV, TMV coat protein.

With further minor modifications noted below, the optimized protocol (FIG. 33) yielded sufficient quantities of full-length protein with greater than 90% purity, permitting vaccine production to proceed. Two manufacturing runs were performed to obtain the COPV L2-SA fusion. The amount of ammonium sulfate used to precipitate L2-SA was increased from 25% to 30% of saturation to ensure maximal recovery of the target protein. To minimize endotoxin contamination, all glassware was baked and the buffers, prepared using water for irrigation (WFI) and dedicated reagents, were 0.2 um sterile filtered. In order to concentrate the affinity purified product for use in the vaccines, the pooled peak eluent fractions were precipitated with 50% ammonium sulfate. Subsequent dialysis yielded highly purified COPV L2-SA with recoveries of 70-75% for the concentration step. From a total of 600 grams of infected plant material approximately 36 mg of final product was obtained, as determined by AAA, corresponding to an overall yield of 60 mg/kg.

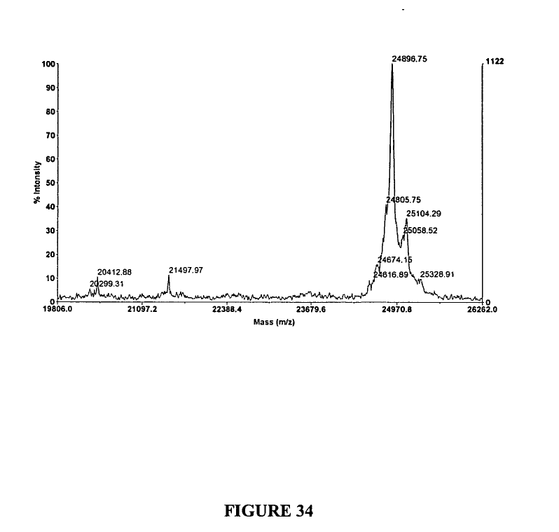

FIG. 34. Molecular weight mass spectrometry for affinity purified COPV L2-SA, prior to BEI treatment. Full length COPV L2-SA (aa 1-242) has an expected molecular weight of 25025.97 Da (M+H). With methionine cleaved (aa 2-242) the expected molecular weight is 24894.77 Da (M+H), which matches the major peak observed (24896.75 Da) within the 0.05% confidence interval. No acetylation of the N-terminal glycine was detected.

### Formation of the COPV L2-SA TMV Complex and Vaccine Preparation

The loading of the COPV L2 streptavidin fusion onto biotinylated TMY was characterized principally by SDS-PAGE band shift analysis. FIG. 10 A illustrates schematically the different forms in which streptavidin can exist, as a function of temperature and the presence of biotin. For streptavidin alone, the tetrameric form is stable up to 60° C. in the presence of SDS, with disassociation to the monomer occurring at higher temperatures. However, when biotin is present in molar excess, i.e. sufficient quantities to occupy the four biotin-binding sites of the tetramer, stability is increased, with minimal monomer disassociation occurring with heating to 95° C. The different streptavidin forms can be visualized by protein gel electrophoresis (FIG. 10 B). In the absence of biotin the 100 kDa COPV L2 SA tetramer (migrating at 116 kDa) shifts to the 25 kDa monomer with increasing temperature, with tetramer stabilization by biotin evidenced by the notable reduction in monomer level at 95° C. FIG. 10 B also shows that with unbiotinylated LSB 1295.4 virus present (coat protein migrating at ˜20 kDa) the tetramer to monomer transition for COPV L2 SA is unaffected.

For complex loading, the COPV L2 streptavidin and the biotinylated LSB 1295.4 virions were combined in a 1:1 molar ratio and incubated for 4 hours at room temperature, with mild agitation, to permit streptavidin fusion loading of the capsid to occur. These conditions translate into a maximum possible loading of 25%, or approximately 536 tetramers (2144 COPV L2 fragments) per capsid. Typically, following the four hour incubation, the complex preparation is precipitated by polyethyleneglycol (PEG), to remove soluble free tetramer and confirm tetramer/biotinylated capsid association. However, unlike previous streptavidin fusions, e.g. GFP-SA, the free COPV L2-SA tetramer was precipitated by all concentrations of PEG tested (1%-4% w/v). Protein gel electrophoresis of the COPV L2-SA, biotinylated TMV mixture, with heating to 60° C., does not confirm complex formation. Prior testing had demonstrated that if biotinylated TMV virions and COPV L2 SA were separately combined with SDS-PAGE loading buffer and then mixed, tetramer/biotinylated coat protein association still occurs. The detergent SDS causes the TMV capsid to disassociate, even in the absence of heating, so if a band shift corresponding to tetramer/biotinylated coat protein is detected, it is not possible to attribute this to complex formation prior to sample preparation for SDS-PAGE analysis. This issue was resolved by addition of a 5-fold excess of biotin, which saturates any unoccupied biotin binding sites, thereby preventing any additional association from occurring after detergent addition.

The SDS-PAGE analysis for the two COPV L2-based antigens and the TMV 1295.4 control antigen is shown in FIG. 10 C. All three antigens were treated with 5 mM binary ethylenimine (BEI) at 37° C. for 48 hours to inactivate the TMV and for sterilization. Excess BEI was neutralized by the addition of a 3-fold molar excess of sodium thiosulfate, the pH adjusted and the antigens diluted to their target concentrations (1.7 mg/ml for COPV L2-SA and 1.2 mg/ml for TMV) before vialing. Samples of each antigen were combined with SDS-PAGE loading buffer, aliquots heated to 60° C., 95° C. or left at room temperature and assessed by polyacrylamide gel electrophoresis. As expected, the TMV coat protein migrated as a single 20 kDa band at all temperature treatments and was unaffected by the BEI treatment (compare to FIG. 10A). For the COPV L2 SA, the tetramer to monomer transition with heating from 60° C. to 95° C. was evident in the absence of biotin, as was the tetramer stabilization at 95° C. when biotin was added. However, a monomer band was detected in all lanes independent of temperature, which was absent for the non-BEI treated streptavidin fusion (see FIG. 10A). This result suggests modification of the COPV L2-SA by BEI, generating a subset of the fusion that was incapable of self-association. For the complex, the TMV coat protein band was in the form of a doublet, the upper band representing the biotinylated coat protein, with the level of biotin addition estimated at greater than 80%. Bands migrating above the streptavidin fusion tetramer, corresponding to biotinylated coat protein association, were present for both the room temperature and 60° C. samples in the absence of biotin. These bands were also present in the room temperature sample for complex preincubated with biotin, confirming complex formation and indicating that BEI treatment did not affect the complex. Minimal free tetramer was detected, suggesting near complete loading of the fusion added. For the free biotin containing complex sample, the complex bands were reduced to tetramer and free coat protein with heating to 60° C., the result of reduced biotin/streptavidin affinity at the elevated temperature and exchange of the biotinylated coat protein with the free biotin pool, which was present at a 5-fold molar excess. At all temperatures a COPV L2 SA monomer band was also detected in the complex lanes, similar to the band observed in the BEI treated COPV L2-SA, although not as intense.

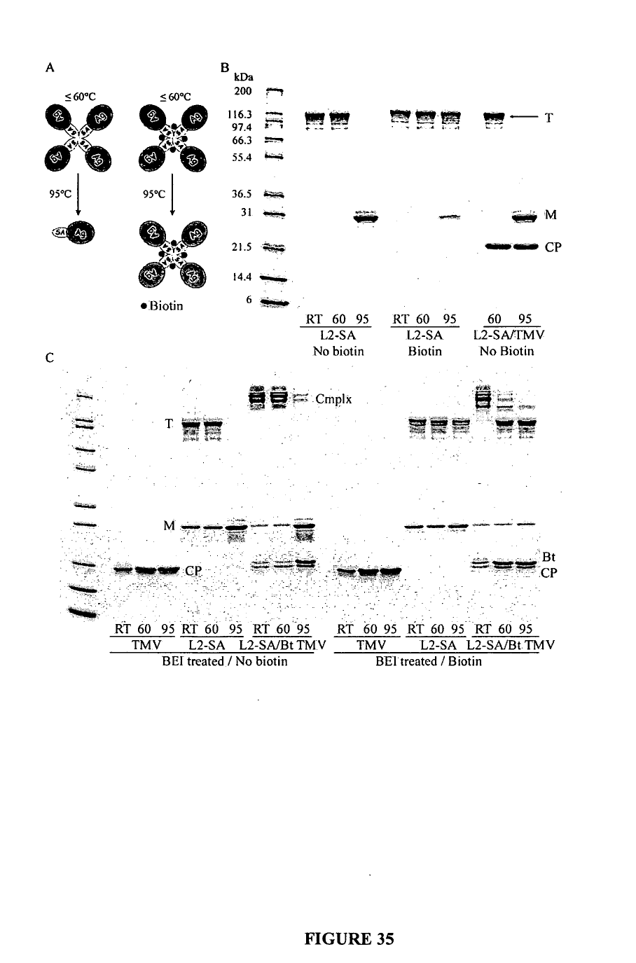

FIG. 35. Band shift analysis for the COPV L2 streptavidin fusion, alone and complexed to biotinylated 1295.4 TMV capsids. A. Schematic diagram of a streptavidin (SA) antigen (Ag) fusion and its quaternary structure as a function of temperature and the presence of biotin. B. SDS-PAGE migration pattern for the COPV L2-SA fusion (L2 SA) alone or mixed with unbiotinylated 1295.4 TMV (TMV). No samples were BEI treated and when biotin was added it was present at a 5-fold molar excess. C. SDS-PAGE migration pattern for 1295.4 TMV, COPV L2 SA and the COPV L2 SA, biotinylated TMV mixture (L2-SA/Bt TMV). All samples were BEI treated and when biotin was added it was present at a 5-fold molar excess. Legend; T, tetramer; M, monomer; CP, TMV coat protein; Cmplx, complex of COPV L2 SA tetramer and biotinylated coat protein; Bt CP, biotinylated coat protein.

The three vialed antigens were provided to Quality Control for full release testing. The antigen lot numbers, the release specifications and the results for each antigen are summarized in Table 3 below. The three antigens conformed to all release specifications and were shipped to the University of Louisville on July 20^{th}. The antigens were received in good condition and the dog immunization and COPV challenge study is anticipated to commence within the next 4-6 weeks. Sufficient quantities of each antigen were shipped to dose at 200 ug for the COPV L2 fragment, following the tentative study protocol outlined in Table 4.

TABLE 3
Release specification and product certification for the
manufactured COPV L2 and control antigens.

| | BEI Treated | BEI Treated | BEI Treated |
|---|---|---|---|
| | Certification No. | | |
| | No. 038 | No. 039 | No. 040 |
| | Lot No. | | |
| | 1295.423Jun05CP | 182523Jun05L2 | 182523Jun05L2CP |
| Concentration (AAA) | 1.13 mg/mL | 1.51 mg/mL | 2.94 mg/mL |
| Target | 1.2 mg/mL | 1.7 mg/mL | 2.9 mg/mL |
| Appearance | Opalescent, colorless, clear | Opalescent, colorless, clear | Opalescent, colorless, clear |
| Specification | No foreign particulates | No foreign particulates | No foreign particulates |
| Purity, by SDS-PAGE | 98.4% | 94.1% | 97.2% |
| Specification | >90% | >90% | >90% |
| pH | 7.6 | 7.61 | 8.26 |
| Specification | 7.5 +/− 1.0 | 7.5 +/− 1.0 | 7.5 +/− 1.0 |
| Endotoxins | <0.500 EU/mL | <0.500 EU/mL | 0.593 EU/mL |
| Maximum | 5 EU/mL | 5 EU/mL | 5 EU/mL |
| Bioburden Assay* | Passed | Passed | Passed |
| Local Lesion Assay** | Passed | Passed | Passed |
| Tryptic MALDI | Full Length Present | Full Length Present | Full Length Present |
| Storage Buffer | PBS | PBS | PBS |
| Storage Conditions | −20 C. | −20 C. | −20 C. |

*The sample passes this assay when no colonies are detected on growth media incubated at room temperature for 4 days, followed by 4 more days at 33 C.
**The sample passes this assay if an average of one or fewer lesions develops on three separate inoculated leaves of a local lesion host plant.

TABLE 4
Study Protocol Proposed for Evaluation of COPV L2 Vaccines in Dogs

| | | | | ug COPV | ug 1295.4 |
|---|---|---|---|---|---|
| | | | # | L2/ | TMV/ |
| Group | Vaccine | # animals | immunizations | dose | dose |
| 1 | COPV L2 | 4 | 3 | ~200 | 0 |
| 2 | COPV L2/ | 4 | 3 | ~200 | ~255 |
| | K-TMV | | | | |
| | complex | | | | |
| 3 | K-TMV | 2 | 3 | 0 | ~255 |

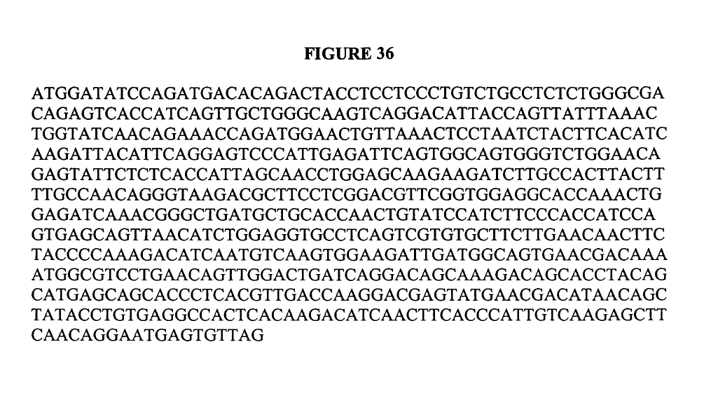

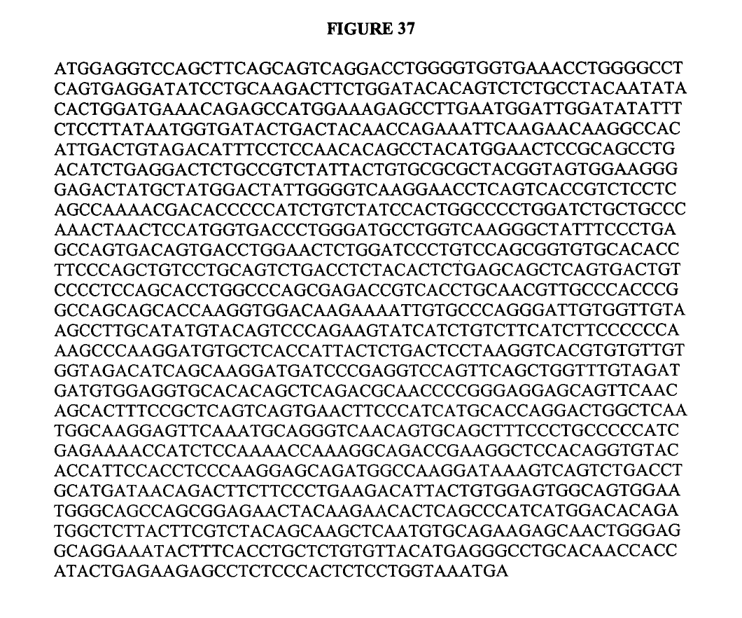

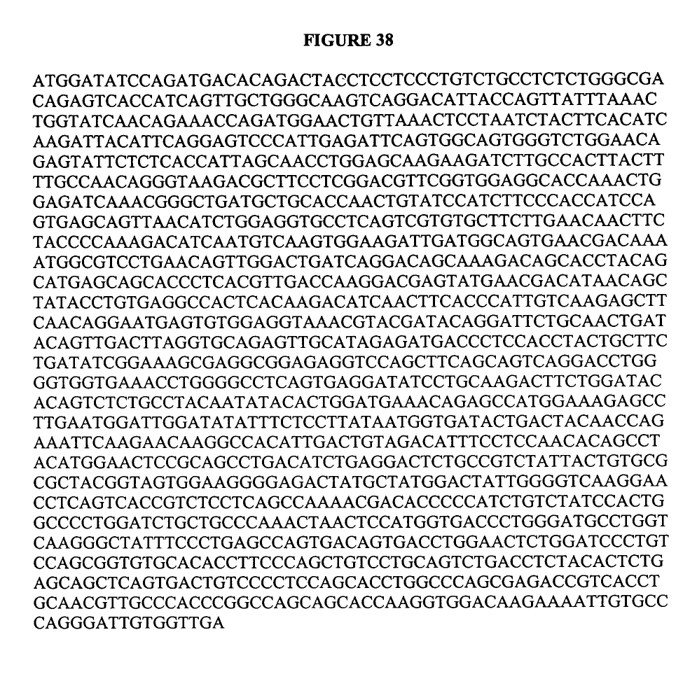

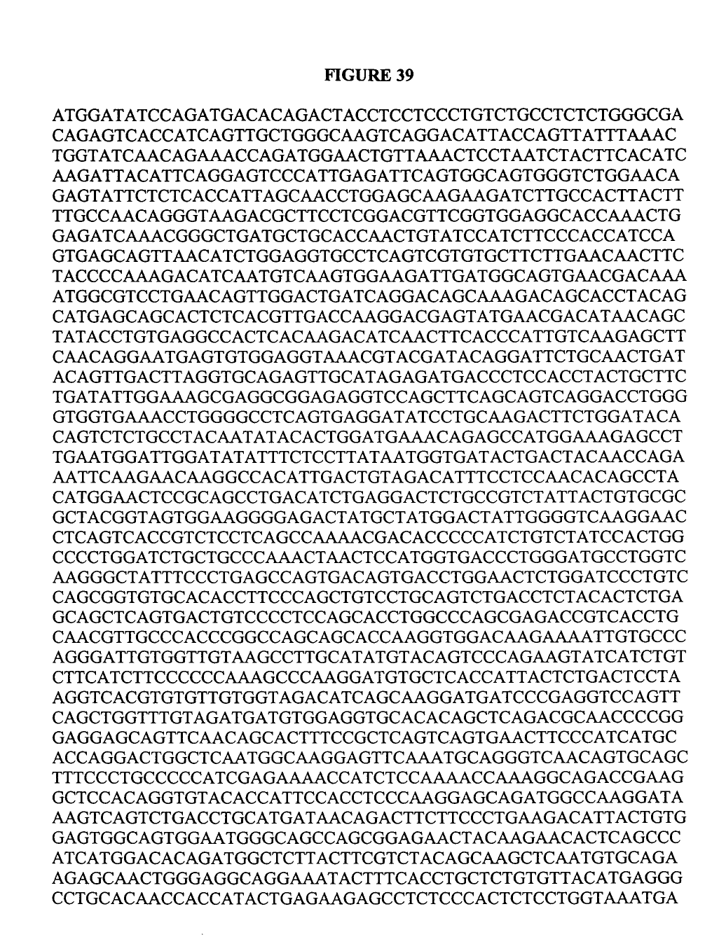

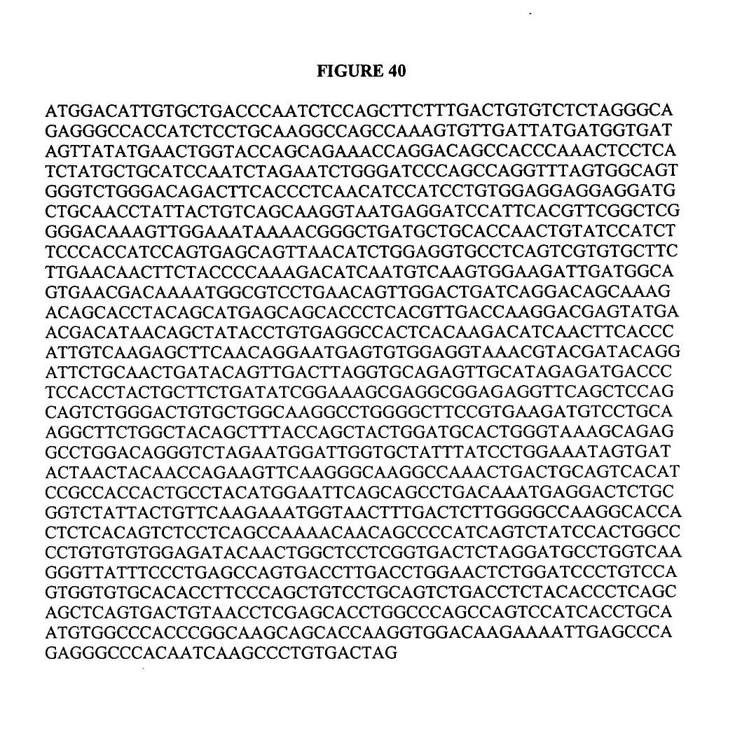

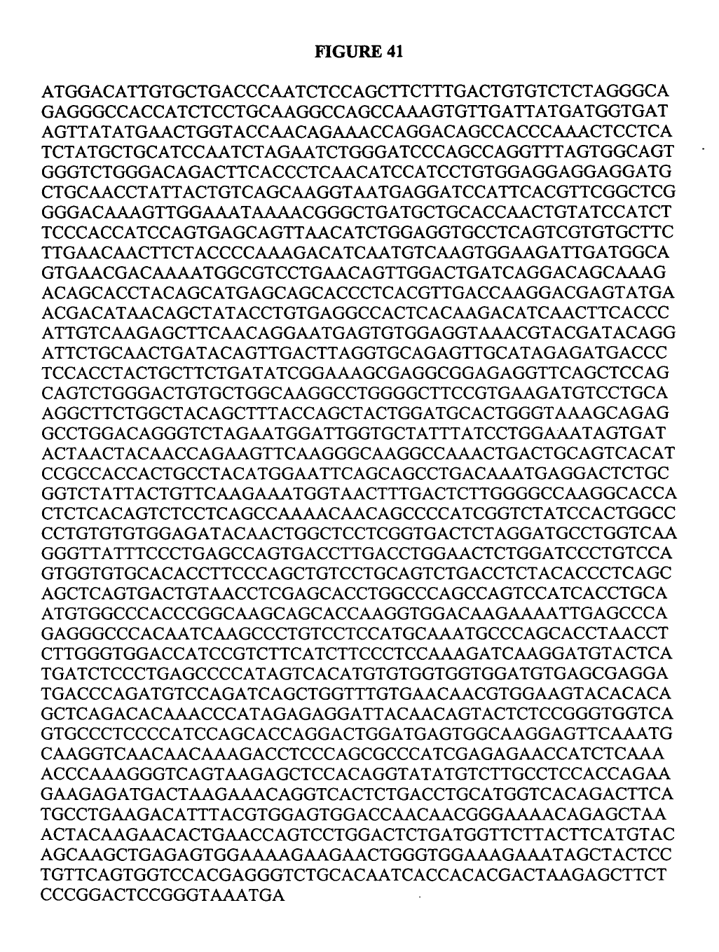

FIG. 36 shows the COPV C17-2 Light Chain. FIG. 37 shows the COPV C17-2 Heavy Chain. FIG. 38 shows the COPV C17-2 FAB nucleic acid sequence. FIG. 39 shows the COPV C17-2 mAb nucleic acid sequence. FIG. 40 shows the HPV-16 G4 FAB nucleic acid sequence. FIG. 41 shows the HPV-16 G4 mAb nucleic acid sequence.

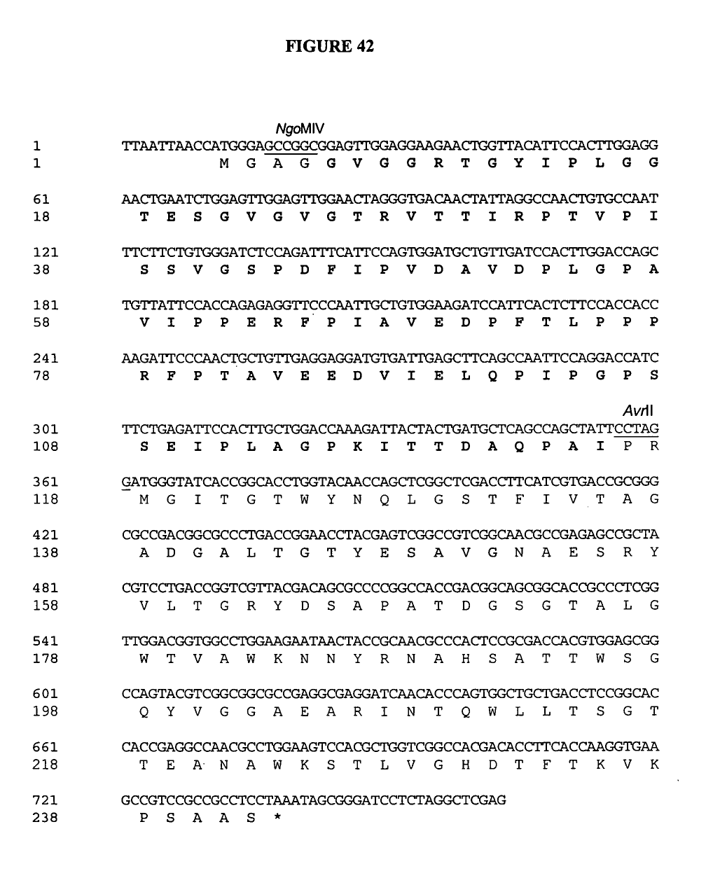

FIG. 42 shows the COPV L2-SA nucleotide and deduced amino acid sequence of the recombinant L2:SA fusion protein (242 a.a.; 25 kDa). The amino acid sequence derived from COPV L2 domain is shown in bold typeface. Underlined nucleotides indicate the NgomIV and AvrII cloning sites.

The foregoing descriptions of the invention has been presented for purposes of illustration and description. They are not intended to be exhaustive or to limit the invention to the precise form disclosed, and obviously many modifications and variations are possible in light of the above teachings. Such modifications and variations that are apparent to a person skilled in the art are intended to be included within the scope of this invention as defined by the accompanying claims.

What is claimed is:

1. A display scaffold, comprising:
An assembled virus particle comprising a) coat proteins displaying b) streptavidin.

2. A display scaffold according to claim 1 further comprising a biotinylated peptide bound to the streptavidin.

3. A display scaffold according to claim 2 wherein the biotinylated peptide is an antigen capable of eliciting an immune reaction in an animal.

4. A vaccine comprising the display scaffold according to claim 2.

5. An oral vaccine comprising the display scaffold according to claim 2.

6. A virus or virus-like particle displaying a foreign peptide sequence as a genetic fusion of to the capsid coat protein together with a mitigating peptide sequence or sequences, also present as a genetic fusion or fusions to the coat protein, such that the mitigating peptide sequence(s) improve one or more of the following characteristics of the virus or virus-like particle; a) accumulation in the host employed for production; b) yield obtained with purification; c) solubility; d) conformation of the foreign peptide sequence; and accessibility of the foreign peptide sequence.

7. A virus or virus-like particle as set forth in claim 6, where the foreign peptide sequence consists of 1 to approximately 50 amino acids.
a. A virus or virus-like particle as set forth in claim 6, where each mitigating peptide sequence consists of 1 to approximately 10 amino acids.

8. A virus or virus-like particle as set forth in claim 6, where the mitigating sequence(s) can be located at one or more of the following locations relative to the foreign peptide sequence; a) directly upstream; b) immediately downstream; c) or separated from the foreign sequence based on location within the coat protein sequence; d) or some combination of the above

9. A virus or virus-like particle as set forth in claim 6, where the foreign peptide sequence is located at or near the N-terminus of the coat protein sequence and the mitigating sequence(s) are located at or near the C-terminus of the coat protein sequence and/or in a surface exposed region of the coat protein amino acid sequence

10. A virus or virus-like particle as set forth in claim 6, where the foreign peptide sequence is located at or near the C-terminus of the coat protein sequence and the mitigating sequence(s) are located at or near the N-terminus of the coat protein sequence and/or in a surface exposed region of the coat protein amino acid sequence.

11. A virus or virus-like particle as set forth in claim 6, where the foreign peptide sequence is located within a surface exposed region of the coat protein amino acid sequence and the mitigating sequence(s) is located at or near the C-terminus of the coat protein sequence and/or at or near the N-terminus of the coat protein sequence and/or within a surface exposed region of the coat protein amino acid sequence other than the one occupied by the foreign peptide sequence.

12. A virus or virus-like particle as set forth in claim 6, derived from a population of virus or virus-like particles where the mitigating sequence or sequences consist of a randomly generated library of amino acids and selection was based on one of the properties listed in claim 6.

13. A virus or virus-like particle as set forth in claim 6, where the foreign peptide sequence consists of a single amino acid, either lysine or cysteine and the randomly generated mitigating sequence is three amino acids in length.

14. A composition as outlined in claim 6, where the virus is the tobacco mosaic virus.

15. A biotinylated virus or virus-like particle, where a virus or virus-like particle, as described in claim 6 is combined with a biotin analog capable of conjugating to the lysine or cysteine of the foreign peptide sequence, such that the biotin is covalently attached to the virus coat protein

16. 13 A composition as outlined in claim 14, where the virus is the tobacco mosaic virus.

17. A composition as outlined in claim 14, wherein the biotin analog is NHS-PEO4-biotin.

18. A composition as outlined in claim 14, wherein the biotin analog is NHS-PEO4-biotin.


SEQUENCE LISTING (84 sequences)

SEQ ID NO: 1 - protein, 256 aa, ARTIFICIAL
  GFP-AviTag fusion protein sequence
MASKGEELFTGVVPILVELDGDVNGHKFSVSGEGEGDATYGKLTLKFICTTGKLPVPWPTLVTTFSYGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITHGMDELYKMGLNDIFEAQKIEWHEG

SEQ ID NO: 2 - protein, 363 aa, ARTIFICIAL
  GFP-SA is a AA sequence of a fusion protein betweeen green fluorescent portein and Streptavidin core sequence
MASKGEELFTGVVPILVELDGDVNGHKFSVSGEGEGDATYGKLTLKFICTTGKLPVPWPTLVTTFSYGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMGLLEFVTAAGITHGMDELYKGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAAS

SEQ ID NO: 3 - DNA, 1089 bp, ARTIFICIAL
  Nucleotide sequence of GFP-SA fusion gene encoding Seq.ID. No. 2
atggctagcaaaggagaagaacttttcactggagttgtcccaattcttgttgaattagatggtgatgttaatgggcacaaattttctgtcagtggagagggtgaaggtgatgctacatacggaaagcttacccttaaatttatttgcactactggaaaactacctgttccatggccaacacttgtcactactttctcttatggtgttcaatgcttttcccgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactataactcacacaatgtatacatcacggcagacaaacaaaagaatggaatcaaagctaacttcaaaattcgccacaacattgaagatggatccgttcaactagcagaccattatcaacaaaatactccaattggcgatggccctgtccttttaccagacaaccattacctgtcgacacaatctgccctttcgaaagatcccaacgaaaagcgtgaccacatgggccttcttgagtttgtaactgctgctgggattacacatggcatggatgagctctacaaaggtatcaccggcacctggtacaaccagctcggctcgaccttcatcgtgaccgcgggcgccgacggcgccctgaccggaacctacgagtcggccgtcggcaacgccgagagccgctacgtcctgaccggtcgttacgacagcgccccggccaccgacggcagcggcaccgccctcggttggacggtggcctggaagaataactaccgcaacgcccactccgcgaccacgtggagcggccagtacgtcggcggcgccgaggcgaggatcaacacccagtggctgctgacctccggcacaaccgaggccaacgcctggaagtccacgctggtcggccacgacaccttcaccaaggtgaagccgtccgccgcctcc

SEQ ID NO: 4 - DNA, 489 bp, ARTIFICIAL
  Nucleotide sequence of Coat Protein fusion library in vector 1295. Inserted sequences are in bold and underlined.
atggnnnnknnkaaatcttacagtatcactactccatctcagttcgtgttcttgtcatcagcgtgggccgacccaatagagttaattaatttatgtactaatgccttaggaaatcagtttcaaacacaacaagctcgaactgtcgttcaaagacaattcagtgaggtgtggaaaccttcaccacaagtaactgttaggttccctgacagtgactttaaggtgtacaggtacaatgcggtattagacccgctagtcacagcactgttaggtgcattcgacactagaaatagaataatagaagttgaaaatcaggcgaaccccacgactgccgagacgttagatgctactcgtagagtagacgacgcaacggtggccataaggagcgcgataaataatttaatagtagaattgatcagaggaaccggatcttataatcggagctctttcgagagctcttctggtttggtttggacctctggtcctgcaact

SEQ ID NO: 5 - DNA, 490 bp, ARTIFICIAL
  FIGURE 12 (inserted) nucleotides are in bold. About 3600 colonies (of pLSB 1296) were resuspended off of agar plates and pooled.  DNA was purified from the E. coli, linearized with KpnI and transcribed using Ambions′ T7 mMessage mMachine transcription
catggcttcttacagtatcactactccatctcagttcgtgttcttgtcatcagcgtgggccgacccaatagagttaattaatttatgtactaatgccttaggaaatcagtttcaaacacaacaagctcgaactgtcgttcaaagacaattcagtgaggtgtggaaaccttcaccacaagtaactgttaggttccctgacagtgactttaaggtgtacaggtacaatgcggtattagacccgctagtcacagcactgttaggtgcattcgacactagaaatagaataatagaagttgaaaatcaggcgaaccccacgactgccgagacgttagatgctactcgtagagtagacgacgcaacggtggccataaggagcgcgataaataatttaatagtagaattgatcagaggaaccggatcttataatcggagctctttcgagagctcttctggtttggtttggacctctnnkaaannkggtcctgcaact

SEQ ID NO: 6 - DNA, 489 bp, ARTIFICIAL
  FIGURE 14 Nucleotide sequence of CP fusion in vector 2900. Inserted nucleotide sequences are in boldface type.
atggnnnnknnktgttcttacagtatcactactccatctcagttcgtgttcttgtcatcagcgtgggccgacccaatagagttaattaatttatgtactaatgccttaggaaatcagtttcaaacacaacaagctcgaactgtcgttcaaagacaattcagtgaggtgtggaaaccttcaccacaagtaactgttaggttccctgacagtgactttaaggtgtacaggtacaatgcggtattagacccgctagtcacagcactgttaggtgcattcgacactagaaatagaataatagaagttgaaaatcaggcgaaccccacgactgccgagacgttagatgctactcgtagagtagacgacgcaacggtggccataaggagcgcgataaataatttaatagtagaattgatcagaggaaccggatcttataatcggagctctttcgagagctcttctggtttggtttggacctctggtcctgcaact

SEQ ID NO: 7 - DNA, 489 bp, ARTIFICIAL
  FIGURE 16 Nucleotide sequence of CP fusion in vector 2901. Inserted nucleotides are in boldface.
atggcttcttacagtatcactactccatctcagttcgtgttcttgtcatcagcgtgggccgacccaatagagttaattaatttatgtactaatgccttaggaaatcagtttcaaacacaacaagctcgaactgtcgttcaaagacaattcagtgaggtgtggaaaccttcaccacaagtaactgttaggttccctgacagtgactttaaggtgtacaggtacaatgcggtattagacccgctagtcacagcactgttaggtgcattcgacactagaaatagaataatagaagttgaaaatcaggcgaaccccacgactgccgagacgttagatgctactcgtagagtagacgacgcaacggtggccataaggagcgcgataaataatttaatagtagaattgatcagaggaaccggatcttataatcggagctctttcgagagctcttctggtttggtttggacctctnnktgtnnkggtcctgcaact

SEQ ID NO: 8 - DNA, 603 bp, ARTIFICIAL
  FIGURE 18  Nucleotide sequence of CP fusion in vector 2902. Inserted sequence is in bold typeface.
atggcttcttacagtatcactactccatctcagttcgtgttcttgtcatcagcgtgggccgacccaatagagttaattaatttatgtactaatgccttaggaaatcagtttcaaacacaacaagctcgaactgtcgttcaaagacaattcagtgaggtgtggaaaccttcaccacaggtaactvnnvnnvnntgtactagacctaattacaataagagaaaaagaattcatatcggtcctggtagagctttttatactactaagaatattattggtactattagacaagctcattgtvnnvnnvnngttaggttccctgacagtgactttaaggtgtacaggtacaatgcggtattagacccgctagtcacagcactgttaggtgcattcgacactagaaatagaataatagaagttgaaaatcaggcgaaccccacgactgccgagacgttagatgctactcgtagagtagacgacgcaacggtggccataaggagcgcgataaataatttaatagtagaattgatcagaggaaccggatcttataatcggagctctttcgagagctcttctggtttggtttggacctctggtcctgcaact

SEQ ID NO: 9 - DNA, 603 bp, ARTIFICIAL
  FIGURE 20  Nucleotide sequence of CP fusion in vector 2903. Inserted sequence is in bold typeface.
atggcttcttacagtatcactactccatctcagttcgtgttcttgtcatcagcgtgggccgacccaatagagttaattaatttatgtactaatgccttaggaaatcagtttcaaacacaacaagctcgaactgtcgttcaaagacaattcagtgaggtgtggaaaccttcaccacaagtaactgttaggttccctgacagtgactttaaggtgtacaggtacaatgcggtattagacccgctagtcacagcactgttaggtgcattcgacactagaaatagaataatagaagttgaaaatcaggcgaaccccacgactgccgagacgttagatgctactcgtagagtagacgacgcaacggtggccataaggagcgcgataaataatttaatagtagaattgatcagaggaaccggatcttataatcggagctctttcgagagctcttctggtttggtttggacctctvnnvnnvnntgtactagacctaattacaataagagaaaaagaattcatatcggtcctggtagagctttttatactactaagaatattattggtactattagacaagctcattgtvnnvnnvnnggtcctgcaact

SEQ ID NO: 10 - DNA, 552 bp, ARTIFICIAL
  FIGURE 22   Nucleotide sequence of CP fusion in vector 2904. Inserted (non-TMV CP sequence) is in boldface.
atggcttcttacagtatcactactccatctcagttcgtgttcttgtcatcagcgtgggccgacccaatagagttaattaatttatgtactaatgccttaggaaatcagtttcaaacacaacaagctcgaactgtcgttcaaagacaattcagtgaggtgtggaaaccttcaccacaggtaactvnnvnnvnnttacaagcaagagtattagcagtagaaggtcaagctagagttttggctttagagvnnvnnvnngttaggttccctgacagtgactttaaggtgtacaggtacaatgcggtattagacccgctagtcacagcactgttaggtgcattcgacactagaaatagaataatagaagttgaaaatcaggcgaaccccacgactgccgagacgttagatgctactcgtagagtagacgacgcaacggtggccataaggagcgcgataaataatttaatagtagaattgatcagaggaaccggatcttataatcggagctctttcgagagctcttctggtttggtttggacctctggtcctgcaact

SEQ ID NO: 11 - DNA, 521 bp, ARTIFICIAL
  FIGURE 24 Nucleotide sequence of CP fusion in vector 2905. Non-TMV sequence is in bold.
atggnnnnknnkaattggtttgatatcactgccvnnvnnvnnvnnvnntcttacagtatcactactccatctcagttcgtgttcttgtcatcagcgtgggccgacccaatagagttaattaatttatgtactaatgccttaggaaatcagtttcaaacacaacaagctcgaactgtcgttcaaagacaattcagtgaggtgtggaaaccttcaccacaagtaactgttaggttccctgacagtgactttaaggtgtacaggtacaatgcggtattagacccgctagtcacagcactgttaggtgcattcgacactagaaatagaataatagaagttgaaaatcaggcgaaccccacgactgccgagacgttagatgctactcgtagagtagacgacgcaacggtggccataaggagcgcgataaataatttaatagtagaattgatcagaggaaccggatcttataatcggagctctttcgagagctcttctggtttggtttggacctctggtcctgcaac

SEQ ID NO: 12 - DNA, 540 bp, ARTIFICIAL
  FIGURE 26  Nucleotide sequence of CP fusion in vector 2907. Non-TMV sequences are in bold
atggnnnnknnkttgtggaattggtttgatatcactaactggttatgggccvnnvnnvnnvnnvnntcttacagtatcactactccatctcagttcgtgttcttgtcatcagcgtgggccgacccaatagagttaattaatttatgtactaatgccttaggaaatcagtttcaaacacaacaagctcgaactgtcgttcaaagacaattcagtgaggtgtggaaaccttcaccacaagtaactgttaggttccctgacagtgactttaaggtgtacaggtacaatgcggtattagacccgctagtcacagcactgttaggtgcattcgacactagaaatagaataatagaagttgaaaatcaggcgaaccccacgactgccgagacgttagatgctactcgtagagtagacgacgcaacggtggccataaggagcgcgataaataatttaatagtagaattgatcagaggaaccggatcttataatcggagctctttcgagagctcttctggtttggtttggacctctggtcctgcaact

SEQ ID NO: 13 - DNA, 555 bp, ARTIFICIAL
  FIGURE 28  Nucleotide sequence of CP fusion in vector 2908. Inserted nucleotide sequences are in bold.
atggnnnnknnkaatgagcaagagttgttagaattggataaatgggcttctttgtggaatgcctctvnnvnnvnnvnnvnntcttacagtatcactactccatctcagttcgtgttcttgtcatcagcgtgggccgacccaatagagttaattaatttatgtactaatgccttaggaaatcagtttcaaacacaacaagctcgaactgtcgttcaaagacaattcagtgaggtgtggaaaccttcaccacaagtaactgttaggttccctgacagtgactttaaggtgtacaggtacaatgcggtattagacccgctagtcacagcactgttaggtgcattcgacactagaaatagaataatagaagttgaaaatcaggcgaaccccacgactgccgagacgttagatgctactcgtagagtagacgacgcaacggtggccataaggagcgcgataaataatttaatagtagaattgatcagaggaaccggatcttataatcggagctctttcgagagctcttctggtttggtttggacctctggtcctgcaact

SEQ ID NO: 14 - protein, 513 aa, COPV
  COPV L2 protein
MALIRKRRAAPQDIYPACKVSNTCPADILNKMEQNTLADKILKYGSAGVFLGGLGISTGKGVGGRTGYIPLGGTESGVGVGTRVTTIRPTVPISSVGSPDFIPVDAVDPLGPAVIPPERFPIAVEDPFTLPPPRFPTAVEEDVIELQPIPGPSSEIPLAGPKITTDAQPAILEVIPETRPPKVITRHQYSNPAFEVSITSNSGAGESSASDHVLVEGFSGGHSIGEHIPLQDLAPSRPSFSETIEDETAFSSSTPKQGSRSERPKSYYNRRRYQQVQVTDPVFISRPRSLVTFDNPAFDESVDLIFERDVAEITAAPHADFTDITKLTKPAYHRGPSGHVRVSRLGHRANIKTRSGLTIGPQSHFYYDVSSIDPAESFELQALGNVSSAEQTGEAVISSGTGDFEIISLEDSILESYNDEDLIDVFEDVARDLHLLVGERRQQPIQVQRYIKPFSFVNEGVHIIHPGSESDFWLPPVTPDSTPAIVIDILDSSADYYLHPSLIKKRKRKHFFF

SEQ ID NO: 15 - DNA, 648 bp, artificial
  COPV C1 7-2 Light Chain
atggatatccagatgacacagactacctcctccctgtctgcctctctgggcgacagagtcaccatcagttgctgggcaagtcaggacattaccagttatttaaactggtatcaacagaaaccagatggaactgttaaactcctaatctacttcacatcaagattacattcaggagtcccattgagattcagtggcagtgggtctggaacagagtattctctcaccattagcaacctggagcaagaagatcttgccacttacttttgccaacagggtaagacgcttcctcggacgttcggtggaggcaccaaactggagatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 16 - DNA, 1341 bp, ARTIFICIAL
  COPV C1 7-2 Heavy Chain
atggaggtccagcttcagcagtcaggacctggggtggtgaaacctggggcctcagtgaggatatcctgcaagacttctggatacacagtctctgcctacaatatacactggatgaaacagagccatggaaagagccttgaatggattggatatatttctccttataatggtgatactgactacaaccagaaattcaagaacaaggccacattgactgtagacatttcctccaacacagcctacatggaactccgcagcctgacatctgaggactctgccgtctattactgtgcgcgctacggtagtggaaggggagactatgctatggactattggggtcaaggaacctcagtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 17 - DNA, 1431 bp, ARTIFICIAL
  COPV C1 7-2 FAB
atggatatccagatgacacagactacctcctccctgtctgcctctctgggcgacagagtcaccatcagttgctgggcaagtcaggacattaccagttatttaaactggtatcaacagaaaccagatggaactgttaaactcctaatctacttcacatcaagattacattcaggagtcccattgagattcagtggcagtgggtctggaacagagtattctctcaccattagcaacctggagcaagaagatcttgccacttacttttgccaacagggtaagacgcttcctcggacgttcggtggaggcaccaaactggagatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgtggaggtaaacgtacgatacaggattctgcaactgatacagttgacttaggtgcagagttgcatagagatgaccctccacctactgcttctgatatcggaaagcgaggcggagaggtccagcttcagcagtcaggacctggggtggtgaaacctggggcctcagtgaggatatcctgcaagacttctggatacacagtctctgcctacaatatacactggatgaaacagagccatggaaagagccttgaatggattggatatatttctccttataatggtgatactgactacaaccagaaattcaagaacaaggccacattgactgtagacatttcctccaacacagcctacatggaactccgcagcctgacatctgaggactctgccgtctattactgtgcgcgctacggtagtggaaggggagactatgctatggactattggggtcaaggaacctcagtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttga

SEQ ID NO: 18 - DNA, 2094 bp, ARTIFICIAL
  COPV 1 7-2 mAb
atggatatccagatgacacagactacctcctccctgtctgcctctctgggcgacagagtcaccatcagttgctgggcaagtcaggacattaccagttatttaaactggtatcaacagaaaccagatggaactgttaaactcctaatctacttcacatcaagattacattcaggagtcccattgagattcagtggcagtgggtctggaacagagtattctctcaccattagcaacctggagcaagaagatcttgccacttacttttgccaacagggtaagacgcttcctcggacgttcggtggaggcaccaaactggagatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcactctcacgttgaccaaggacgagtatgaacgacataacagctatacctgtgaggccactcacaagacatcaacttcacccattgtcaagagcttcaacaggaatgagtgtggaggtaaacgtacgatacaggattctgcaactgatacagttgacttaggtgcagagttgcatagagatgaccctccacctactgcttctgatattggaaagcgaggcggagaggtccagcttcagcagtcaggacctggggtggtgaaacctggggcctcagtgaggatatcctgcaagacttctggatacacagtctctgcctacaatatacactggatgaaacagagccatggaaagagccttgaatggattggatatatttctccttataatggtgatactgactacaaccagaaattcaagaacaaggccacattgactgtagacatttcctccaacacagcctacatggaactccgcagcctgacatctgaggactctgccgtctattactgtgcgcgctacggtagtggaaggggagactatgctatggactattggggtcaaggaacctcagtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 19 - DNA, 1440 bp, ARTIFICIAL
  HPV-16 G4 FAB
atggacattgtgctgacccaatctccagcttctttgactgtgtctctagggcagagggccaccatctcctgcaaggccagccaaagtgttgattatgatggtgatagttatatgaactggtaccagcagaaaccaggacagccacccaaactcctcatctatgctgcatccaatctagaatctgggatcccagccaggtttagtggcagtgggtctgggacagacttcaccctcaacatccatcctgtggaggaggaggatgctgcaacctattactgtcagcaaggtaatgaggatccattcacgttcggctcggggacaaagttggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgtggaggtaaacgtacgatacaggattctgcaactgatacagttgacttaggtgcagagttgcatagagatgaccctccacctactgcttctgatatcggaaagcgaggcggagaggttcagctccagcagtctgggactgtgctggcaaggcctggggcttccgtgaagatgtcctgcaaggcttctggctacagctttaccagctactggatgcactgggtaaagcagaggcctggacagggtctagaatggattggtgctatttatcctggaaatagtgatactaactacaaccagaagttcaagggcaaggccaaactgactgcagtcacatccgccaccactgcctacatggaattcagcagcctgacaaatgaggactctgcggtctattactgttcaagaaatggtaactttgactcttggggccaaggcaccactctcacagtctcctcagccaaaacaacagccccatcagtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtgactag

SEQ ID NO: 20 - DNA, 2106 bp, ARTIFICIAL
  HPV-16 G4 mAb
atggacattgtgctgacccaatctccagcttctttgactgtgtctctagggcagagggccaccatctcctgcaaggccagccaaagtgttgattatgatggtgatagttatatgaactggtaccaacagaaaccaggacagccacccaaactcctcatctatgctgcatccaatctagaatctgggatcccagccaggtttagtggcagtgggtctgggacagacttcaccctcaacatccatcctgtggaggaggaggatgctgcaacctattactgtcagcaaggtaatgaggatccattcacgttcggctcggggacaaagttggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgtggaggtaaacgtacgatacaggattctgcaactgatacagttgacttaggtgcagagttgcatagagatgaccctccacctactgcttctgatatcggaaagcgaggcggagaggttcagctccagcagtctgggactgtgctggcaaggcctggggcttccgtgaagatgtcctgcaaggcttctggctacagctttaccagctactggatgcactgggtaaagcagaggcctggacagggtctagaatggattggtgctatttatcctggaaatagtgatactaactacaaccagaagttcaagggcaaggccaaactgactgcagtcacatccgccaccactgcctacatggaattcagcagcctgacaaatgaggactctgcggtctattactgttcaagaaatggtaactttgactcttggggccaaggcaccactctcacagtctcctcagccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 21 - protein, 242 aa, Artificial
  Deduced amino acid sequence of COPV L2-SA fusion protein (from SEQ ID NO 84)
MGAGGVGGRTGYIPLGGTESGVGVGTRVTTIRPTVPISSVGSPDFIPVDAVDPLGPAVIPPERFPIAVEDPFTLPPPRFPTAVEEDVIELQPIPGPSSEIPLAGPKITTDAQPAIPRMGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAAS

SEQ ID NO: 22 - protein, 16 aa, Artificial
  This 16 aa tag is referred to as the “aviTag” sequence (GLNDIFEAQKIEWHEG).   Proteins with this tag, at either the N or C terminus, can be biotinylated by birA.
GLNDIFEAQKIEWHEG

SEQ ID NO: 23 - DNA, 46 bp, Artificial
  JAL 604 Forward oligo
ttgttaattaaccatgggaaaggataacaccgtgccactgaaattg

SEQ ID NO: 24 - DNA, 40 bp, Artificial
  JAL 605  Reverse oligo
ctttctagattatttttctgcactacgcagggatatttca

SEQ ID NO: 25 - DNA, 42 bp, Artificial
  JAL 606 forward oligo
gggtctagagaaggattaattaaatggctagcaaaggagaag

SEQ ID NO: 26 - DNA, 50 bp, Artificial
  JAL 607 Reverse oligo
gtgcctcgaatatatcatttaaaccagatttgtagagctcatccatgcca

SEQ ID NO: 27 - DNA, 59 bp, Artificial
  JAL 608 reverse primer
ccccctaggttaaccctcatgccactctattttttgtgcctcgaatatatcatttaaac

SEQ ID NO: 28 - protein, 163 aa, Artificial
  pLSB 1295.4 N-Terminal addition M ADFK SYS
MADFKSYSITTPSQFVFLSSAWADPIELINLCTNALGNQFQTQQARTVVQRQFSEVWKPSPQVTVRFPDSDFKVYRYNAVLDPLVTALLGAFDTRNRIIEVENQANPTTAETLDATRRVDDATVAIRSAINNLIVELIRGTGSYNRSSFESSSGLVWTSGPAT

SEQ ID NO: 29 - DNA, 44 bp, Artificial
  JAL 609 oligo
atgagctctacaaaggtatcaccggcacctggtacaaccagctc

SEQ ID NO: 30 - DNA, 38 bp, Artificial
  JAL 610 oligo
aaacctaggttaggaggcggcggacggcttcaccttgg

SEQ ID NO: 31 - DNA, 47 bp, Artificial
  JAL 613 oligo
cgaaccatggnnnnknnkaaatcttacagtatcactactccatctca

SEQ ID NO: 32 - DNA, 29 bp, Artificial
  JAL 590 oligo
gccaacacatccgggtacctgggccccta

SEQ ID NO: 33 - DNA, 34 bp, Artificial
  JAL 619 oligo
gccttggtacctgggcccctaccgggggtaacgg

SEQ ID NO: 34 - DNA, 23 bp, Artificial
  JAL 618 oligo
cgatgatgattcggaggctactg

SEQ ID NO: 35 - DNA, 19 bp, Artificial
  JAL 153 oligo
aggctactgtcgccgaatc

SEQ ID NO: 36 - protein, 159 aa, Tobacco Mosaic Virus U1
MSYSITTPSQFVFLSSAWADPIELINLCTNALGNQFQTQQARTVVQRQFSEVWKPSPQVTVRFPDSDFKVYRYNAVLDPLVTALLGAFDTRNRIIEVENQANPTTAETLDATRRVDDATVAIRSAINNLIVELIRGTGSYNRSSFESSSGLVWTSGPAT

SEQ ID NO: 37 - protein, 9 aa, Artificial
  isolate showing a mutated coat protein of TMV
LVWTSSNAT

SEQ ID NO: 38 - protein, 12 aa, Artificial
  isolate of mutated TMV coat protein
LVWTSNKEGPAT

SEQ ID NO: 39 - protein, 7 aa, Artificial
  isolate of mutated TMV coat protein
LVWTSAT

SEQ ID NO: 40 - DNA, 44 bp, ARTIFICIAL
  JAL 634 oligo
cgaccatggnnnnknnktgttcttacagtatcactactccatct

SEQ ID NO: 41 - DNA, 24 bp, ARTIFICIAL
  JAL 70 forward oligo
cgtccatggcttcttacagtatca

SEQ ID NO: 42 - DNA, 40 bp, ARTIFICIAL
  JAL 635 oligo
ggaccmnnacamnnagaggtccaaaccaaaccagaagagc

SEQ ID NO: 43 - DNA, 31 bp, ARTIFICIAL
  JAL 614 oligo
cctgcaacttgaggtagtcaagatgcataat

SEQ ID NO: 44 - protein, 13 aa, HIV Virus
LWNWFDITNWLWA

SEQ ID NO: 45 - protein, 16 aa, HIV Virus
NEQELLELDKWASLWN

SEQ ID NO: 46 - protein, 9 aa, ARTIFICIAL
  Coat Protein AA isolate of p1296 series
LVWTSGPAT

SEQ ID NO: 47 - protein, 7 aa, Artificial
  GKG sequence was placed at the N- Terminus of the TMV Coat Protein sequence
MGKGSYS

SEQ ID NO: 48 - protein, 8 aa, Artificial
  pLSB 1295.1 N-Terminal addition  M AEFK SYS
MAEFKSYS

SEQ ID NO: 49 - protein, 8 aa, Artificial
  pLSB 1295.2 N-Terminal addition  M EVLK SYS
MEVLKSYS

SEQ ID NO: 50 - protein, 8 aa, Artificial
  pLSB 1295.3 N-Terminal addition  M AEVK SYS
MAEVKSYS

SEQ ID NO: 51 - protein, 8 aa, Artificial
  pLSB 1295.4 N-Terminal addition M ADFK SYS
MADFKSYS

SEQ ID NO: 52 - protein, 8 aa, ARTIFICIAL
  pLSB 1295.6 N-Terminal addition  M DVEK SYS
MDVEKSYS

SEQ ID NO: 53 - protein, 8 aa, Artificial
  pLSB 1295.9 N-Terminal addition  M EGAK SYS
MEGAKSYS

SEQ ID NO: 54 - protein, 8 aa, Artificial
  pLSB 1295.10 N-Terminal addition   M EPMK SYS
MEPMKSYS

SEQ ID NO: 55 - protein, 8 aa, Artificial
  pLSB 1295.11 N-Terminal addition M EMGK SYS
MEMGKSYS

SEQ ID NO: 56 - protein, 8 aa, Artificial
  pLSB 1295.12 N-Terminal addition M DGAK SYS
MDGAKSYS

SEQ ID NO: 57 - protein, 8 aa, Artificial
  pLSB 1295.13 N-Terminal addition  M DGAK SYS
MEGLKSYS

SEQ ID NO: 58 - protein, 8 aa, Artificial
  pLSB 1295.14 N-Terminal addition  M VEFK SYS
MVEFKSYS

SEQ ID NO: 59 - protein, 9 aa, Tobacco Mosaic Virus U1
LVWTSGPAT

SEQ ID NO: 60 - protein, 159 aa, Artificial
  pLSB 1296.1  LVWTSSNAT  SN is substituted for GP in LVWTSGPAT
MSYSITTPSQFVFLSSAWADPIELINLCTNALGNQFQTQQARTVVQRQFSEVWKPSPQVTVRFPDSDFKVYRYNAVLDPLVTALLGAFDTRNRIIEVENQANPTTAETLDATRRVDDATVAIRSAINNLIVELIRGTGSYNRSSFESSSGLVWTSSNAT

SEQ ID NO: 61 - protein, 162 aa, Artificial
  1296.5 LVWTSNKEGPAT where NKE is inserted into the c-terminal sequence of TMV coat protein
MSYSITTPSQFVFLSSAWADPIELINLCTNALGNQFQTQQARTVVQRQFSEVWKPSPQVTVRFPDSDFKVYRYNAVLDPLVTALLGAFDTRNRIIEVENQANPTTAETLDATRRVDDATVAIRSAINNLIVELIRGTGSYNRSSFESSSGLVWTSNKEGPAT

SEQ ID NO: 62 - protein, 157 aa, Artificial
  pLSB 1296.7 LVWTSAT where GP is deleted from the C-Terminus of TMV coat protein sequence LVWTSGPAT
MSYSITTPSQFVFLSSAWADPIELINLCTNALGNQFQTQQARTVVQRQFSEVWKPSPQVTVRFPDSDFKVYRYNAVLDPLVTALLGAFDTRNRIIEVENQANPTTAETLDATRRVDDATVAIRSAINNLIVELIRGTGSYNRSSFESSSGLVWTSAT

SEQ ID NO: 63 - protein, 12 aa, Artificial
  TOBAMOVIRUS Coat Protein AA isolate of p2900 series
MDQGCSYSITTP

SEQ ID NO: 64 - protein, 13 aa, Artificial
  TOBAMOVIRUS Coat Protein AA isolate of p2900 series
MVGACSYSYITTP

SEQ ID NO: 65 - protein, 13 aa, Artificial
  TOBAMOVIRUS Coat Protein AA isolate of p2900 series
MVVGCSYSYITTP

SEQ ID NO: 66 - protein, 13 aa, Artificial
  TOBAMOVIRUS Coat Protein AA isolate of p2900 series
MVEGCSYSYITTP

SEQ ID NO: 67 - protein, 13 aa, Artificial
  TOBAMOVIRUS Coat Protein AA isolate of p2900 series
MVAMCSYSYITTP

SEQ ID NO: 68 - protein, 13 aa, Artificial
  TOBAMOVIRUS Coat Protein AA isolate of p2900 series
MAGGCSYSYITTP

SEQ ID NO: 69 - protein, 13 aa, Artificial
  TOBAMOVIRUS Coat Protein AA isolate of p2900 series
MASTCSYSYITTP

SEQ ID NO: 70 - protein, 13 aa, Artificial
  TOBAMOVIRUS Coat Protein AA isolate of p2900 series
MERTCSYSYITTP

SEQ ID NO: 71 - protein, 163 aa, ARTIFICIAL
  MASYSITTP.....WTSACLGPAT  Amino Acid Addition at each terminal A at N-terminus and ACL at C terminus
MASYSITTPSQFVFLSSAWADPIELINLCTNALGNQFQTQQARTVVQRQFSEVWKPSPQVTVRFPDSDFKVYRYNAVLDPLVTALLGAFDTRNRIIEVENQANPTTAETLDATRRVDDATVAIRSAINNLIVELIRGTGSYNRSSFESSSGLVWTSACLGPAT

SEQ ID NO: 72 - protein, 163 aa, Artificial
  pLSB 2901.3   MASYSITTP.....WTS DCC GPAT Addition of A at the 2 position and addition of DCC prior to GPAT at the C-terminus
MASYSITTPSQFVFLSSAWADPIELINLCTNALGNQFQTQQARTVVQRQFSEVWKPSPQVTVRFPDSDFKVYRYNAVLDPLVTALLGAFDTRNRIIEVENQANPTTAETLDATRRVDDATVAIRSAINNLIVELIRGTGSYNRSSFESSSGLVWTSDCCGPAT

SEQ ID NO: 73 - protein, 163 aa, artificial
  pLSB 2901.4 MASYSITTP.....WTS LCP GPAT Addition of A at the 2 positioin and addition of LPC prior to GPAT at the C-terminus
MASYSITTPSQFVFLSSAWADPIELINLCTNALGNQFQTQQARTVVQRQFSEVWKPSPQVTVRFPDSDFKVYRYNAVLDPLVTALLGAFDTRNRIIEVENQANPTTAETLDATRRVDDATVAIRSAINNLIVELIRGTGSYNRSSFESSSGLVWTSLCPGPAT

SEQ ID NO: 74 - protein, 163 aa, Artificial
  pLSB 2901.6 MASYSITTP.....WTS  PCP GPAT Addition of A at the 2 position and addition of PCP prior to GPAT at the C-terminus
MASYSITTPSQFVFLSSAWADPIELINLCTNALGNQFQTQQARTVVQRQFSEVWKPSPQVTVRFPDSDFKVYRYNAVLDPLVTALLGAFDTRNRIIEVENQANPTTAETLDATRRVDDATVAIRSAINNLIVELIRGTGSYNRSSFESSSGLVWTSPCPGPAT

SEQ ID NO: 75 - protein, 163 aa, Artificial
  pLSB 2901.7 MASYSITTP.....WTS TCA GPAT Addition of A at the 2 position and addition of TCA prior to GPAT at the C-Terminus
MASYSITTPSQFVFLSSAWADPIELINLCTNALGNQFQTQQARTVVQRQFSEVWKPSPQVTVRFPDSDFKVYRYNAVLDPLVTALLGAFDTRNRIIEVENQANPTTAETLDATRRVDDATVAIRSAINNLIVELIRGTGSYNRSSFESSSGLVWTSTCAGPAT

SEQ ID NO: 76 - protein, 163 aa, Artificial
  pLSB 2901.13 MASYSITTP.....WTS QCP GPAT Addition of A at the 2 position and addition of QCP Prior to GPAT at the C-Terminus
MASYSITTPSQFVFLSSAWADPIELINLCTNALGNQFQTQQARTVVQRQFSEVWKPSPQVTVRFPDSDFKVYRYNAVLDPLVTALLGAFDTRNRIIEVENQANPTTAETLDATRRVDDATVAIRSAINNLIVELIRGTGSYNRSSFESSSGLVWTSQCPGPAT

SEQ ID NO: 77 - protein, 163 aa, Artificial
  pLSB 2901.16 MASYSITTP.....WTS VCL GPAT Addition of A at the 2 position and addition of VCL Prior to GPAT at the C-Terminus
MASYSITTPSQFVFLSSAWADPIELINLCTNALGNQFQTQQARTVVQRQFSEVWKPSPQVTVRFPDSDFKVYRYNAVLDPLVTALLGAFDTRNRIIEVENQANPTTAETLDATRRVDDATVAIRSAINNLIVELIRGTGSYNRSSFESSSGLVWTSVCLGPAT

SEQ ID NO: 78 - protein, 163 aa, ARTIFICIAL
  pLSB 2901.23 MASYSITTP.....WTS SCR GPAT Addition of A at the 2 position and addition of SCR Prior to GPAT at the C-Terminus
MASYSITTPSQFVFLSSAWADPIELINLCTNALGNQFQTQQARTVVQRQFSEVWKPSPQVTVRFPDSDFKVYRYNAVLDPLVTALLGAFDTRNRIIEVENQANPTTAETLDATRRVDDATVAIRSAINNLIVELIRGTGSYNRSSFESSSGLVWTSSCRGPAT

SEQ ID NO: 79 - protein, 7 aa, ARTIFICIAL
  streptavidin-specific heptapeptide
TLIAHPE

SEQ ID NO: 80 - protein, 4 aa, TMV
  final 4 codons of the Coat Protein
GPAT

SEQ ID NO: 81 - protein, 9 aa, artificial
  H-2d restricted peptide from EGFP
HYLSTQSAL

SEQ ID NO: 82 - protein, 5 aa, artificial
  fusion sequence
GKGAG

SEQ ID NO: 83 - protein, 4 aa, artificial
  fusion sequence
ADFK

SEQ ID NO: 84 - DNA, 763 bp, artificial
  Nucleotide sequence of the recombinant COPV L2SA fusion protein
ttaattaaccatgggagccggcggagttggaggaagaactggttacattccacttggaggaactgaatctggagttggagttggaactagggtgacaactattaggccaactgtgccaatttcttctgtgggatctccagatttcattccagtggatgctgttgatccacttggaccagctgttattccaccagagaggttcccaattgctgtggaagatccattcactcttccaccaccaagattcccaactgctgttgaggaggatgtgattgagcttcagccaattccaggaccatcttctgagattccacttgctggaccaaagattactactgatgctcagccagctattcctaggatgggtatcaccggcacctggtacaaccagctcggctcgaccttcatcgtgaccgcgggcgccgacggcgccctgaccggaacctacgagtcggccgtcggcaacgccgagagccgctacgtcctgaccggtcgttacgacagcgccccggccaccgacggcagcggcaccgccctcggttggacggtggcctggaagaataactaccgcaacgcccactccgcgaccacgtggagcggccagtacgtcggcggcgccgaggcgaggatcaacacccagtggctgctgacctccggcaccaccgaggccaacgcctggaagtccacgctggtcggccacgacaccttcaccaaggtgaagccgtccgccgcctcctaaatagcgggatcctctaggctcgag